(19) United States
(12) Patent Application Publication
(10) Pub. No.: US 2025/0041384 A1
(43) Pub. Date: Feb. 6, 2025

(54) RESCUING VOLTAGE-GATED SODIUM CHANNEL FUNCTION IN INHIBITORY NEURONS

(71) Applicants: Allen Institute, Seattle, WA (US); Seattle Children's Hospital d/b/a Seattle Children's Research Institute, Seattle, WA (US)

(72) Inventors: John K. Mich, Seattle, WA (US); Edward Sebastian Lein, Mercer Island, WA (US); Jonathan Ting, Lake Forest Park, WA (US); Boaz P. Levi, Seattle, WA (US); Erik Hess, Issaquah, WA (US); Franck Kalume, Bothell, WA (US)

(73) Assignees: Allen Institute, Seattle, WA (US); Seattle Children's Hospital d/b/a Seattle Children's Research Institute, Seattle, WA (US)

(21) Appl. No.: 18/920,747

(22) Filed: Oct. 18, 2024

Related U.S. Application Data
(63) Continuation of application No. 17/044,232, filed on Sep. 30, 2020, now Pat. No. 12,121,563.
(60) Provisional application No. 62/655,043, filed on Apr. 9, 2018.
(60) Provisional application No. 62/742,835, filed on Oct. 8, 2018.
(60) Provisional application No. 62/810,281, filed on Feb. 25, 2019.

(51) Int. Cl. A61K 38/17; A61K 9/00; A61K 48/00; C07K 14/705; C12N 15/86

(52) U.S. Cl. CPC A61K 38/177; A61K 9/0019; A61K 48/0058; C07K 14/705; C12N 15/86; C12N 2750/14143; C12N 2830/008

(57) ABSTRACT

Selectively providing voltage-gated sodium channel function sufficient to rescue impaired Nav1.1 function to inhibitory neurons is described. Provided voltage-gated sodium channel function sufficient to rescue impaired Nav1.1 function in inhibitory neurons can be used to treat disorders such as epilepsy, and more particularly, Dravet Syndrome.

### CROSS-REFERENCE TO RELATED APPLICATIONS

This application is a continuation application based on U.S. patent application Ser. No. 17/044,232, filed on Sep. 30, 2020, which is a U.S. National Phase Application based on International Patent Application No. PCT/US2019/026638, filed on Apr. 9, 2019, which claims priority to U.S. Provisional Patent Application Nos. 62/655,043, filed Apr. 9, 2018, 62/742,835, filed Oct. 8, 2018, and 62/810,281, filed Feb. 25, 2019, each of which is incorporated herein by reference in its entirety as if fully set forth herein.

### STATEMENT REGARDING FEDERALLY SPONSORED RESEARCH OR DEVELOPMENT

This invention was made with government support under grant RF1MH114126 awarded by the National Institutes of Health. The government has certain rights in the invention.

### REFERENCE TO SEQUENCE LISTING

The Sequence Listing associated with this application is provided in xml format in lieu of a paper copy, and is hereby incorporated by reference into the specification. The name of the text file containing the Sequence Listing is 3BH3396_XML. The text is 647, 168 bytes, was created on Oct. 15, 2024, and is being submitted electronically via Patent Center.

### FIELD OF THE DISCLOSURE

The current disclosure describes the rescue of voltage-gated sodium channel function selectively in inhibitory neurons in need thereof. Rescued voltage-gated sodium channel function in inhibitory neurons can be used to treat disorders such as epilepsy, and more particularly, Dravet Syndrome.

### BACKGROUND OF THE DISCLOSURE

There are numerous neurological disorders for which treatments are urgently needed. One class of such disorders arise due to dysfunctional Nav1.1 sodium channels in inhibitory neurons. For example, epilepsy, infantile spasms, migraine headaches, and autism spectrum disorders are associated with dysfunctional Nav1.1 sodium channels.

Epilepsy is a neurological disorder that occurs when the brain presents an enduring predisposition to generate two or more epileptic seizures. An epileptic seizure is a temporary disruption of brain function due to abnormal excessive or synchronous neuronal activity. Its manifestation may include periods of unusual behavior, sensations and sometimes loss of consciousness.

Dravet Syndrome (DS) particularly is a rare and catastrophic form of intractable epilepsy that begins in infancy. Initially, the patient experiences prolonged seizures. In their second year, additional types of seizure begin to occur and this typically coincides with a developmental decline. This leads to poor development of language and motor skills.

Children with DS are likely to experience multiple seizures per day. Epileptic seizures are far more likely to result in death in sufferers of DS; 10 to 16% of patients diagnosed with DS die in childhood, particularly between two and four years of age. Additionally, patients are at risk of numerous associated conditions including orthopedic developmental issues, impaired growth, sleep and circadian rhythm impairments, and chronic infections.

Of particular concern, children with DS are particularly susceptible to episodes of Status epilepticus. Status epilepticus is a condition in which a seizure lasts for more than 5 minutes or multiple seizures occur close together within a 5 minute-period without recovery of consciousness between them. This severe condition is categorized as a medical emergency requiring immediate medical intervention, typically involving hospitalization. Prolonged convulsive status epilepticus lasting >30 min can be fatal and lead to substantial brain damage. Frequent hospitalizations of children with DS are clearly distressing, not only to the patient but also to family and care givers. The cost of care for DS is also high as the affected children require constant supervision and many require institutionalization.

At present, although a number of anticonvulsant therapies can be employed to reduce the instance of seizures in patients with DS, the results obtained with such therapies are typically poor and those therapies only produce partial cessation of seizures in most patients. Many of these anticonvulsants such as clobazam and clonazepam have undesirable side effects, which are particularly acute in pediatric patients. Furthermore, certain anticonvulsants (particularly the sodium-channel blockers) exacerbate the seizures.

Cell-type or cell-class specific gene delivery using non-pathogenic viral delivery is showing increasing promise for the treatment of diverse diseases. Inclusion of particular gene regulatory elements, such as specific promoters or enhancers, within the delivered vector, has been beneficial to provide specificity for gene expression within particular targeted cell types. For example, Dimidschstein and colleagues (Nat Neurosci 19(12): 1743-1749, 2016) developed a viral delivery gene construct based on the adeno-associated virus (AAV) that resulted in selective expression of a gene within gamma-aminobutyric acid (GABA) ergic interneurons within the telencephalon, a cell type important in the treatment of epilepsy. This construct included a 529 base pair (bp) enhancer sequence (referred to as ml56i or mDIx).

One significant drawback to using AAV as a selective gene-delivery system is the strictly restricted packaging limit of AAVs; this is particularly limiting to the inclusion of lengthy genetic control elements. In addition, existing interneuron-specific AAV expression constructs provide weak expression in certain applications, as well as for expression of transgenes (such as therapeutic genes) that are more poorly tolerated than other more commonly used proteins. Thus, there remains a need in the art for shorter enhancer sequences that are capable of providing rapid and strong expression of functional proteins within selected cell types.

### SUMMARY OF THE DISCLOSURE

The current disclosure provides expression constructs that result in unexpectedly rapid and high levels of protein expression selectively within inhibitory neurons for the purpose of rescuing defective Nav1.1 channel function. In particular embodiments, the current disclosure provides a concatemerized core of the human 156i enhancer and a gene encoding a voltage-gated sodium channel protein or nucleotide sequence that can rescue impaired Nav1.1 sodium channel function. The expression constructs can be used to reverse or ameliorate the effects of Nav1.1 voltage-gated sodium channel dysfunction in inhibitory neurons. In particular embodiments, the current disclosure provides treatment of sodium channel disorders by selectively delivering a gene allowing for a functioning voltage-gated sodium channel to inhibitory neurons. Administration of viral vectors including these enhancers and genes results in selective expression of a protein or nucleotide sequence that rescues Nav1.1 sodium channel function in inhibitory interneurons. In particular embodiments, the therapeutic voltage-gated sodium channel genes in these viral vectors can be of bacterial origin, which are small and can fit into a single AAV construct, or of human origin, which are larger and can use a dual construct AAV delivery technique, among other strategies. The Nav1.1 sodium channel disorders that can be treated include epilepsy, and more particularly, Dravet Syndrome (DS). For example, as disclosed herein, administration of the expression constructs results in therapeutic efficacy to treat DS in a well-established in vivo mouse model of the disease.

### BRIEF DESCRIPTION OF THE SEVERAL VIEWS OF THE DRAWINGS

Many of the drawings submitted herein are better understood in color. Applicant considers the color versions of the drawings as part of the original submission and reserve the right to present color images of the drawings in later proceedings.

FIGS. 1A-1D. Pan-GABAergic viral enhancer labels fast spiking interneurons in human organotypic slice culture. (FIG. 1A) Green fluorescence identifies cells expressing a CN1180/DJ viral vector at 7 DIV/DPI (DIV: days in vitro, DPI: days post infection). CN1180 includes a SYFP2 expression cassette under control of the human DLX (hDLX) I12b enhancer and a minimal beta-globin promoter. Neurons were patch-clamped and visualized post-hoc with Alexa 594 backfill in the red channel. (FIG. 1B) Morphologies of patched cells from one human case, categorized by presence of YFP fluorescence, suggest that SYFP2-expressing cells are largely interneurons. (FIG. 1C) Electrophysiological parameters of YFP+ neurons suggest that these cells are predominantly fast-spiking interneurons. (FIG. 1D) Post-hoc immuno-histochemistry demonstrates that many YFP+ cells (arrows) express Parvalbumin (PVALB), which suggests that these cells have molecular properties of fast-spiking interneurons.

FIG. 2 depicts an exemplary vector for targeted expression of a protein in inhibitory interneurons: CN1367-rAAV2-hl56i-minBglobin-His-NavMs-P2A-SYFP2-WPRE3-BGHpA. Key: hl56i—full-length human DLX 156i enhancer (SEQ ID NO: 1); minBglobin—minimal beta globin promoter; His-NavMs—Hexahistidine-tagged voltage-gated sodium channel from Magnetococcus marinus; P2A—self-cleaving peptide; SYFP2—super yellow fluorescent protein 2; WPRE3—woodchuck hepatitis virus posttranscriptional regulatory element 3 (SEQ ID NO: 19); BGHpA-bovine growth hormone polyA sequence.

FIG. 3. Virus CN1244/PHP.eB. 10¹¹ genome copies delivered intravenously (IV). PHP.eB encodes for a capsid originating from AAV9 that allows efficient AAV transit across the mouse blood brain barrier, which enables delivery of AAV vectors in a brain-wide fashion. This capsid differs from AAV9 such that amino acids starting at residue 586: SAQA are changed to SDGTLAVPFKA. The Gad2-T2A-nls-mCherry reporter marks nearly all inhibitory neurons in the mouse brain (here shown V1 visual cortex), and the delivered CN1244/PHP.eB virus drives specific SYFP2 reporter activity in forebrain inhibitory neurons.

FIGS. 4A, 4B. Comparison of CN1244 vs CN1389 vs CN1390. (FIG. 4A) Schematic representations of three vector constructs, CN1244, CN1389, and CN1390 (CN1203 scAAV). Key: hl56i—full-length human DIx156i enhancer (SEQ ID NO: 1); hl56iCore—human DIx156i enhancer core (SEQ ID NO: 2); minBG—minimal beta globin promoter; SYFP2—super yellow fluorescent protein 2; WPRE3—woodchuck hepatitis virus posttranscriptional regulatory element 3; BGHpA—bovine growth hormone polyA sequence; L-ITR and R-ITR-Adeno—associated virus-2 (AAV2) inverted terminal repeats (ITRs). (FIG. 4B) Fluorograph images showing relative expression of the SYFP2 from AAV vector constructs CN1244, CN1389, and CN1390.

FIG. 5. CN1390 retains cell type specificity of reporter expression for the pan-GABAergic neuronal populations (marked with red fluorescence in these Ai75 het/Gad2-IRES-Cre mice) in the juvenile mouse cortex and hippocampus in slice culture. 10 DIV/10 DPI.

FIGS. 6A-6E. CN1390 exhibits rapid onset of transgene expression in human ex vivo brain slices. (FIG. 6A) Time course of virus-mediated YFP expression following human brain slice transduction with CN1390/PHP.eB (left panel: 1 DIV/1 DPI; middle panel: 3 DIV/3 DPI; right panel: 6 DIV/6 DPI). (FIG. 6B) Expanded view of the boxed region in (FIG. 6A, right panel). (FIG. 6C) High magnification view of a virus labeled interneuron with bipolar morphology. (FIG. 6D) Example whole cell recordings from four different virus-labeled YFP+human interneurons and demonstrating diverse firing patterns to supra-threshold current injection. (FIG. 6E) Functional analysis of human neocortical interneuron firing patterns and electrical properties by patch clamp recording was feasible as early as 40 hours post-infection with CN1390/PHP.eB virus.

FIGS. 7A-7F. AAV vector reagents to reverse DS symptoms in Scn1 at/mice. (7A) Vectors to deliver epitope-tagged Nav genes of bacterial origin (NavBacs). The Nav genes shown here are NavMs (from Magnetococcus marinus), NavBp (from Bacillus pseudofirmus), and NavSheP-D60N (from Shewanella putrifaciens with an engineered D60N mutation). These examples all have N-terminal epitope tags (hexahistidine in the case of CN1367, or 3×HA for CN1498, CN1499, and CN1500). hl56i refers to the full-length 156i enhancer of SEQ ID NO: 1; 3×hl56iCore refers to the concatemerized core of the 156i enhancer (SEQ ID NO: 3); (7B) Graded expression levels from NavBac vectors. (7C) Weak but detectable expression in few Pvalb interneurons from vector CN1367. (7D) Trend towards seizure protection with vector CN1367. (7E) Vector 1500 drives high-level expression in Pvalb+ and Pvalb interneurons throughout cortex. (7F) Abundant production of HA-tagged NavBacs in cell bodies and proximal processes with vectors 1498 and 1500, but not 1499.

FIG. 8. Four exemplary strategies to encode and deliver full-length human SCN1A protein despite exceeding the AAV packaging limit. The human SCN1A gene (hSCN1A) has a 6030 bp open reading frame which exceeds the 4.7 kb packaging limit in AAV vectors. One approach to overcome this size limit is to split hSCN1A into two parts for delivery. Strategies 1 and 2 take advantage of homology-driven recombination between two separate AAV vector genomes (homologous regions indicated with hashed lines) to enable full open reading frame reconstitution in co-transduced cells. Strategies 3 and 4, in contrast, add trans-splicing elements (synthetic intron indicated by dots) to potentially increase the efficiency of open reading frame reconstitution and hence full-length protein expression. Strategies 1 and 3 utilize SYFP-P2A- and 3×HA-tagged hSCN1A protein to enable easy detection of both parts of the protein by fluorescence and immunohistochemistry, while strategies 2 and 4 utilize untagged hSCN1A protein and a vertebrate promoter to ensure low immunogenicity of the delivered transgene and encoded proteins. Another strategy is to utilize multiple vectors, each encoding a different segment or subunit of the SCN1A protein. Protein subunits can then self-assemble after expression naturally or through the inclusion of engineered cysteines or other linking domains.

FIG. 9. Full-length human SCN1A expression driven by a two-part AAV viral vector system. (Top panel): Model of two-part vector system. The top vector (1504) includes the concatemerized 3×hl56iCore (SEQ ID NO: 3) and promoter (minCMV) elements to drive expression in all inhibitory neurons (including both Pvalb+ and Pvalb-inhibitory neurons). The driven transgene includes a SYFP2 linked by a P2A tag to the N-terminal region of human SCN1A, which includes 604 bp homology to the C-terminal region included on the bottom vector 1512. The bottom vector 1512 also includes a C-terminal 3×HA tag and 3′UTR regulatory sequences (WPRE3 and polyA sites). (Bottom panel): Both AAV viral vectors were packaged into viral particles with PHP.eB capsid and both were delivered intravenously to a C57BI/6 mouse. After 21 days the mouse brain was harvested, fixed, and processed for immunofluorescence with anti-GFP (targeting SYFP2), anti-HA, and anti-Pvalb antibodies to detect transgene-expressing cells and their overlap with Pvalb+inhibitory neurons (which are cells having particular importance in DS symptomology). Many inhibitory neurons were labeled with the N-terminal SYFP2⁺ tag at high level, including both Pvalb+ and Pvalb inhibitory neurons. Some of these SYFP2⁺ cells also express HA which indicates fully intact SCN1A protein product being expressed in these GFP⁺HA⁺ cells (arrows). The vertical arrows indicate Pvalb⁺ interneurons expressing human SCN1A and the horizontal arrows indicate Pvalb interneurons expressing human SCN1A. These SCN1A-expressing cells are found throughout the forebrain (both neocortex and hippocampus [here dentate gyrus]), which includes the brain region known to be important in many epilepsies and known to be dysfunctional in DS.

FIG. 10. CN1500 rAAV vector substantially reverses febrile seizures in Scn1a^{+/-} mice. Febrile seizure assay shown as internal temperature where a seizure is first detected. (Top) Circles show Scn1a^{+/-} mice untransduced with AAVs, while the diamonds represent animal that were transduced with CN1500. The large dot and error bars represent the average+/−SEM for each group of animals. (Bottom) Trends of the same data are shown as the percentage of mice in each group that remain seizure free at different temperatures using a Kaplan-Meier curve.

FIG. 11. Exemplary sequences supporting the disclosure.

### DETAILED DESCRIPTION

In vertebrates, voltage gated sodium channels (Navs) are heteromeric protein complexes including a large central pore composed of alpha subunits, and smaller auxiliary subunits composed of beta subunits which modulate the kinetics and subcellular distribution of the pores. The alpha subunits are encoded by a family of 9 different genes (from SCN1A to SCN11A), and the beta subunits are encoded by 4 different genes (SCN1B to SCN4B), and these gene members show cell- and tissue-specific expression patterns. Each of the nine different alpha subunit genes seeds a distinct Nav channel complex, making 9 different subtypes (Nav1.1-Nav1.9), and these nine Nav channels display tissue specific localization and functional differences (See, Goldin, (2001) Annu Rev Physiol 63:871-94; and Yu et al., (2003), J. Neurosci 23:7577-758).

Navs are central for neuron function, being responsible for initiating the rapid upstroke of action potentials in excitable nervous system cells, and Nav1.1 drives this activity in many cells. The Nav1.1 channel includes the SCN1A alpha subunit, and is expressed in nearly all neurons and at high levels in GABAergic neurons. The Nav1.2 channel includes the SCN2A subunit; the Nav1.3 channel includes the SCN3A subunit, the Nav1.4 channel includes the SCN4A subunit, the Nav1.5 channel includes the SCN5A subunit, the Nav1.6 channel includes the SCN8A subunit, the Nav1.7 channel includes the SCN9A subunit, the Nav1.8 channel includes the SCN10A subunit, and the Nav1.9 channel includes the SCN11A subunit.

There are numerous neurological disorders for which treatments are urgently needed. One class of such disorders arise due to dysfunctional Nav1.1 sodium channels in inhibitory neurons. For example, the following disorders and conditions are associated with dysfunctional Nav1.1 sodium channels: epilepsy (including Dravet syndrome (DS), generalized epilepsy with febrile seizures plus (GEFS+), borderline DS, intractable childhood epilepsy with generalized tonic-clonic seizures (ICEGTC), cryptogenic focal and generalized epilepsies, myoclonic-astatic epilepsy (Doose syndrome), Lennox Gastaut syndrome, and severe infantile multifocal epilepsy (Gambardella A et al. Epilepsia. 2009, 50 Suppl 5:20-3)), West syndrome, also known as infantile spasms (Harkin L. A. et al. Brain, 2007, 130(3) 843-852), rare cases of familiar migraine such as familial hemiplegic migraine 3 (FHM3), Panayiotopoulos syndrome (Livingston J. H. et al. J Child Neurol. 2009, 24(4): 503-8), familial autism (Weiss L. A et al. 2003, Molecular Psychiatry, 8(2), 186-194), sporadic autism spectrum disorders (ASDs) (Nat Genet. 2011 43(6): 585-9), Rasmussens's encephalitis, also known as chronic focal encephalitis, or CFE (Ohmori I. et al, Epilepsia. 2008 49(3): 521-6), Alzheimer's disease (Scharfman H. E. Epilepsy Curr. 2012 12(5): 178-183), and cerebral ischemia-reperfusion (Yao C. et al, Neurotox Res. 2002; 4(1): 67).

The underlying cause of epilepsy is believed to arise from a defect in the excitation-inhibition (E/I) balance of cortical circuits. Forebrain GABAergic interneurons are the primary source of inhibition in the telencephalon and various lines of evidence indicate their importance in epilepsy. DS particularly is a severe childhood epilepsy predominantly due to SCN1A haploinsufficiency. It is marked by severe and frequent seizures (sometimes hundreds a day), leads to developmental delay, and has a lethality rate of up to 16%. DS affects nearly 1 in 20,000 births in both the United States and Europe, and more than 10,000 people are estimated to suffer from DS on both continents (Wu et al., Pediatrics 136, e1310-e1315, 2015). Heterozygous loss-of-function mutations in SCN1A, the gene that encodes the pore forming subunit of the voltage-gated sodium channel Nav1.1 is the predominant cause of DS (Catterall et al., J. Physiol. 588, 1849-1859, 2010; Claes et al., Hum. Mutat. 21, 615-621, 2003; Fujiwara, Epilepsy Res. 70 Suppl 1, S223-230, 2006; Verbeek et al., Epilepsy Behav. EB 47, 39-44, 2015). Genetic models of DS have established the pathophysiology of this disease (Han et al., Nature 489, 385-390, 2012; Kalume et al., J. Clin. Invest. 123, 1798-1808, 2013; Kalume et al., J. Neurosci. 27, 11065-11074, 2007; Kalume et al., Neurobiol. Dis. 77, 141-154, 2015; Oakley et al., Proc. Natl. Acad. Sci. 106, 3994-3999, 2009; Cheah et al., Proc. Natl. Acad. Sci. 109, 14646-14651, 2012; Han et al., Proc. Natl. Acad. Sci. 109, E368-E377, 2012). Mouse models of DS have reduced Nav1.1 function due to heterozygous deletion of Scn1a and exhibit the key phenotypic traits of DS including: febrile seizures, anxiety, and sleep deficits (Han et al., Nature 489, 385-390, 2012; Kalume et al., J. Clin. Invest. 123, 1798-1808, 2013; Kalume et al., Neurobiol. Dis. 77, 141-154, 2015; Oakley et al., Proc. Natl. Acad. Sci. 106, 3994-3999, 2009; Tai et al., Proc. Natl. Acad. Sci. 111, E3139-E3148, 2014; Yu et al., Nat. Neurosci. 9, 1142-1149, 2006).

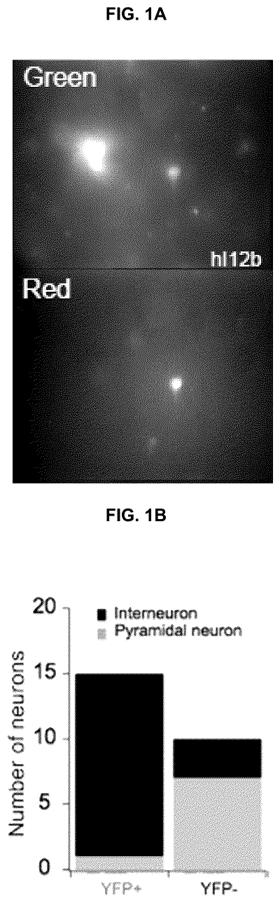

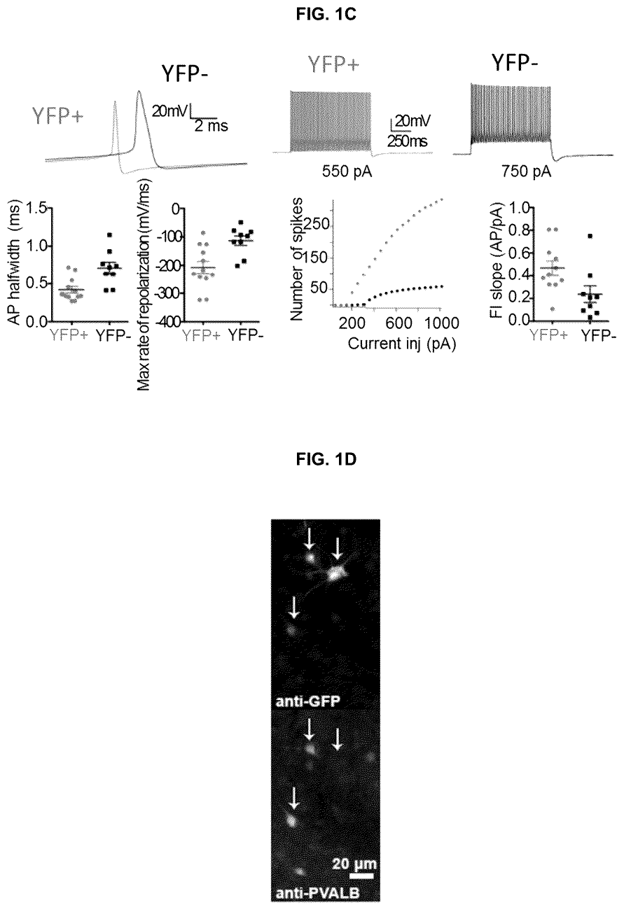

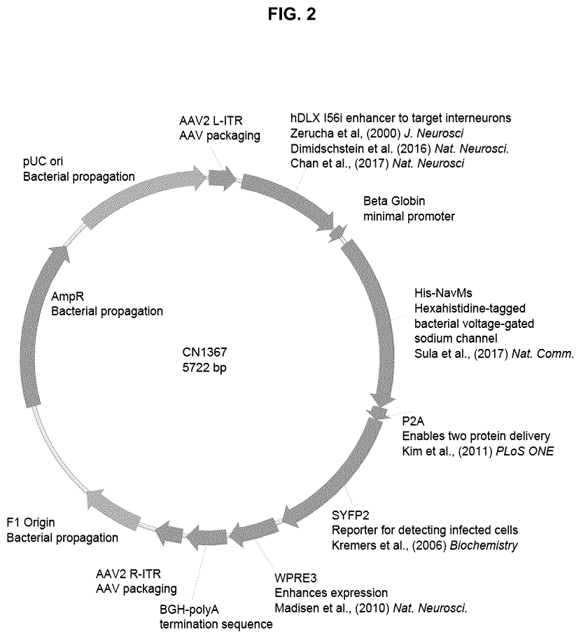

DS is a disease of forebrain interneurons. Global Scn1a deletion causes reduced sodium current and excitability of GABAergic interneurons with no detectable impact on excitatory neurons (Catterall et al., J. Physiol. 588, 1849-1859, 2010; Kalume et al., J. Neurosci. 27, 11065-11074, 2007; Cheah et al., Proc. Natl. Acad. Sci. 109, 14646-14651, 2012; Tai et al., Proc. Natl. Acad. Sci. 111, E3139-E3148, 2014; Yu et al., Nat. Neurosci. 9, 1142-1149, 2006; Ogiwara et al., J. Neurosci. 27, 5903-5914, 2007 Rubinstein et al., Brain 138, 2219-2233, 2015; Mistry et al., Neurobiol. Dis. 65, 1-11, 2014). Conditional deletion of Scn1a in forebrain or interneurons using specific Cre drivers qualitatively reproduced the key symptoms of DS, whereas excitatory neuron-specific deletion caused no detectable phenotype (Cheah et al., Proc. Natl. Acad. Sci. 109, 14646-14651, 2012). In addition, targeted deletion of Scn1a in Pvalb⁺ or Sst⁺ interneuron classes (separately or in combination) revealed that dysfunction in each class separately contributes to the multifaceted phenotypes of DS, with Pvalb+ cells exerting a greater effect (Rubinstein et al., Brain 138, 2219-2233, 2015; Dutton et al., Neurobiol. Dis. 49, 211-220, 2013). Consistent with these mouse models, patients with DS demonstrated a reduced GABAergic inhibition, and no change in glutamatergic neuron excitability following transcranial stimulation testing (Stern et al., Neurology 88, 1659-1665, 2017). Thus, studies of both mouse and human strongly indicate that DS is a disease of forebrain interneurons, caused by pathogenic loss-of-function mutations in SCN1A. Based on this insight, it was hypothesized that targeted introduction of functional voltage gated sodium channel with properties similar to Nav1.1 in forebrain GABAergic interneurons would improve or eliminate DS symptoms via rescue of their physiology. For exemplary methods to electrophysiologically and phenotypically characterize DS in a mouse model, see FIG. 1 of Rubinstein et al., Brain 138 (Pt 8): 2219-33, 2015.

Cell type- or cell class-specific gene delivery using non-pathogenic recombinant adeno-associated virus (rAAV) is showing increasing promise for the treatment of diverse diseases. Inclusion within rAAVs of one or more cis-acting DNA-control elements, such as specific promoters or enhancers, has been beneficial to provide specificity for expression within particular target cells, including specific cell types or cell classes in the brain. For example, Dimidschstein and colleagues (Nat Neurosci 19(12): 1743-1749, 2016) developed a rAAV that results in selective expression of a gene within GABAergic interneurons within the telencephalon. The rAAV includes a 529 base pair (bp) enhancer sequence (referred to as ml56i or mDIx) from the distal-less homeobox 5 and 6 (DIx5/6) genes, which are naturally expressed by forebrain GABAergic interneurons during embryonic development (Zerucha et al., J. Neurosci. 20(2): 709-721, 2000). A construct developed by Dimidschstein et al. is available on Addgene as ID #83900 (in which the enhancer drives eGFP expression). Additional constructs which employ the murine or human 156i enhancer to drive various transgenes are also available through Addgene, such as Plasmid ID #s 83899 (driving GCaMP6f expression), 83898 (driving ChR2-mCherry expression), 83895 (driving Cre recombinase-dependent eGFP expression), 89897 (driving bicistronic hM3Dq and nls-dTomato expression), 83896 (driving bicistronic hM4Di and nls-dTomato expression), and 83894 (driving cre recombinase-dependent tdTomato expression). See also U.S. Patent Publication No. US2018/0078658. Additionally, the mDIx enhancer has previously been used to reliably target reporter genes in a pattern very similar to the normal patterns of DIx5/6 expression during embryonic development (Zerucha et al., J Neuroscience 20:709-721, 2000; Stühmer et al., Cerebral Cortex 12:75-85, 2002; Stenman et al., J Neuroscience 23:167-174, 2003; Monory et al., Neuron. 51:455-455, 2006; Miyoshi et al., J Neuroscience 30:1532-1594, 2010).

One significant drawback to using rAAVs as a selective gene-delivery system is the strictly restricted packaging limit of AAVs; this is particularly limiting to the inclusion of lengthy genetic control elements. In addition, existing interneuron-specific rAAV expression constructs provide weak expression in certain applications, as well as for expression of transgenes (such as therapeutic genes) that are more poorly tolerated than GFP. Thus, there remains a need in the art for even shorter enhancer sequences that are capable of providing cell-specific expression of Nav1.1 function-restoring proteins particularly in neurons such as inhibitory interneurons. There also remains a need for genetic elements that provide stronger expression, and/or that work with a wider selection of reporter or other target genes and in a wider array of expression contexts.

The current disclosure provides expression constructs that result in high levels of protein expression in inhibitory neurons for the purpose of rescuing voltage-gated sodium channel function. The protein expression is selective to targeted inhibitory neurons and results in therapeutic efficacy to treat DS in a well-established in vivo mouse model of the disease, particularly that described in Kalume et al., J. Clin. Invest. 123, 1798-1808, 2013; and Oakley et al., Proc. Natl. Acad. Sci. 106, 3994-3999, 2009. In this model, mice can be implanted with electroencephalography (EEG) and electromyography (EMG) electrodes using approaches described in Kalume et al., J. Clin. Invest. 123, 1798-1808, 2013; Kalume et al., Neurobiol. Dis. 77, 141-154, 2015; and Oakley et al., Proc. Natl. Acad. Sci. 106, 3994-3999, 2009). After recovery, animals can be continuously monitored by video, EEG, and EMG. During testing procedures, mouse body temperature can be increased in 0.5° C. steps until either a generalized tonic-clonic seizure occurs, or a core body temperature of 42° C. is achieved. For each treatment group, a Kaplan Meier curve for seizure susceptibility due to temperature can be generated and the average temperature of seizure occurrence can be computed. Chi-squared tests can contrast Kaplan Meier plots of seizure susceptibility, and unpaired 2-tailed t-tests ascertain differences among average temperatures of seizure occurrence.

In particular embodiments, the expression constructs include non-naturally occurring enhancer element sequences that demonstrate strong and rapid interneuron-specific expression when used to drive a heterologous encoding sequence to treat Nav1.1 associated sodium channel disorders. In particular embodiments, the non-naturally occurring enhancer element includes multiple copies of a shortened, core portion of the human or murine 156i enhancer (SEQ ID NO: 3). In fact, the present disclosure can include a concatemerized core of the 156i enhancer from any species, so long as the concatemerized core results in selective expression of a functional protein in inhibitory neurons. For example, the Zebrafish 156i enhancer is provided as SEQ ID NO: 5 and the core of the Zebrafish 156i enhancer is provided as SEQ ID NO: 6. Particular embodiments provide a non-naturally occurring enhancer element including a three-copy concatemer of the human, murine, or zebrafish 156i core, such as shown in SEQ ID NO: 3 and SEQ ID NO: 7. Additional embodiments include 2×, 4×, 5×, 6×, 7×, 8×, 9×, or 10× copies of 156i core sequence of SEQ ID NO: 2 or SEQ ID NO: 6, arranged for instance in tandem. Particular embodiments can include a concatemerized hybrid of SEQ ID NO: 2 and SEQ ID NO: 6 (e.g., 2-6-2, 6-2-6, 2-2-6, 6-6-2).

In particular embodiments, a concatemerized core of the 156i enhancer is used to minimize the size required to enhance expression of proteins or nucleotide sequences that rescue voltage-gated sodium channel function. The synthetic 3× human DLX 156i core enhancer (a.k.a., 3×hl56iCore; SEQ ID NO: 3) is shorter than the original full-length enhancer sequence reported in Dimidschstein et al. (Nat Neurosci 19(12): 1743-1749, 2016), despite being a 3× concatemer of the strongly conserved core of the enhancer. When used to construct a heterologous expression cassette driving expression of proteins rescuing Nav1.1 sodium channel function, this concatemerized core enhancer provided more room for cargo genes linked to the enhancer, which is highly desirable in gene therapy vectors.

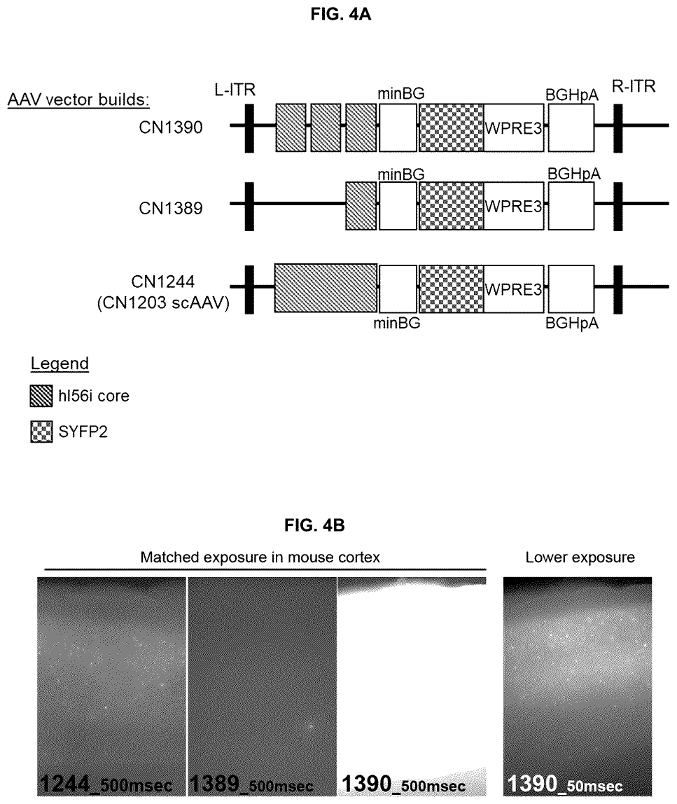

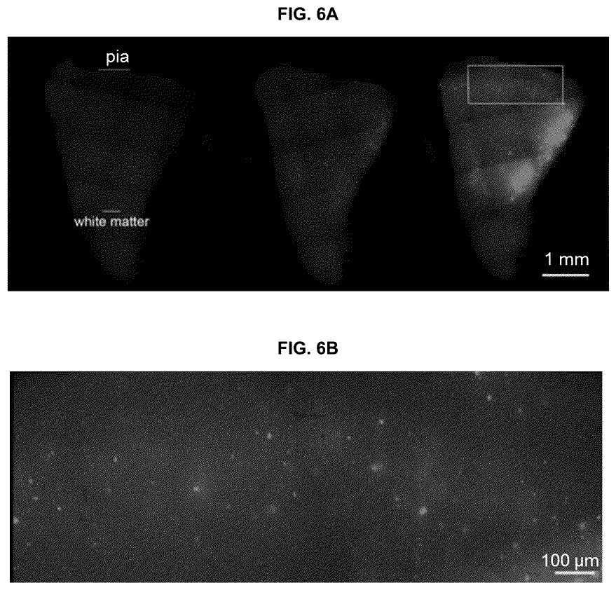

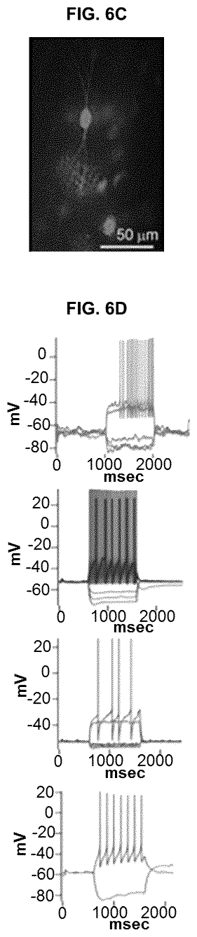

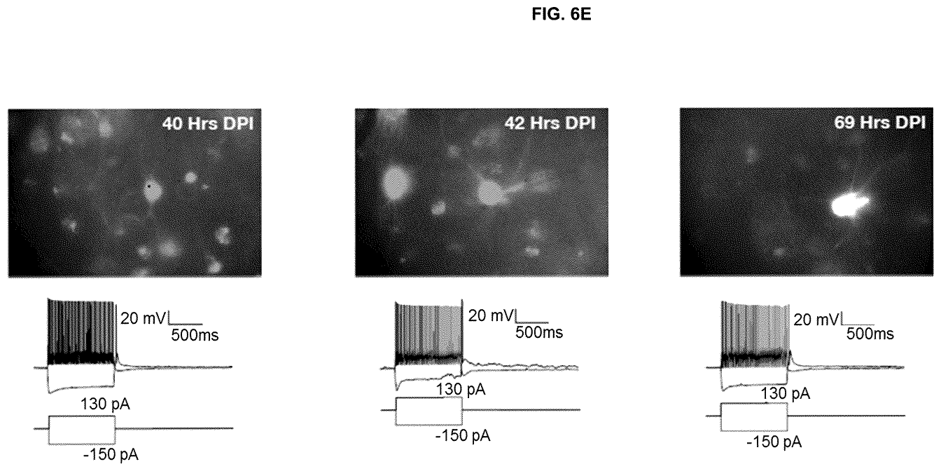

The compact size of the enhancer core, together with the multiple copies linked together as a concatemer, led to unexpectedly strong peak transgene expression in forebrain interneurons following viral transduction of mouse and human brain tissue (see FIGS. 4B & 5). The onset is also surprisingly rapid (see FIGS. 6A, 6B), leading to faster and higher expression in direct comparison to virus packaged with, for instance, the enhancer of Addgene plasmid #83900. The increase in expression is supra-linear and not simply three times the level driven by the full-length enhancer (SEQ ID NO: 1). Thus, the concatemerized 156i core enhancer also enables new and improved viral (and other) vectors such as those described herein with demonstrably better performance in the treatment of Nav1.1 sodium channel disorders. These are particularly useful for achieving transgene expression in inhibitory brain cell types such as neocortical GABAergic interneurons in diverse animal species.

Thus, the current disclosure provides expression constructs, vectors, and methods useful in reversing or ameliorating deficiency of function of the voltage-gated sodium channel Nav1.1. In particular embodiments, the current disclosure provides treatment of Nav1.1 channel disorders by selectively delivering a gene providing a protein or nucleotide sequence that allows for rescue of voltage-gated sodium channel function to inhibitory interneurons with impaired Nav1.1 activity.

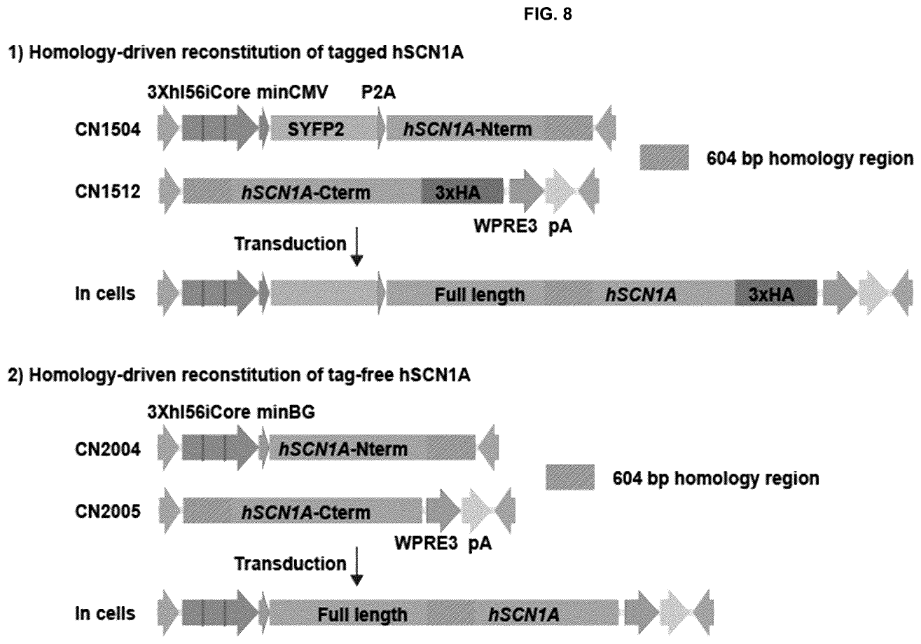

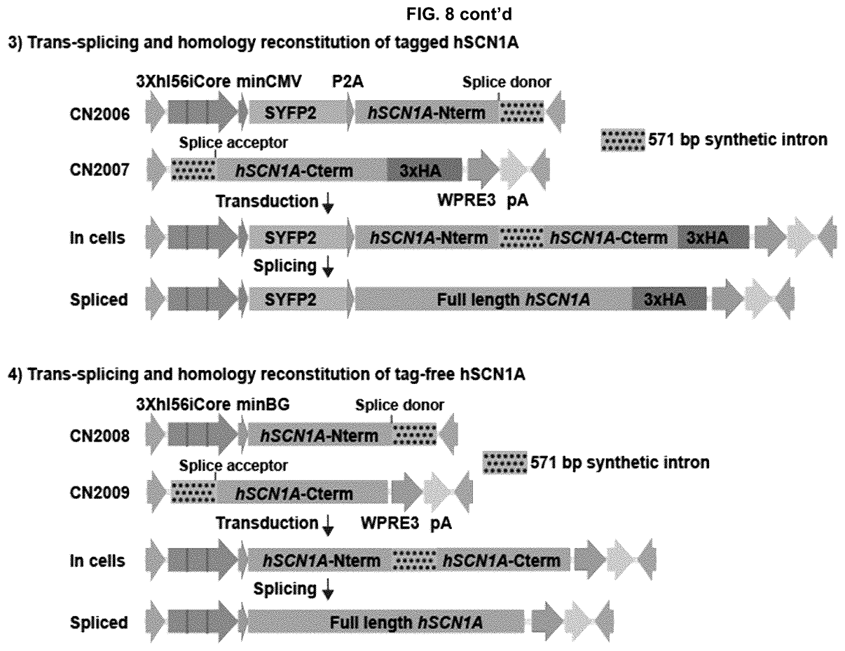

In particular embodiments, genes encoding proteins that rescue voltage-gated sodium channel function when Nav1.1 is impaired, are Nav genes of bacterial origin (NavBac) that function in human cells (Nguyen et al., Nat. Commun. 7, 13132, 2016; Sula et al., Nat. Commun. 8, 14205, 2017; DeCaen et al., eLife 3, e04387, 2014). Particular exemplary NavBacs include NavSheP, NavBp, and NavMs, which are three Nav proteins from three separate bacterial species. For representative experimental protocols and data demonstrating that bacterial voltage-gated sodium channels can confer sodium conductance to non-excitable cells, see Nguyen et al., Nature Communications 18(7), 13132, 2016 and particularly Supplemental FIG. 8.

NavBac proteins can also be engineered by mutagenesis to increase or alter activity as necessary, as in NavSheP-D60N. Additionally, these proteins can be engineered to incorporate tags or other fusion proteins for detection, as in His-NavMs (which contains an N-terminal hexahistidine tag), or with N-terminal 3×HA tags, or with other epitope tags or fluorescent protein tags or with any other protein tag as necessary. In particular embodiments, expression constructs do not encode immunogenic components. In particular embodiments, expression constructs do not include or encode immunogenic components.

In particular embodiments, the genes encoding proteins that restore voltage-gated sodium channel function are genes encoding SCNA1 (e.g., human SCNA1 or mouse Scna1).

In particular embodiments, therapeutic treatments are based on the intravenous, retro-orbital, intraspinal, and/or intrathecal administration of viral vectors result in selective expression within targeted inhibitory neurons.

In particular embodiments, provided herein is an AAV viral vector CN1500, a recombinant AAV that expresses the transgene SYFP2-P2A-NavSheP-D60N, which when translated is cleaved into two proteins: SYFP2 (reporter) and NavSheP-D60N. As indicated, NavSheP-D60N (Nguyen et al., Nat. Comm. 7:13132, 2016) is a modified voltage-gated sodium channel of bacterial origin that has been modified to improve the channel kinetics and codon-optimized for increased expression in mammalian cells. The transgene expression level is elevated by the addition of a WPRE3 element, and transcription is terminated with the bovine growth hormone poly adenylation sequence (BGHpA). Expression of the transgene is high and limited to inhibitory cells in forebrain structures including the cortex and the hippocampus, via the 3×hl56iCore synthetic enhancer directly 5′ of a cytomegalovirus (CMV) minimal promoter. Furthermore, the therapeutic transgene NavSheP-D60N can be labeled by an HA epitope tag to verify protein expression and correct protein localization. Therapeutic efficacy in a mouse model of DS has been demonstrated. In particular embodiments, the HA epitope tag can be removed from CN1500 and other vector designs described herein.

The human SCNA1 gene is relatively large and can benefit from administration from a vector that accepts a larger cargo than AAV or by using a two-AAV vector administration strategy. Multiple different strategies can be utilized in order to split a large open reading frame among two AAV vectors. Four strategies to split the large hSCN1A open reading frame are depicted in FIG. 8, but several more can be imagined.

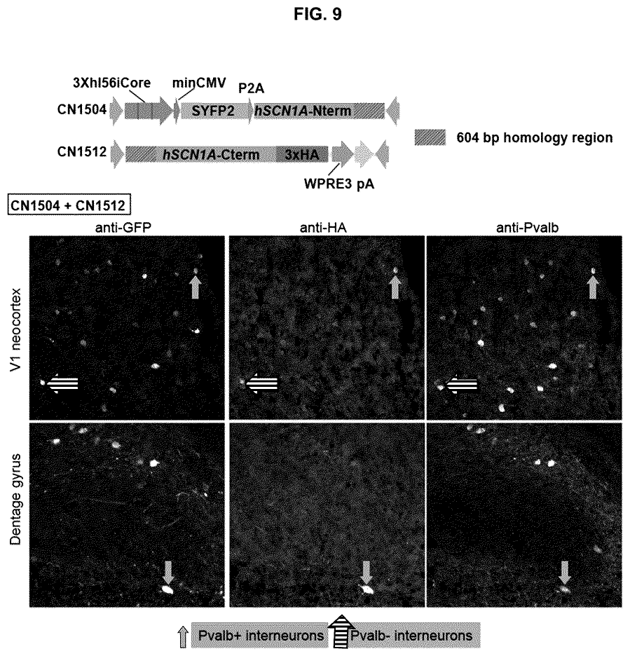

In particular embodiments, a two-vector administration strategy can split a coding sequence between two vectors. In particular embodiments, the first vector can encode the N-terminal portion of a protein and include a promoter and an enhancer. The second vector can encode the C-terminal portion of the protein and include a termination signal and a polyA signal. The portions of the protein encoded by each vector overlap to create a region of homology. Example 2, FIG. 8 strategy 1, and FIG. 9 show an example of such a two-vector system that achieved selective expression of the human SCN1A protein in inhibitory neurons. The 604-base pair (bp) region of homology is depicted in FIG. 9 by diagonal hashing. However, as is understood by one of ordinary skill in the art, a wide range of number of homologous bp can be selected. In particular embodiments, bp regions of homology range from less than 75 to more than 1500 bp. Moreover, various other components and configurations can be appropriate in a two-vector system as described in relation to single vector systems in more detail elsewhere herein. Another possible configuration includes a synthetic intron element that provides a region of homology as well as splice donor and acceptor sites that make final reconstitution of the protein more efficient and lead to higher levels of protein expression.

Moreover, additional strategies can also be used to provide exogenous voltage-gated sodium channel activity to inhibitory neurons that are deficient in this activity. For example, particular embodiments can utilize expression constructs encoding an artificial transcription factor that increases the expression of the endogenous functional copy of SCN1A.

In particular embodiments, expression constructs can encode molecules that increase the prevalence of functional SCN1A mRNA molecules, for example through post-transcriptional positive regulation of splicing and or stability.

In particular embodiments, strategies can be employed that restore the full length hSCN1A transcript through trans-splicing the hSCN1A transcript separated into two parts.

In particular embodiments, expression constructs (e.g., AAV) can deliver hSCN1A protein as several (two or four) subunit ORFs delivered by two or four separate vectors.

Aspects of the disclosure are now described with the following additional options and detail: (i) Expression Constructs & Vectors; (ii) Compositions for Administration (iii) Methods of Use; (iv) Kits and Commercial Packages; (iv) Exemplary Embodiments; and (v) Experimental Examples.

(i) Expression Constructs & Vectors. Expression constructs disclosed herein include (i) a concatemerized core of the 156i enhancer sequence that leads to selective expression of a coding sequence within inhibitory neurons, (ii) a coding sequence that is expressed and results in a protein or nucleotide sequence that rescues voltage-gated sodium channel function in a cell in need thereof, and (iii) a promoter. The expression construct can also include other regulatory elements if necessary or beneficial. In particular embodiments, expression constructs are isolated polynucleotides.

In particular embodiments, an “enhancer” or an “enhancer element” is a cis-acting sequence that increases the level of transcription associated with a promoter, and can function in either orientation relative to the promoter and the coding sequence that is to be transcribed, and can be located upstream or downstream relative to the promoter or the coding sequence to be transcribed. There are art-recognized methods and techniques for measuring function(s) of enhancer element sequences. By way of example, specific methods for determining or measuring function(s) of a 156i enhancer are described in Dimidschstein et al. (Nat Neurosci 19(12): 1743-1749, 2016) and U.S. Patent Publication No. US2018/0078658. Particular examples of enhancer sequences include the human full-length 156i enhancer (SEQ ID NO: 1), the hl56 core (SEQ ID NO: 2), and the 3×hl56i core (SEQ ID NO: 3) as well as the murine and zebrafish orthologs thereof.

In particular embodiments, an inhibitory-neuron-specific enhancer is an enhancer that is uniquely or predominantly utilized in inhibitory neurons. An inhibitory-neuron-specific enhancer enhances expression of a gene in inhibitory neurons, but does not substantially affect expression of genes in other cell types, for example non-inhibitory neurons or glial cells, thus having neuronal specific transcriptional activity. In some instances there may be some low level expression in other cell types, but such expression is substantially lower than in inhibitory neurons, for example less than 1% or 1%, 2%, 3%, 5%, 10%, 15% or 20% of the expression levels in inhibitory neurons. In particular embodiments, interneurons are the only cell types that express the right combination of transcription factors that bind to the concatemerized core of the 156i enhancer to drive gene expression.

In particular embodiments, selective expression within inhibitory neurons is demonstrated by 10% more expression, 20% more expression, 30% more expression, 40% more expression; 50% more expression; 60% more expression, 70% more expression, 80% more expression, 90% more expression, 100% more expression or more over non-inhibitory neurons. In particular embodiments, selective expression within inhibitory neurons is demonstrated by expression within inhibitory neurons and no detectable expression within non-inhibitory neurons.

In particular embodiments, selective expression within GABAergic interneurons is demonstrated by 10% more expression, 20% more expression, 30% more expression, 40% more expression; 50% more expression; 60% more expression, 70% more expression, 80% more expression, 90% more expression, 100% more expression or more over non-GABAergic interneurons. In particular embodiments, selective expression within GABAergic interneurons is demonstrated by expression within GABAergic interneurons and no detectable expression within non-GABAergic interneurons.

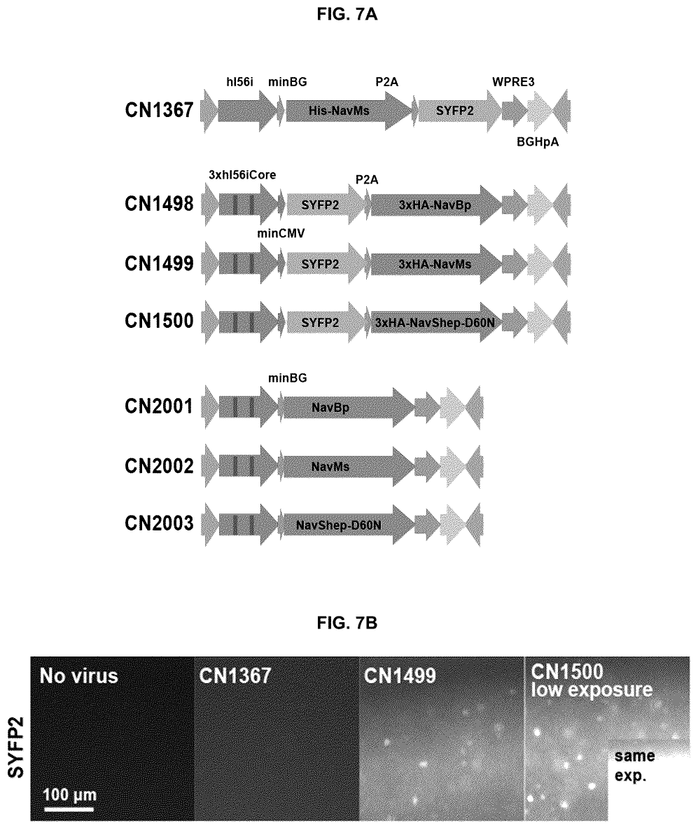

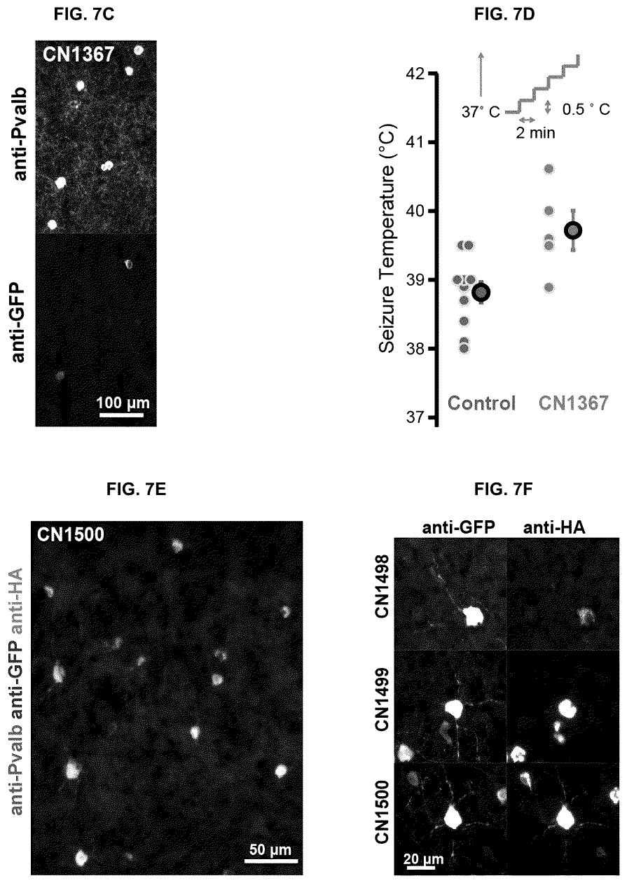

In particular embodiments, one class of inhibitory neurons can be identified based on Pvalb expression as described in relation to FIGS. 1, 7C, 7E, and 8. In particular embodiments, GABAergic interneurons can be distinguished from other cell types by the expression of the genes Gad2 and Gad1; in the adult cortex, GABAergic interneurons can be distinguished from glutamatergic excitatory neurons by the presence of GABA. In the adult striatum, GABAergic interneurons can be distinguished from Medium Spiny Neurons by the expression of the gene Nkx2.1. (See, for example, Rudy et al., Devel Neurobio 71, 45-61(2011)): Kepecs. & Fishell, Nature. 505, 318-326(2014).

In particular embodiments, a coding sequence encodes a protein or nucleotide sequence that rescues voltage-gated sodium channel function. In particular embodiments, the coding sequence further encodes a reporter protein. If the coding sequence encodes a protein or nucleotide sequence that rescues voltage-gated sodium channel function and a reporter protein, it can further encode a skipping element such as a self-cleaving peptide or an internal ribosome entry site (IRES) sequence.

Exemplary proteins that restore voltage-gated sodium channel function include NavShep, NavShep-D60N, NavBp, NavMs, and hSCN1A, and their epitope-tagged variants 3×HA-NavShep-D60N, 3×HA-NavBp, 3×HA-NavMs, His-NavMs, and hSCN1A-3×HA.

Exemplary coding sequences that encode proteins that rescue voltage-gated sodium channel function include SEQ ID NOs: 9-16.

Further, as indicated above, there are additional strategies that can be used to provide exogenous voltage-gated sodium channel activity to inhibitory neurons that are deficient in this activity. For example, in particular embodiments, enhancer-vectors (e.g., AAV) can encode an artificial transcription factor that increases the expression of the endogenous functional copy of SCN1A (or other SCN_A family gene) in SCN1A+/patients (e.g., Dravet patients) or other patients deficient in other voltage-gated sodium channels. This artificial transcription factor can contain a specific DNA-binding domain linked to a general transcription-activating domain. The DNA-binding domain can be engineered from a Cas9-related gene using CRISPR activation technology (as in Matharu et al., 2019, Science, 363(6424), 186-194 wherein Cas proteins are engineered to lack nuclease activity), or from other custom DNA-sensing proteins such as TALE-transcription factors (TFs) (Morbitzer et al., 2010, Proc. Nat. Acad. Sci., 107(50), 21617-21622) or zinc finger TFs (Gersbach et al., 2014, Acc. Chem. Res, 47(8), 2309-2318). Additional information and options regarding CRISPR and other targeted gene binding (and optionally editing) systems and components are provided below.

In particular embodiments, enhancer-vectors can encode molecules that increase the prevalence of functional SCN1A mRNA molecules, for example through post-transcriptional positive regulation of splicing and or stability, eventually leading to increases in functional voltage-gated sodium channel activity. This could be accomplished through AAV introduction of antisense RNA oligonucleotide molecules to increase splicing (Hsiao et al., 2016, EBioMedicine. 9, 257-277), or to prevent potential microRNA negative regulation.

In particular embodiments, strategies can be employed that restore the full length hSCN1A transcript through trans-splicing the hSCN1A transcript separated into two parts. This can be accelerated through the use of strong synthetic splice donors and splice acceptors in a two vector system, and through the use of different ITRs (ITR2 and ITR5) that induce proper juxtaposition of the two different genomes in cell after transduction (McClements M E, et al., 2017, Yale J. Biol Med. 90(4): 611-623).

In particular embodiments, enhancer-vectors (e.g., AAV) can deliver hSCN1A protein as several (two or four) subunit ORFs delivered by two or four separate vectors. Nav channels display pseudo-four-fold internal symmetry, due to four similar internal domains within the alpha subunit (Shen et al., Science 363(6433), 1303-1308), demonstrating that this protein can be amenable to delivery in two or four pieces. As indicated previously, assembly of sub-units can occur naturally and/or can be facilitated by the inclusion of engineered cysteines or other linking domains.

In particular embodiments, due to its internal 4-domain pseudo-symmetry, hSCN1A can be delivered in four fragments encoded on four different vectors that can self-assemble into a tetramer that is functionally and chemically equivalent to the functional NaV1.1 alpha subunit, except with breakpoints in surface loops. Alternatively, a single fragment may be delivered on a single vector that may self-assemble into a homo-tetrameric complex resembling the NaV1.1 alpha subunit which consists entirely of human sequence. Either strategy can deliver human non-immunogenic biomolecules to inhibitory cells for the purpose of rescuing Nav1.1 channel function in neurons that are deficient in voltage-gated sodium channel activity.

Additional strategies could also be used to reduce the hSCN1A ORF into a smaller (e.g., AAV-compatible) size (<4.7 kb).

Exemplary reporter proteins include fluorescent proteins such as yellow fluorescent molecules such as SYFP2, Citrine, PhiYFP and ZsYellow1; red fluorescent molecules such as mCherry, mRuby, Jred, and AsRed2; green fluorescent molecules such as green fluorescent protein (GFP), enhanced green fluorescent protein (EGFP), avGFP, ZsGreen, and mAzamiGreen; orange fluorescent molecules such as mOrange and mKusabira-Orange; blue fluorescent molecules such as Sapphire, mKalama1, EBFP2 and Azurite; cyan fluorescent molecules such as Cerulean and mTurquoise; far red proteins such as mPlum and mNeptune.

GFP is composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostability and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted ¿), also known as its optical cross section of 9.13E-21 m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm.

Exemplary self-cleaving peptides include the 2A peptides which lead to the production of two proteins from one mRNA. The 2A sequences are short (e.g., 20 amino acids), allowing more use in size-limited constructs. Particular examples include P2A, T2A, E2A, and F2A. In particular embodiments, the expression constructs include an internal ribosome entry site (IRES) sequence. IRES allow ribosomes to initiate translation at a second internal site on a mRNA molecule, leading to production of two proteins from one mRNA.

Coding sequences encoding proteins described herein can be obtained from publicly available databases and publications. Coding sequences can further include various sequence polymorphisms, mutations, and/or sequence variants wherein such alterations do not affect the function of the encoded protein. The term “encode” or “encoding” refers to a property of sequences of nucleic acids, such as a vector, a plasmid, a gene, cDNA, mRNA, to serve as templates for synthesis of other molecules such as proteins.

The term “gene” may include not only coding sequences but also regulatory regions such as promoters, enhancers, and termination regions. The term further can include all introns and other DNA sequences spliced from the mRNA transcript, along with variants resulting from alternative splice sites. The sequences can also include degenerate codons of a reference sequence or sequences that may be introduced to provide codon preference in a specific organism or cell type.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include minBglobin, CMV, minCMV, SV40 immediately early promoter, and the Rous Sarcoma Virus (RSV) long-terminal repeat (LTR) promoter.

In particular embodiments, expression constructs are provided within vectors. The term vector refers to a nucleic acid molecule capable of transferring or transporting another nucleic acid molecule, such as an expression construct. The transferred nucleic acid is generally linked to, e.g., inserted into, the vector nucleic acid molecule. A vector may include sequences that direct autonomous replication in a cell, or may include sequences that permit integration into host cell DNA. Useful vectors include, for example, plasmids (e.g., DNA plasmids or RNA plasmids), transposons, cosmids, bacterial artificial chromosomes, and viral vectors.

Viral vector is widely used to refer to a nucleic acid molecule that includes virus-derived nucleic acid elements that facilitate transfer and expression of non-native nucleic acid molecules within a cell. The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

Adenovirus. “Adenovirus vectors” refer to those constructs containing adenovirus sequences sufficient to (a) support packaging of an expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In particular embodiments, adenovirus type 5 of subgroup C is the preferred starting material in order to obtain a conditional replication-defective adenovirus vector for use in particular embodiments, since Adenovirus type 5 is a human adenovirus about which a great deal of biochemical and genetic information is known, and it has historically been used for most constructions employing adenovirus as a vector.

As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replication, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and rare integration into genomic DNA, and because their capsids and genomes can be tailored to allow expression in selected cell populations. scAAV refers to a self-complementary AAV. rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

Retrovirus. Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include: Moloney murine leukemia virus (M-MuLV), Moloney murine sarcoma virus (MOMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In particular embodiments, HIV based vector backbones (i.e., HIV cis-acting sequence elements) can be used.

“Self-inactivating” (SIN) vectors refer to replication-defective vectors in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment, 3′ LTR is modified such that the U5 region is replaced, for example, with an ideal poly(A) sequence. It should be noted that modifications to the LTRs such as modifications to 3′ LTR, 5′ LTR, or both 3′ and 5′ LTRs, are also included in particular embodiments.

In particular embodiments, viral vectors include a TAR element. The term “TAR” refers to the “trans-activation response” genetic element located in the R region of lentiviral (e.g., HIV) LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly(A) tract. The R region is also defined as being flanked by the U3 and U5 regions. The R region plays a role during reverse transcription in permitting the transfer of nascent DNA from one end of the genome to the other.

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766). In particular embodiments, vectors include a posttranscriptional regulatory element such as a WPRE or HPRE. In particular embodiments, vectors lack or do not include a posttranscriptional regulatory element such as a WPRE or HPRE.

Elements directing the efficient termination and polyadenylation of a heterologous nucleic acid transcript can increase heterologous gene expression. Transcription termination signals are generally found downstream of the polyadenylation signal. In particular embodiments, vectors include a polyadenylation sequence 3′ of a polynucleotide encoding a polypeptide to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA or SV40pA. In particular embodiments, a preferred embodiment of an expression construct includes a terminator element. These elements can serve to enhance transcript levels and to minimize read through from the construct into other plasmid sequences.

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of encoded genes in mammalian cell, such as pUC or Bluescript plasmid series.

Particular embodiments include:

| Vector | |
|---|---|
| Name | Features |
| CN1367 | rAAV2: hl56i-minBglobin-His-NavMs-P2A-SYFP2-WPRE3-BGHpA |
| CN1244 | rAAV2: hl56i-minBglobin-SYFP2-WPRE3-BGHpA |
| CN1389 | rAAV2: 1xhl56iCore-minBglobin-SYFP2-WPRE3-BGHpA |
| CN1390 | rAAV2: 3xhl56iCore-minBglobin-SYFP2-WPRE3-BGHpA |
| CN1203 | scAAV: hl56i-minBglobin-SYFP2-WPRE3-BGHpA |
| CN1180 | scAAV: hl12b-minBglobin-SYFP2-WPRE3-BGHpA |
| CN1498 | rAAV2: 3xhl56iCore-minCMV-SYFP2-P2A-3xHA-NavBp-WPRE3-BGHpA |
| CN1499 | rAAV2: 3xhl56iCore-minCMV-SYFP2-P2A-3xHA-NavMs-WPRE3-BGHpA |
| CN1500 | rAAV2: 3xhl56iCore-minCMV-SYFP2-P2A-3xHA-NavSheP-D60N-WPRE3-BGHpA |
| CN1504 | rAAV2: 3xhlI56iCore-minCMV-SYFP2-P2A-hSCN1A-Nterm |
| CN1512 | rAAV2: hSCN1A-Cterm-3xHA-WPRE3-BGHpA |
| CN2001 | rAAV2: 3xhl56iCore-minBG-NavBp-WPRE3-BGHpA |
| CN2002 | rAAV2: 3xhl56iCore-minBG-NavMs-WPRE3-BGHpA |
| CN2003 | rAAV2: 3xhl56iCore-minBG-NavSheP-D60N-WPRE3-BGHpA |
| CN2004 | rAAV2: 3xhl56iCore-minBG-hSCN1AFrontEnd |
| CN2005 | rAAV2: hSCN1ABackEnd-WPRE3-BGHpA |
| CN2006 | rAAV2: 3xhl56iCore-minBG-SYFP2ns-P2A-hSCN1AFrontEnd-IntronBridge |
| CN2007 | rAAV2: IntronBridge-hSCN1ABackEnd-3xHA-WPRE3-BGHpA |
| CN2008 | rAAV2: 3xhl56iCore-minBG-hSCN1AFrontEnd-IntronBridge |
| CN2009 | rAAV2: IntronBridge-hSCN1ABackEnd-WPRE3-BGHpA |
| CN2026 | rAAV-3xhl56i(core)-minBG-hSCN1A_Fragment1-WPRE3-BGHpA |
| CN2027 | rAAV-3xhl56i(core)-minBG-hSCN1A_Fragment2-WPRE3-BGHpA |
| CN2028 | rAAV-3xhl56i(core)-minBG-hSCN1A_Fragment3-WPRE3-BGHpA |
| CN2029 | rAAV-3xhl56i(core)-minBG-hSCN1A_Fragment4-WPRE3-BGHp |

In particular embodiments viral vectors with capsids that cross the blood-brain barrier (BBB) are selected. In particular embodiments, AAV are modified to include capsids that cross the BBB. Examples of AAV with viral capsids that cross the blood brain barrier include AAV9 (Gombash et al., Front Mol Neurosci. 2014; 7:81), AAVrh.10 (Yang, et al., Mol Ther. 2014; 22(7): 1299-1309), AAV1R6, AAV1R7 (Albright et al., Mol Ther. 2018; 26(2): 510), rAAVrh.8 (Yang, et al., supra), AAV-BR1 (Marchio et al., EMBO Mol Med. 2016; 8(6): 592), AAV-PHP.S (Chan et al., Nat Neurosci. 2017; 20(8): 1172), AAV-PHP.B (Deverman et al., Nat Biotechnol. 2016; 34(2): 204), and AAV-PPS (Chen et al., Nat Med. 2009; 15:1215).

AAV9 is a naturally occurring AAV serotype that, unlike many other naturally occurring serotypes, can cross the BBB following intravenous injection. It transduces large sections of the central nervous system (CNS), thus permitting minimally invasive treatments (Naso et al., BioDrugs. 2017; 31(4): 317), for example, as described in relation to the ongoing clinical trials for the treatment of superior mesenteric artery (SMA) syndrome by AveXis (AVXS-101, NCT03505099) and the treatment of CLN3 gene-Related Neuronal Ceroid-Lipofuscinosis (NCT03770572). In particular embodiments, a representative AAV9 capsid protein sequence can include the AAV9 VP1 capsid protein sequence (UniProt Accession number Q6JC40, SEQ ID NO: 57).

AAVrh. 10, was originally isolated from rhesus macaques and shows low seropositivity in humans when compared with other common serotypes used for gene delivery applications (Selot et al., Front Pharmacol. 2017; 8:441) and is currently being evaluated in clinical trials LYS-SAF302, LYSOGENE, and NCT03612869.

AAV1R6 and AAV1R7, two variants isolated from a library of chimeric AAV vectors (AAV1 capsid domains swapped into AAVrh.10), retain the ability to cross the BBB and transduce the CNS while showing significantly reduced hepatic and vascular endothelial transduction.

rAAVrh.8, also isolated from rhesus macaques, shows a global transduction of glial and neuronal cell types in regions of clinical importance following peripheral administration and also displays reduced peripheral tissue tropism compared to other vectors.

AAV-BR1 is an AAV2 variant displaying the NRGTEWD (SEQ ID NO: 53) epitope that was isolated during in vivo screening of a random AAV display peptide library. It shows high specificity accompanied by high transgene expression in the brain with minimal off-target affinity (including for the liver) (Körbelin et al., EMBO Mol Med. 2016; 8(6): 609).

AAV-PHP.S (Addgene, Watertown, MA) is a variant of AAV9 generated with the CREATE method that encodes the 7-mer sequence QAVRTSL (SEQ ID NO: 54), transduces neurons in the enteric nervous system, and strongly transduces peripheral sensory afferents entering the spinal cord and brain stem.

AAV-PHP.B (Addgene, Watertown, MA) is a variant of AAV9 generated with the CREATE method that encodes the 7-mer sequence TLAVPFK (SEQ ID NO: 55). It transfers genes throughout the CNS with higher efficiency than AAV9 and transduces the majority of astrocytes and neurons across multiple CNS regions.

AAV-PPS, an AAV2 variant crated by insertion of the DSPAHPS (SEQ ID NO: 56) epitope into the capsid of AAV2, shows a dramatically improved brain tropism relative to AAV2.

In particular embodiments, a capsid that results in brain-wide transduction of inhibitory cells in a primate following administration (e.g., i.v. administration) is chosen. In particular embodiments, a capsid that results in widespread transduction of tissue and cell types impacted by the loss of Scn1a following administration is chosen.

Compositions for Administration. Expression constructs and vectors of the present disclosure (referred to herein as physiologically active components) can be formulated with a carrier that is suitable for administration to human or animal subjects. Physiologically active components within compositions described herein can be prepared in neutral forms, as freebases, or as pharmacologically acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human, and in particular embodiments, when administered intravenously.

In particular embodiments, compositions can be formulated for intravenous, intraocular, intravitreal, parenteral, subcutaneous, intracerebro-ventricular, intramuscular, intrathecal, intraspinal, oral, intraperitoneal, oral or nasal inhalation, or by direct injection to one or more cells, tissues, or organs.

Compositions may include liposomes, lipids, lipid complexes, microspheres, microparticles, nanospheres, and/or nanoparticles.

The formation and use of liposomes is generally known to those of skill in the art. Liposomes have been developed with improved serum stability and circulation half-times (see, for instance, U.S. Pat. No. 5,741,516). Further, various methods of liposome and liposome like preparations as potential drug carriers have been described (see, for instance U.S. Pat. Nos. 5,567,434; 5,552,157; 5,565,213; 5,738,868; and 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are widely suitable as both water- and lipid-soluble substances can be entrapped, i.e. in the aqueous spaces and within the bilayer itself, respectively.

Liposomes may also be employed for site-specific delivery of active agents by selectively modifying the liposomal formulation. Should specific targeting be desired, methods are available for this to be accomplished. For example, binding domains of antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types.

In addition to the teachings of Couvreur et al. (FEBS Lett. 84(2): 323-326, 1977; Crit Rev Ther Drug Carrier Syst. 5(1) 1-20, 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

Alternatively, the disclosure provides for pharmaceutically acceptable nanocapsule formulations of the physiologically active components of the present disclosure. Nanocapsules can generally entrap compounds in a stable and reproducible way (Quintanar-Guerrero et al., Drug Dev Ind Pharm 24(12): 1113-1128, 1998; Quintanar-Guerrero et al., Pharm Res. 15(7): 1056-1062, 1998; Quintanar-Guerrero et al., J. Microencapsul. 15(1): 107-119, 1998; Douglas et al., Crit Rev Ther Drug Carrier Syst 3(3): 233-261, 1987). To avoid side effects due to intracellular polymeric overloading, ultrafine particles can be designed using polymers able to be degraded in vivo. Biodegradable polyalkyl-cyanoacrylate nanoparticles that meet these requirements are contemplated for use in the present disclosure. Such particles may be made as described in Couvreur et al., J Pharm Sci 69(2): 199-202, 1980; Couvreur et al., Crit Rev Ther Drug Carrier Syst. 5(1) 1-20, 1988; zur Muhlen et al., Eur J Pharm Biopharm, 45(2): 149-155, 1998; Zambaux et al., J Control Release 50(1-3): 31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In particular embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al., Prog Retin Eye Res, 17(1): 33-58, 1998), transdermal matrices (U.S. Pat. Nos. 5,770,219 and 5,783,208) and feedback-controlled delivery (U.S. Pat. No. 5,697,899).

Supplementary active ingredients can also be incorporated into the compositions.

Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In particular embodiments, for administration to humans, compositions should meet sterility, pyrogenicity, and the general safety and purity standards as required by United States Food and Drug Administration (FDA) or other applicable regulatory agencies in other countries.

Methods of Use. In particular embodiments, a composition including a physiologically active component described herein is administered to a subject to result in selective expression of a protein or nucleotide sequence that rescues voltage-gated sodium channel function within inhibitory neurons in the subject. In particular embodiments, rescuing voltage-gated sodium channel function includes converting a subject's interneurons lacking a sufficient quantity and/or activity of Nav1.1 sodium channels, into interneurons that express a sufficient quantity of exogenous voltage-gated sodium channels and activity, in order to recover neuronal function and to prevent epileptiform circuit activity.

In particular embodiments, rescued voltage-gated sodium channel function is evidenced by one or more of an increase in sodium channel current in and/or the increased excitability of an inhibitory neuron genetically-modified by the physiologically active component. In particular embodiments, rescued voltage-gated sodium channel function is evidenced by one or more of an increase in sodium channel conductance in and/or the sodium channel influx in response to voltage depolarization of an inhibitory neuron genetically-modified by the physiologically active component. An increase can be at least a 10% increase, at least a 20% increase, at least a 30% increase, at least a 40% increase, at least a 50% increase, at least a 60% increase, at least a 70% increase, at least an 80% increase or at least a 90% increase. The output of inhibitory neurons can be measured using an electrophysiological method, such as a multi-electrode array or a patch-clamp.

In particular embodiments, the inhibitory neuron is an inhibitory interneuron, a GABAergic neuron, a GABAergic interneuron, a pan-GABAergic neuron, or an inhibitory neuron in the hippocampus or cortex.

In particular embodiments, rescued voltage-gated sodium channel function is evidenced by increased sodium current-dependent fast spiking in forebrain interneurons, for example, using a mouse model. In particular embodiments, rescued voltage-gated sodium channel function is evidenced by delayed or prevented temperature-induced seizing in a mouse model as described herein.

Particular embodiments include identifying a subject with reduced Nav1.1 sodium channel function in inhibitory neurons. Such subjects can be identified based on a diagnosis of a disorder associated with Nav1.1 sodium channel dysfunction. Such disorders include epilepsy, an SCN1A-related seizure disorder, simple febrile seizures (FS), GEFS+, DS, intractable childhood epilepsy with generalized tonic-clonic seizures (ICE-GTC), intractable infantile partial seizures, myoclonic-astatic epilepsy, Lennox-Gastaut syndrome (LGS), and infantile spasms. Intractable seizures (also referred to as “uncontrolled” or “refractory” seizures) are seizures that cannot be controlled to a satisfactory degree based on sound medical judgment with conventional treatments.

Regarding DS particularly, 80% of DS patients test positive for an SCN1A gene mutation, but the absence of an SCN1A mutation does not exclude a DS diagnosis. DS is associated with mutations in SCN1A (such as partial or total deletion mutations, truncating mutations and/or missense mutations e.g. in the voltage or pore regions S4 to S6), SCN1B (encoding the sodium channel β1 subunit), SCN2A, SCN3A, SCN9A, GABRG2 (encoding the γ2 subunit of GABA receptor), GABRD (encoding the delta subunit of GABA receptor) and/or PCDH19 genes.

In particular embodiments, a subject in need of a treatment described herein may not experience diagnosable seizures, but exhibits subclinical electrical discharges, which refers to a high rate of seizure-like activity when their brain waves are measured with an electroencephalogram. Epileptic syndromes associated with these seizure-like discharges include Landau-Kleffner Syndrome, and Continuous Spike-wave Activity during Slow-wave Sleep.

In particular embodiments, patients may have an intellectual developmental disability (IDD) such as an Autism Spectrum Disorders (ASD). In particular embodiments, the patient of the disclosed method has epilepsy and an IDD or ASD disorder. Common IDD and ASD that are comorbid with seizures and epilepsy include fragile X syndrome (FXS), Rett syndrome (RTT), Angelman syndrome, Prader-Willi syndrome, Velocardiofacial syndrome, Smith-Lemli-Opitz syndrome, neuroligin mutations and “interneuronopathies” resulting from aristaless-related homeobox, X-linked (ARX) and Neuropilin 2 (NRP2) gene mutations.

The methods described herein may be particularly useful for treating children and infants, and for treating disorders that onset during infancy or childhood. In particular embodiments, the patient of the disclosed method is a newborn, a baby, a toddler, a preschooler, a school-age child, a tween, or a teenager. In particular embodiments, the patient is 18 years old or younger, 12 years old or younger, 10 years old or younger, 8 years old or younger, 6 years old or younger, 4 years old or younger, 2 years old or younger, 1 year old or younger. In particular embodiments, the patient is an adult that is over eighteen years old.

In particular embodiments, the methods reduce or prevent seizures, or symptoms thereof in a patient in need thereof. In particular embodiments, the methods provided may reduce or prevent one or more different types of seizures. Ideally, the methods of the disclosure result in a total prevention of seizures. However, the disclosure also encompasses methods in which the instances of seizures are decreased by at least 10%, at least 20%, at least 30%, at least 40%, at least 50%, at least 60%, at least 70%, at least 80% or at least 90%.

Generally, a seizure can include convulsions, repetitive movements, unusual sensations, and combinations thereof. Seizures can be categorized as focal seizures (also referred to as partial seizures) and generalized seizures. Focal seizures affect only one side of the brain, while generalized seizures affect both sides of the brain. Specific types of focal seizures include simple focal seizures, complex focal seizures, and secondarily generalized seizures. Simple focal seizures can be restricted or focused on a particular lobe (e.g., temporal lobe, frontal lobe, parietal lobe, or occipital lobe). Complex focal seizures generally affect a larger part of one hemisphere than simple focal seizures, but commonly originate in the temporal lobe or the frontal lobe. When a focal seizure spreads from one side (hemisphere) to both sides of the brain, the seizure is referred to as a secondarily generalized seizure. Specific types of generalized seizures include absences (also referred to as petit mal seizures), tonic seizures, atonic seizures, myoclonic seizures, tonic clonic seizures (also referred to as grand mal seizures), and clonic seizures.

In particular embodiments, methods described herein may reduce the frequency of seizures, reduce the severity of seizures, change the type of seizures (e.g., from a more severe type to a less severe type), or a combination thereof in a patient after treatment compared to the absence of treatment (e.g., before treatment), or compared to treatment with an alternative conventional treatment.

Administration of compositions can be by any appropriate route. For example, in particular embodiments, administration may include administration to a cell or tissue slice for research purposes related to Nav1.1 sodium channel dysfunction.

In particular embodiments, administration is to a subject and can be intravenous, retro-orbital, intraocular, intravitreal, parenteral, subcutaneous, intracerebro-ventricular, intramuscular, intrathecal, intraspinal, oral, intraperitoneal, oral, nasal, or direct to a targeted site administration. Delivery can be accomplished by a needle or a cannula or by any other technique of expelling fluidic materials. The methods of administration may also include those modalities as described in U.S. Pat. Nos. 5,543,158; 5,641,515 and 5,399,363.

As is well known in the medical arts, dosages for any one subject depends upon many factors, including the subject's size, surface area, age, the particular compound to be administered, sex, time and route of administration, general health, and other drugs being administered concurrently. Dosages for the compounds of the disclosure will vary, but, in particular embodiments, a dose could be from 105 to 1010 copies of an expression construct of the disclosure. In particular embodiments, a patient receiving intravenous, intraspinal, retro-orbital, intracerebroventricular, or intrathecal administration can be infused with from 106 to 1022 copies of the expression construct.

Therapeutically effective amounts include those that provide effective amounts and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to a disorder associated with Nav1.1 sodium channel dysfunction.

A “therapeutic treatment” includes a treatment administered to a subject who displays symptoms or signs of a disorder associated with Nav1.1 sodium channel dysfunction and is administered to the subject for the purpose of diminishing or eliminating those signs or symptoms of the disorder. The therapeutic treatment can reduce, control, or eliminate the presence or activity of the disorder, the cause of the disorder, and/or reduce control or eliminate side effects of the disorder.

In particular embodiments, the administration of therapeutically-effective amounts of the disclosed compositions may be achieved by a single administration, such as for example, a single injection of sufficient numbers of expression constructs to provide therapeutic benefit to the subject receiving the administration. Alternatively, in some circumstances, it may be desirable to provide multiple, or successive administrations of the compositions, either over a relatively short, or a relatively prolonged period of time.

For example, the number of expression constructs administered to a subject may be 10⁷, 10⁸, 10⁹, 10¹⁰, 10¹¹, 10¹², 10¹³, or even higher, expression constructs/ml given either as a single dose, or divided into two or more administrations as may be required to achieve a desired physiological outcome. In particular embodiments, it may be desirable to administer two or more different expression constructs, either alone, or in combination with one or more other therapeutic drugs to achieve the desired effects of a particular therapy regimen.

Particular dosing and timing of administration for a particular subject can be chosen by a treating physician, researcher, or veterinarian. In other words, the amount of compositions and/or expression constructs and time of administration will be within the purview of the skilled artisan having benefit of the present teachings.

In particular embodiments, treatments for Nav1.1 sodium channel disorders can be combined with another treatment. For example, common conventional therapies for seizures and epilepsy include antiepileptic drugs and non-antiepileptic drug treatments such as low carbohydrate diet (e.g., ketogenic diets, such as classical diet, medium chain triglyceride (MCT) diet, modified Atkins diet (MAD), and low glycemic index treatment (LGIT)), intravenous immunoglobulin, steroids, elimination diet, vagus nerve stimulation, corticetomy, and multiple subpial transections.

Common antiepileptic and anticonvulsive active compounds that may be used in combination with compositions described herein include acetazolamide, cannabidiol, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, and zonisamide.

Kits and Commercial Packages. Kits and commercial packages contain an expression construct described herein. The expression product can be isolated. In particular embodiments, the components of an expression product can be isolated from each other. In particular embodiments, the expression product can be within a vector, within a viral vector, within a cell, within a tissues slice or sample, and/or within a transgenic animal. In particular embodiments, an animal is transgenic following administration of a composition including the expression construct. In particular embodiments, a transgenic animal includes a genetic modification that renders the animal appropriate for use in an animal model of DS. For example, the transgenic animal such as a mouse can be Scn1a^{+/-}. Detailed methods for producing transgenic animals are described in U.S. Pat. No. 4,736,866. Transgenic animals may be of any nonhuman mammalian or avian species, but preferably include mice or nonhuman primates (NHPs). Sheep, horses, cattle, pigs, goats, dogs, cats, rabbits, chickens, and other rodents such as guinea pigs, hamsters, gerbils, rats, and ferrets are also included.

Embodiments of a kit or commercial package will also contain instructions regarding use of the included components, for example, in the research and/or treatment of disorders associated with Nav1.1 sodium channel dysfunction, such as epilepsy and/or DS. Such kits may further include one or more reagents, restriction enzymes, peptides, therapeutics, pharmaceutical compounds, or means for delivery of the compositions such as syringes, injectables, and the like.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. An expression construct (e.g, isolated polynucleotide) including (i) a concatemerized enhancer of SEQ ID: NO: 2 or SEQ ID NO: 6, (ii) a promoter; and (iii) a coding sequence encoding a protein or nucleic acid that rescues voltage-gated sodium channel function in a cell or subject in need thereof.

2. An expression construct of embodiment 1, wherein the concatemer includes a 3× concatemer of SEQ ID: NO: 2 or SEQ ID NO: 6.

3. An expression construct of embodiment 1, wherein the concatemer includes SEQ ID: NO: 2 and SEQ ID NO: 6 arranged in tandem.

4. An expression construct of embodiment 3, wherein the concatemer includes SEQ ID NO: 2-SEQ ID NO: 6-SEQ ID NO: 2; SEQ ID NO: 6-SEQ ID NO: 2-SEQ ID NO: 6; SEQ ID NO: 6-SEQ ID NO: 6-SEQ ID NO: 2; SEQ ID NO: 2-SEQ ID NO: 2-SEQ ID NO: 6; SEQ ID NO: 6-SEQ ID NO: 2-SEQ ID NO: 2; or SEQ ID NO: 2-SEQ ID NO: 6-SEQ ID NO: 6.

5. An expression construct (e.g, isolated polynucleotide) including (i) SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8, (ii) a promoter; and (iii) a coding sequence encoding a protein or nucleic acid that rescues voltage-gated sodium channel function in a cell or subject in need thereof.

6. An expression construct of any of embodiments 1-5, wherein the coding sequence includes or encodes NavSheP-D60N, NavBp, NavMs, 3×HA-NavSheP-D60N, 3×HA-NavBp, 3×HA-NavMs, or His-NavMs.

7. An expression construct of any of embodiments 1-6, wherein the coding sequence includes or encodes human SCN1A, mouse Scn1a, human SCN1A-3×HA, and/or mouse Scn1a-3×ha.

8. An expression construct of any of embodiments 1-7, wherein the coding sequence includes or encodes an artificial transcription factor that increases expression of endogenous SCN1A wherein the artificial transcription factor includes a targeted DNA-binding domain linked to a transcription-activating domain.

9. An expression construct of any of embodiments 1-8, wherein the coding sequence includes or encodes antisense RNA molecules that increase splicing or prevent microRNA negative regulation.

10. An expression construct of any of embodiments 1-9, wherein the coding sequence includes or encodes a nucleotide sequence that upregulates SCN1A expression (e.g., SEQ ID NOs: 66-163).

11. An expression construct of any of embodiments 1-10, wherein the coding sequence includes or encodes and/or one or more segments of SCN1A that assemble into full length SCN1A after expression.

12. An expression construct of embodiment 11 including the SCN1A coding sequence of SEQ ID NO: 58, SEQ ID NO: 59, SEQ ID NO: 60, and SEQ ID NO: 61.

13. An expression construct of embodiment 11 wherein the SCN1A segments include SEQ ID NO: 62, SEQ ID NO: 63, SEQ ID NO: 64, and SEQ ID NO: 65.

14. An expression construct any of embodiments 1-13, wherein the promoter includes minBglobin or minCMV.

15. An expression construct of any of embodiments 1-14, wherein the expression construct is within an adeno-associated viral (AAV) vector.

16. An expression construct of any of embodiments 1-15, wherein the expression construct includes a coding sequence for a reporter protein.

17. An expression construct of embodiment 16, wherein the reporter protein includes a fluorescent reporter protein.

18. An expression construct of any of embodiments 1-17, wherein the expression construct includes or encodes a skipping element.

19. An expression construct of embodiment 18, wherein the skipping element includes a 2A peptide or an internal ribosome entry site (IRES).

20. An expression construct of embodiment 19, wherein the 2A peptide includes T2A, P2A, E2A, and/or F2A.

21. An expression construct of any of embodiments 1-20, wherein the construct includes the elements of CN1367, CN1244, CN1389, CN1390, CN1180, CN1203, CN1498, CN1499, CN1500, CN2001, CN2002, CN2003, CN1504 and CN1512, CN2004 and CN2005, CN2006 and CN2007, CN2008 and CN2009, or CN2026, CN2027, CN2028 and CN2029.

22. An expression construct of any of embodiments 1-21, wherein the construct includes SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and/or SEQ ID NO: 52.

23. An expression construct of any of embodiments 1-22, wherein the construct includes SEQ ID NO: 45 and SEQ ID NO: 46, SEQ ID NO: 47 and SEQ ID NO: 48, SEQ ID NO: 49 and SEQ ID NO: 50, SEQ ID NO: 51, and SEQ ID NO: 52 or CN2026, CN2027, CN2028 and CN2029.

24. An expression construct of any of embodiments 15-23, wherein the AAV vector is associated with a capsid that crosses the blood brain barrier.

25. An expression construct of embodiment 24, wherein the capsid includes PHP.Eb.

26. An expression construct of embodiment 24 or 25, wherein the capsid includes SEQ ID NO: 53.

27. An expression construct of any of embodiments 24-26, wherein the capsid includes an AAV9 capsid with a SEQ ID NO: 54 or SEQ ID NO: 55 insert.

28. An expression construct of any of embodiments 24-27, wherein the capsid includes an AAV2 capsid with a SEQ ID NO: 56 insert.

29. A composition including the expression construct of any of embodiments 1-28.

30. A cell including the expression construct of any of embodiments 1-28.

31. A non-human animal including the expression construct of any of embodiments 1-28.

32. A kit including the expression construct of any of embodiments 1-28.

33. A method of rescuing voltage-gated sodium channel function in a defective cell in need thereof including administering a therapeutically effective amount of a composition of embodiment 29 to the cell.

34. A method of rescuing voltage-gated sodium channel function in a subject in need thereof including administering a therapeutically effective amount of a composition of embodiment 29 to the subject.

35. A method of embodiment 34, wherein the subject is need thereof due to a diagnosis of epilepsy, an SCN1A-related seizure disorder, simple febrile seizures (FS), generalized epilepsy with febrile seizures plus (GEFS+), Dravet Syndrome (DS), intractable childhood epilepsy with generalized tonic-clonic seizures (ICE-GTC), intractable infantile partial seizures, myoclonic-astatic epilepsy, Lennox-Gastaut syndrome (LGS), or infantile spasms.

36. A method of embodiment 34 or 35, wherein the subject is a pediatric patient.

37. A method of any of embodiments 34-36, wherein the subject is less than 4 years old.

38. A method of embodiment 34, 35, or 37, wherein the subject is a transgenic Scn1a^{+/-} mouse undergoing a temperature-induced febrile seizure test.

39. A method of any of embodiments 34-38, wherein the composition is administered intravenously.

40. A method of any of embodiments 34-39, wherein the composition is administered intrathecally into cerebrospinal fluid, via the lateral ventricles or cisterna magna or lumbar space or cannula into the foramen magnum.

41. An expression construct (e.g, isolated polynucleotide) including:
(a) a non-naturally occurring enhancer sequence; and
(b) a nucleic acid encoding a protein or nucleotide sequence that rescues voltage-gated sodium channel function;
   wherein the enhancer sequence:
(i) consists of the sequence of SEQ ID NO: 3;
(ii) includes two or more copies of SEQ ID NO: 2 or SEQ ID NO: 6 arranged in tandem (e.g, SEQ ID NO: 2-SEQ ID NO: 6-SEQ ID NO: 2; SEQ ID NO: 6-SEQ ID NO: 2-SEQ ID NO: 6; SEQ ID NO: 6-SEQ ID NO: 6-SEQ ID NO: 2; SEQ ID NO: 2-SEQ ID NO: 2 SEQ ID NO: 6; SEQ ID NO: 6-SEQ ID NO: 2-SEQ ID NO: 2; or SEQ ID NO: 2-SEQ ID NO: 6-SEQ ID NO: 6; SEQ ID NO: 6-SEQ ID NO: 6-SEQ ID NO: 6);
(iii) includes a sequence having at least 90% sequence identity with SEQ ID NO: 3 and maintaining interneuron-specific enhancer function; or
(iv) a sequence the complementary strand of which is capable of hybridizing to the sequence of (i), (ii), or (iii); and
wherein the enhancer promotes the transcription of the nucleic acid selectively within inhibitory neurons following administration to a sample or subject.

42. An expression construct of embodiment 41, wherein nucleic acid includes or encodes NavSheP-D60N, NavBp, NavMs, 3×HA-NavSheP-D60N, 3×HA-NavBp, 3×HA-NavMs, or His-NavMs.

43. An expression construct of embodiment 41 or 42, wherein the nucleic acid includes or encodes human SCN1A, mouse Scn1a, human SCN1A-3×HA, and/or mouse Scn1a-3×ha.

44. An expression construct of any of embodiments 41-43, wherein the nucleic acid includes or encodes an artificial transcription factor that increases expression of endogenous SCN1A wherein the artificial transcription factor includes a targeted DNA-binding domain linked to a transcription-activating domain.

45. An expression construct of any of embodiments 41-44, wherein the nucleic acid includes or encodes antisense RNA molecules that increase splicing or prevent microRNA negative regulation.

46. An expression construct of any of embodiments 41-45, wherein the nucleic acid includes or encodes a nucleotide sequence that upregulates SCN1A expression (e.g., SEQ ID NOs: 66-163).

47. An expression construct of any of embodiments 41-46, wherein the nucleic acid includes or encodes and/or one or more segments of SCN1A that assemble into full length SCN1A after expression.

48. An expression construct of embodiment 47 including nucleic acid including the SCN1A coding sequence of SEQ ID NO: 58, SEQ ID NO: 59, SEQ ID NO: 60, and SEQ ID NO: 61.

49. An expression construct of embodiment 47 wherein the SCN1A segments include SEQ ID NO: 62, SEQ ID NO: 63, SEQ ID NO: 64, and SEQ ID NO: 65.

50. An expression construct of any of embodiment 41-49, including a promoter including minBglobin or minCMV.

51. An expression construct of any of embodiments 41-50, wherein the expression construct is within an adeno-associated viral (AAV) vector.

52. An expression construct of any of embodiments 41-51, wherein the expression construct includes a nucleic acid encoding a reporter protein.

53. An expression construct of embodiment 52, wherein the reporter protein includes a fluorescent reporter protein.

54. An expression construct of any of embodiments 41-53, wherein the expression construct includes or encodes a skipping element.

55. An expression construct of embodiment 54, wherein the skipping element includes a 2A peptide or an internal ribosome entry site (IRES).

56. An expression construct of embodiment 55, wherein the 2A peptide includes T2A, P2A, E2A, and/or F2A.

57. An expression construct of any of embodiments 41-56, wherein the construct includes the elements of CN1367, CN1244, CN1389, CN1390, CN1180, CN1203, CN1498, CN1499, CN1500, CN2001, CN2002, CN2003, CN1504 and CN1512, CN2004 and CN2005, CN2006 and CN2007, or CN2008 and CN2009.

58. An expression construct of any of embodiments 41-57, wherein the construct includes SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and/or SEQ ID NO: 52.

59. An expression construct of any of embodiments 41-58, wherein the construct includes SEQ ID NO: 45 and SEQ ID NO: 46, SEQ ID NO: 47 and SEQ ID NO: 48, SEQ ID NO: 49 and SEQ ID NO: 50, or SEQ ID NO: 51, and SEQ ID NO: 52

60. An expression construct of any of embodiments 51-59, wherein the AAV vector is associated with a capsid that crosses the blood brain barrier.

61. An expression construct of embodiment 60, wherein the capsid includes PHP.Eb.

62. An expression construct of embodiment 60 or 61, wherein the capsid includes SEQ ID NO: 53.

63. An expression construct of any of embodiments 60-62, wherein the capsid includes AAV9 capsid with a SEQ ID NO: 54 or SEQ ID NO: 55 insert.

64. An expression construct of any of embodiments 60-63, wherein the capsid includes AAV2 capsid with a SEQ ID NO: 56 insert.

65. A composition including the expression construct of any of embodiments 41-64.

66. A cell including the expression construct of any of embodiments 41-64.

67. A non-human animal including the expression construct of any of embodiments 41-64.

68. A kit including the expression construct of any of embodiments 41-64.

69. A method of rescuing voltage-gated sodium channel function in a defective cell in need thereof including administering a therapeutically effective amount of a composition of embodiment 65 to the cell.

70. A method of rescuing voltage-gated sodium channel function in a subject in need thereof including administering a therapeutically effective amount of a composition of embodiment 65 to the subject.

71. A method of embodiment 70, wherein the subject is need thereof due to a diagnosis of epilepsy, an SCN1A-related seizure disorder, simple febrile seizures (FS), generalized epilepsy with febrile seizures plus (GEFS+), Dravet Syndrome (DS), intractable childhood epilepsy with generalized tonic-clonic seizures (ICE-GTC), intractable infantile partial seizures, myoclonic-astatic epilepsy, Lennox-Gastaut syndrome (LGS), or infantile spasms.

72. A method of embodiment 70 or 71, wherein the subject is a pediatric patient.

73. A method of any of any of embodiments 70-72, wherein the subject is less than 4 years old.

74. A method of any of embodiments 70, 71, or 73, wherein the subject is a transgenic Scn1a^{+/-} mouse undergoing a temperature-induced febrile seizure test.

75. A method of any of embodiments 70-74, wherein the composition is administered intravenously.

76. A method of any of embodiments 70-75, wherein the composition is administered intrathecally into cerebrospinal fluid, via the lateral ventricles or cisterna magna or lumbar space or cannula into the foramen magnum.

77. A vector system including two AAV vectors wherein the first vector encodes the N-terminal portion of a protein that rescues voltage-gated sodium channel activity in a cell in need thereof, and the second vector encodes the C-terminal portion of the protein, wherein the portion of the gene encoded by the two vectors overlaps to provide a region of homology for homologous recombination to produce the full-length Nav1.1 protein, and wherein the first vector includes a promoter and an enhancer that consists of SEQ ID NO: 3 but does not include a termination signal or a polyA signal and the second vector includes a termination signal and a polyA signal but does not include a promoter or an enhancer.

78. A vector system of embodiment 77, wherein the protein includes human SCN1A.

79. A vector system of embodiment 77 or 78, wherein the region of homology is 75-1000 base pairs.

80. A vector system of embodiment 77 or 78, wherein the region of homology is 550-650 base pairs.

81. A vector system of any of embodiments 77-80, wherein the vectors selectively express the protein in inhibitory neurons.

82. A vector system of any of embodiments 77-81, wherein the region of homology further includes an intron element with a splice donor site on the first vector and a splice acceptor site on the second vector, so that trans-splicing across the homologous region drives more efficient full-length protein reconstitution following recombination.

83. A vector system of any of embodiments 77-82, wherein the vector system includes the elements of CN1504, CN1512, CN2004, CN2005, CN2006, CN2007, CN2008, and/or CN2009.

84. A vector system of any of embodiments 77-83, wherein the vector system includes the elements of CN1504 and CN1512, CN2004 and CN2005, CN2006 and CN2007, or CN2008 and CN2009.

85. A vector system of any of embodiments 77-84, wherein the vector system includes SEQ ID NO: 45 and SEQ ID NO: 46, SEQ ID NO: 47 and SEQ ID NO: 48, SEQ ID NO: 49 and SEQ ID NO: 50, or SEQ ID NO: 51, and SEQ ID NO: 52.

86. A viral vector including SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, or SEQ ID NO: 52.

87. An expression construct including SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8 and a coding sequence encoding an artificial transcription factor including a specific DNA-binding domain linked to a general transcription-activating domain wherein the artificial transcription factor increases the expression of the endogenous functional copy of SCN1A (or other SCN_A family gene).

88. An expression construct including SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8 and a coding sequence encoding an artificial transcription factor including a specific DNA-binding domain linked to a general transcription-activating domain wherein the artificial transcription factor increases the expression of the endogenous functional copy of SCN1A (or other SCN_A family gene).

89. An expression construct including SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8 and a coding sequence encoding antisense RNA molecules that increase splicing and/or prevent microRNA negative regulation of SCN1A expression.

90. A set of expression constructs including SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8 wherein the set of expression constructs delivers hSCN1A protein as two or four subunit ORFs delivered by two or four separate vectors.

91. An isolated polynucleotide including an enhancer including the sequence of SEQ ID NO: 3, and further including a heterologous inducible promoter and a gene encoding a protein or nucleic acid sequence that rescues Nav1.1 channel function selectively in inhibitory neurons, wherein the enhancer and the heterologous inducible promoter are operably linked to the gene.

92. A vector including the isolated polynucleotide of embodiment 91.

93. A vector of embodiment 92, wherein the vector is a viral vector.

94. A vector of embodiment 93, wherein the viral vector is an AAV viral vector. 94.

95. A human or non-human cell including the isolated polynucleotide of embodiment 91.

96. A set of vectors for selectively driving expression of a protein or nucleic acid sequence that rescues Nav1.1 channel function in inhibitory neurons including the vector of any of embodiment 92-94, and a second vector including an enhancer selected from SEQ ID NOS: 1, 2, 4, 5, 6, 7 or 8.

97. A method for selectively driving expression of a protein or nucleic acid sequence that rescues Nav1.1 channel function in inhibitory neurons including (1) providing the vector of any of embodiments 92-94, (2) using the vector to generate a transgenic mouse, and (3) detecting rescued Nav1.1 channel function in the transgenic mouse.

98. A method of embodiment 97, wherein the rescued Nav1.1 channel function is within inhibitory neurons.

99. A set of coding sequences including the coding sequences of the hSCN1A_Fragments within SEQ ID NO: 58, SEQ ID NO: 59, SEQ ID NO: 60, and SEQ ID NO: 61.

100. A set of vectors including CN2026, CN2027, CN2028, and CN2029.

Example 1. In relation to FIG. 1A, living human temporal cortex brain tissue was excised during neurosurgery to remove an epileptic focus. This tissue was dissected and sliced to 350-micron thick tissue slices, which were cultured on semipermeable membranes with semisynthetic culture medium as described by Ting et al. (Scientific Reports 8(1): 8407, 2018). On the first day of culture, slices were infected with purified virus CN1180/DJ (which labels inhibitory neurons) by applying purified virus directly to the brain slice. At 7 days post infection, the electrophysiology of SYFP2⁺ (visible in the green channel) infected cells was assessed by patch-clamp recordings. The recorded cells were backfilled with Alexa 594 dye (visible in the red channel) to perform post-hoc visualization of their cell bodies and morphologies.

In relation to FIG. 1B, patch-clamp recordings were performed on multiple SYFP2⁺ and SYFP2⁻ cells. Their backfilled morphologies were tabulated as being pyramidal (indicative of excitatory neurons) or non-pyramidal (indicative of inhibitory neurons). This analysis indicated that SYFP2⁺ cells are largely of inhibitory neuron character.

In relation to FIG. 1C, the electrophysiological character of multiple SYFP2⁺ and SYFP2 cells was analyzed. These analyses indicated that, compared to SYFP2 cells, the SYFP2⁺ cells had shorter action potential (AP) half-widths, had greater firing rates, had faster rates of depolarization, and had faster rates of AP firing in response to injected current (FI slope). These metrics all suggest that SYFP2⁺ cells are fast-spiking interneurons.

In relation to FIG. 1D, after performing patch-clamp electrophysiology tissue was fixed and immunostained with anti-GFP and anti-Parvalbumin antibodies. This analysis indicated that many GFP⁺ cells are Parvalbumin⁺, which suggests that these cells have molecular identities consistent with fast-spiking interneurons.

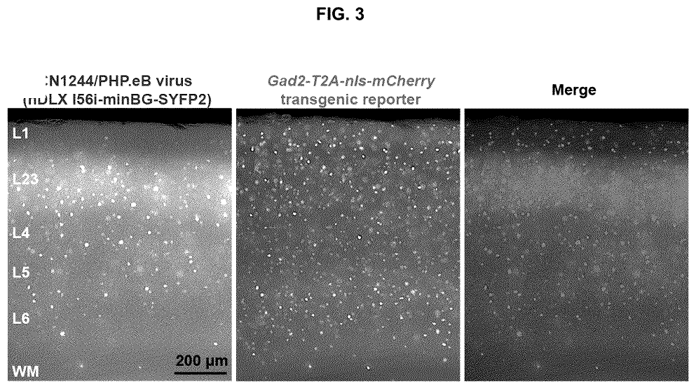

In relation to FIG. 3, a Gad2-T2A-nls-mCherry (Peron et al., Neuron 86:783-799, 2015) mouse was injected retro-orbitally with 1E11 genome copies of the virus CN1244/PHP.eB. After 3 weeks the brain was harvested, sliced into 350-micron thick slices, and then imaged for SYFP2 and mCherry expression to visualize virus-expressing cells and all interneurons, respectively.

In relation to FIGS. 4A, 4B, adult wild type mice were retro-orbitally injected with 1E+11 genome copies of the indicated viruses. Animals were maintained for 3-4 weeks, then euthanized, with brains extracted and sliced, followed by live tissue epifluorescence imaging of native fluorescence. Exposure times were matched to allow direct comparison of transgene expression levels. The first three panels are a 500 msec exposure for each of the indicated constructs; the fourth panel is a shorter (50 msec) exposure image of CN1390.

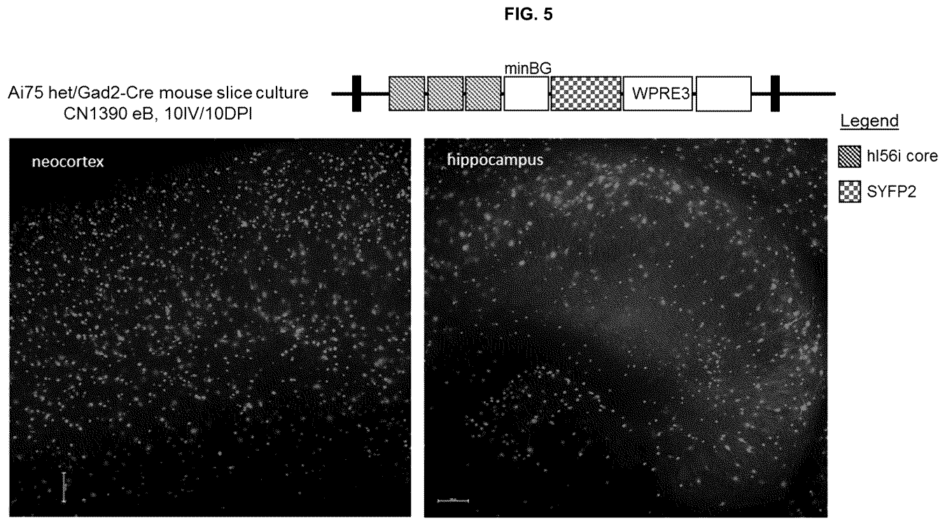

In relation to FIG. 5, cortical/hippocampal brain slice cultures were prepared from P5-10 Gad2-IRES-Cre heterozygous; Ai75 heterozygous animals. These animals have Cre-mediated activation of a nuclear-tagged tdTomato transgene in Gad2-expressing cells, resulting in bright nuclear red fluorescence in inhibitory neurons. One hour after culturing, CN1390 viral suspension was pipetted onto the slice surface to transduce brain cell types. At 10 DIV/10 DPI, native fluorescence was imaged in green and red channels on a Nikon inverted microscope.

In relation to FIGS. 6A-6E, human ex vivo neocortical brain slice cultures were prepared from live neurosurgical specimens (Ting et al., Scientific Reports 8(1): 8407, 2018). One hour after culturing, CN1390 viral suspension was pipetted onto the slice surface to transduce brain cell types. At 1, 3, and 6 DIV/DPI, native SYFP2 fluorescence was imaged using matched exposure times on aa Nikon microscope. FIGS. 6A-6D illustrate rapid viral-genetic labeling of human neocortical interneurons for targeted patch clamp recording and analysis. At various times in culture, slices were taken for terminal patch clamp recording analysis to establish the firing properties of labeled neurons. Functional analysis of human neocortical interneuron firing patterns and electrical properties by patch clamp recording was feasible as early as 40 hours post-infection with CN1390 eB virus.

Referring to FIG. 9, the top vector includes the enhancer of SEQ ID NO: 3 and promoter (minCMV) elements to drive expression in all inhibitory neurons (including both Pvalb+ and Pvalb-inhibitory neurons). The driven transgene includes a SYFP2 linked by a P2A tag to the N-terminal region of human SCN1A, which includes 604 bp homology to the C-terminal region included on the bottom vector. The bottom vector also includes a C-terminal 3×HA tag and 3′UTR regulatory sequences (WPRE3 and polyA sites).

Both AAV viral vectors were packaged into PHP.eB capsid and delivered both intravenously to a single C57BI/6 mouse. After 21 days the mouse brain was harvested, fixed, and processed for immunofluorescence with anti-GFP (targeting SYFP2), anti-HA, and anti-Pvalb antibodies to detect transgene-expressing cells and their overlap with Pvalb₊ inhibitory neurons (which are cells having particular importance in DS symptomology). Many inhibitory neurons were labeled with the N-terminal SYFP2₊ tag at high level, including both Pvalb₊ and Pvalb₋ inhibitory neurons. Some of these SYFP2₊ cells also express HA which indicates fully intact SCN1A protein product being expressed in these GFP₊HA₊ cells (arrows). The vertical arrows indicate Pvalb₊ interneurons expressing human SCN1A and the horizontal arrows indicate Pvalb-interneurons expressing human SCN1A. These SCN1A-expressing cells are found throughout the forebrain (both neocortex and hippocampus [here dentate gyrus]), which includes the brain region known to be important for most epilepsies and known to be dysfunctional in DS.

Example 2. Dravet syndrome (DS) is a drug-resistant and life-threatening form of epilepsy. It typically begins in the first year of life, with fever- or temperature-induced seizures that evolve into generalized clonic, tonic-clonic, and unilateral seizures. These seizures are often resistant to current anti-epileptic drugs, the first-line therapies for this syndrome; complete seizure control is typically not achieved. As the disease progresses, most affected children also suffer from comorbid conditions including developmental delays, intellectual disabilities, impaired motor control and coordination, autistic behaviors, sleep disturbances, and many die prematurely.

Heterozygous loss-of-function mutations in SCN1A, the gene that encodes the pore-forming subunit of the voltage-gated sodium channel Nav1.1 are the most common cause of DS and occur in nearly 1/16,000 newborns.

A mouse model, generated by knock-out of Scn1a, replicates the several key phenotypic features of this epilepsy including infantile (P21)-epilepsy onset, high susceptibility to thermal seizures, ataxia, spontaneous seizures, sleep impairments, autistic behaviors, and premature death. Seizures and several comorbidities arise from impaired interneuron function in these mice.

This mouse model was used to investigate the efficacy of a new viral vector for DS. The virus was delivered by retro-orbital injection using an insulin syringe and its ability to suppress seizure was evaluated using the thermal seizure test. In this test, the mouse body core temperature is elevated slowly, using a temperature controller and a heat lamp, until a seizure occurs, or 42.5° C. is attained. The temperature of seizure onset in treated and control mice are compared to determine the efficacy of the intervention. In additional tests, the efficacy of treatment on spontaneous seizure and premature mortality are assessed using video and electroencephalographic monitoring.

The viral vector is a new AAV viral vector named CN1500. This viral vector is a recombinant AAV that expresses the transgene SYFP2-P2A-NavSheP-D60N to rescue the loss of the voltage-gated sodium channel Nav1.1. NavSheP-D60N is a modified voltage-gated sodium channel of bacterial origin that has been modified to improve the kinetics and expression in mammalian cells. The transgene expression level is elevated by the addition of a WPRE3 element, and transcription is terminated with the bovine growth hormone poly adenylation sequence. Expression of the transgene is high and limited to inhibitory cells in forebrain structures including the cortex and the hippocampus, via the 3×hi56iCore synthetic enhancer (SEQ ID NO: 3) directly 5′ of a CMV minimal promoter. Furthermore, the therapeutic transgene NavSheP-D60N is labeled by an HA epitope tag to verify correct protein localization.

To test the efficacy of the therapeutic AAV viral vector, CN1500 package using the PHP.eB serotype was used. A cohort of postnatal day 35 Scn1a^{+/-} mice were either injected with 2×10¹¹ vg per animal or were left un-injected. The AAV was introduced intravenously using the retro-orbital delivery route. Two weeks after viral administration, animals from the treatment and control groups were assessed for their susceptibility to febrile seizures. As indicated previously, febrile seizures were measured by steadily raising the mouse's temperature under a heat lamp 0.5 Celsius every two minutes and measuring the internal temperature of the mouse with a rectal probe. The temperature where the mouse experienced a seizure is recorded.

The new therapeutic vector CN1500 was both highly expressed in mouse cortical and hippocampal GABAergic cells, but also raised the average temperature where Son1a^{+/-} mice experienced febrile seizures from 38.7° C. to 41° C. These data show that CN1500 can substantially rescue the loss of Scn1a.

Example 2 references include: Catterall et al. (2010) The Journal of physiology 588:1849-1859; Cheah et al. (2012) Proceedings of the National Academy of Sciences of the United States of America 109:14646-14651; Kalume (2013) Respir Physiol Neurobiol. 189(2): 324-8; Kalume et al., (2007) J Neurosci 27:11065-11074; Kalume et al., (2013) The Journal of clinical investigation 123:1798-1808; Oakley et al., (2009) Proceedings of the National Academy of Sciences of the United States of America 106:3994-3999.

### Description of Sequences

Nucleic acid sequences described herein are shown using standard letter abbreviations for nucleotide bases, as defined in 37 C.F.R. § 1.822. Only one strand of each nucleic acid sequence is shown, but the complementary strand is understood as included in embodiments where it would be appropriate.

SEQ ID NO: 1: nucleic acid sequence of the human (h) DLX 156i enhancer;

SEQ ID NO: 2: nucleic acid sequence of the hl56i core enhancer;

SEQ ID NO: 3: nucleic acid sequence of the 3×h156iCore enhancer;

SEQ ID NO: 4: nucleic acid sequence of the murine DLX 156i enhancer;

SEQ ID NO: 5: nucleic acid sequence of the zebrafish DLX 156i enhancer;

SEQ ID NO: 6: nucleic acid sequence of the zebrafish 156i core;

SEQ ID NO: 7: nucleic acid sequence of the zebrafish 3×156i core;

SEQ ID NO: 8: hDLX 112b enhancer;

SEQ ID NO: 9: NavSheP-D60N, codon optimized, with N-terminal 3×HA tag; SEQ ID NO: 10: NavSheP endogenous sequence;

SEQ ID NO: 11: NavBp, endogenous sequence;

SEQ ID NO: 12: NavBp, codon optimized, with N-terminal 3×HA tag; SEQ ID NO: 13: NavMs, endogenous sequence;

SEQ ID NO: 14: NavMs, codon optimized, with N-terminal 3×HA tag and linker;

SEQ ID NO: 15: NavMs, codon optimized, with N-terminal His tag and linker;

SEQ ID NO: 16: Human SCN1A; SEQ ID NO: 17: SYFP2;

SEQ ID NO: 18: P2A Encoding Sequence; SEQ ID NO: 19: WPRE3;

SEQ ID NO: 20: BGHpA;

SEQ ID NO: 21: N-terminal 3×HA tag (Protein);

SEQ ID NO: 22: N-terminal 3×HA tag (DNA);

SEQ ID NO: 23: hSCN1A N-term of two-part expression system;

SEQ ID NO: 24: hSCN1A C-term of two-part expression system with c-terminal 3×HA sequence;

SEQ ID NO: 25: 604 bp homology region of hSCN1A N term and C term that can be used in two-part expression system;

SEQ ID NO: 26: P2A Translation from CN1498;

SEQ ID NO: 27: T2A; SEQ ID NO: 28: E2A;

SEQ ID NO: 29: F2A;

SEQ ID NO: 30: MinBglobin; SEQ ID NO: 31: minCMV;

SEQ ID NO: 32: AAV9 PHP.eB capsid replacement sequence;

SEQ ID NO: 33: CN1367-portion between L-ITR and R-ITR: positions 142-2984;

SEQ ID NO: 34: CN1500-portion between L-ITR and R-ITR: positions 142-2976;

SEQ ID NO: 35: CN1498-portion between L-ITR and R-ITR: positions 142-2943;

SEQ ID NO: 36: CN1499-portion between L-ITR and R-ITR: positions 142-2946;

SEQ ID NO: 37: CN1244-portion between L-ITR and R-ITR: positions 142-2042;

SEQ ID NO: 38: CN1389-portion between L-ITR and R-ITR positions 142-1897;

SEQ ID NO: 39: CN1390-portion between L-ITR and R-ITR positions 142-1660;

SEQ ID NO: 40: CN1203-portion between L-ITR and R-ITR positions 183-2052;

SEQ ID NO: 41: CN1180-portion between L-ITR and R-ITR positions 183-1891;

SEQ ID NO: 42: CN2001-portion between L-ITR and R-ITR positions 142-2023;

SEQ ID NO: 43: CN2002-portion between L-ITR and R-ITR positions 142-1993;

SEQ ID NO: 44: CN2003-portion between L-ITR and R-ITR positions 142-2056;

SEQ ID NO: 45: CN1504-portion between L-ITR and R-ITR positions 142-4489;

SEQ ID NO: 46: CN1512-portion between L-ITR and R-ITR positions 142-4165;

SEQ ID NO: 47: CN2004-portion between L-ITR and R-ITR positions 142-3792;

SEQ ID NO: 48: CN2005-portion between L-ITR and R-ITR positions 142-4160;

SEQ ID NO: 49: CN2006-portion between L-ITR and R-ITR positions 142-4790;

SEQ ID NO: 50: CN2007-portion between L-ITR and R-ITR positions 142-4671;

SEQ ID NO: 51: CN2008-portion between L-ITR and R-ITR positions 142-3995;

SEQ ID NO: 52: CN2009-portion between L-ITR and R-ITR positions 142-4525;

SEQ ID NO: 53: epitope isolated during in vivo screening of a random AAV display peptide library;

SEQ ID NO: 54: 7-mer sequence of AAV-PHP.S; SEQ ID NO: 55: 7-mer sequence of AAV-PHP.B;

SEQ ID NO: 56: 7-mer sequence of AAV-PPS;

SEQ ID NO: 57: AAV9 VP1 capsid protein sequence (UniProt Accession number Q6JC40);

SEQ ID NO: 58: CN2026-rAAV-3×hl56i (core)-minBG-hSCN1A_Fragment1-WPRE3-BGHpA;

SEQ ID NO: 59: CN2027-rAAV-3×hl56i (core)-minBG-hSCN1A_Fragment2-WPRE3-BGHpA;

SEQ ID NO: 60: CN2028-rAAV-3×hl56i (core)-minBG-hSCN1A_Fragment3-WPRE3-BGHpA;

SEQ ID NO: 61: CN2029-rAAV-3×hl56i (core)-minBG-hSCN1A_Fragment4-WPRE3-BGHpA;

SEQ ID NO: 62: >hSCN1A_Fragment1_ProteinSequence;

SEQ ID NO: 63: hSCN1A_Fragment2_ProteinSequence; SEQ ID NO: 64: hSCN1A_Fragment3_ProteinSequence;

SEQ ID NO: 65: hSCN1A_Fragment4_ProteinSequence;

SEQ ID NOs. 66-163: Nucleotide sequences that result in upregulation of SCNA1.

Variants of the sequences disclosed and referenced herein are also included. Guidance in determining which amino acid residues can be substituted, inserted, or deleted without abolishing biological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153(1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410(1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE-3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

In particular embodiments references to gene editing systems, such as CRISPR and Cas-9 (also known as Csnl and Csxl2) above should be interpreted to include additional options and developments, as is understood by one of ordinary skill in the art. For example, any gene editing system capable of precise genomic recognition and binding can be used. When nucleases are used for targeted genome binding without associated cutting and editing, the nucleases can lack cutting functionality.

In relation to the use of Cas-9, numerous other options for Cas proteins are available and appropriate for use. Cas-9 itself can refer to one or more catalytic domains of a Cas9 protein derived from bacteria such as Corynebacter, Sutterella, Legionella, Treponema, Filif actor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g. the two catalytic domains are derived from different bacterial species. Additional exemplary Cas nucleases that can be used to bind DNA include CasI, CasIB, Cas2, Cas3, Cas4, Cas5, Cas6, Cas7, Cas8, CasIO, Cpf1, C2c3, C2c2 and C2clCsyl, Csy2, Csy3, CseI, Cse2, CscI, Csc2, Csa5, Csn2, Csm2, Csm3, Csm4, Csm5, Csm6, CmrI, Cmr3, Cmr4, Cmr5, Cmr6, Cpf1, CsbI, Csb2, Csb3, Csx17, Csxl4, CsxIO, CsxI6, CsaX, Csx3, CsxI, CsxI5, CsfI, Csf2, Csf3, and Csf4.

The Cpf1 nuclease particularly can provide added flexibility in target site selection by means of a short, three base pair recognition sequence (TTN), known as the protospacer-adjacent motif or PAM. Particular embodiments can utilize engineered Cpf1s. For example, US 2018/0030425 describes engineered Cpf1 nucleases from Lachnospiraceae bacterium ND2006 and Acidaminococcus sp. BV3L6 with altered and improved target specificity.

Other Cpf1 variants include Cpf1 homologs and orthologs of the Cpf1 polypeptides disclosed in Zetsche et al. (2015) Cell 163:759-771 as well as the Cpf1 polypeptides disclosed in U.S. 2016/0208243. Other engineered Cpf1 variants are known to those of ordinary skill in the art and included within the scope of the current disclosure (see, e.g., WO/2017/184768).

Additional information regarding CRISPR-Cas systems and components thereof are described in, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Zinc finger nucleases (ZFNs) are synthesized by fusing a zinc finger DNA-binding domain to a DNA cleavage domain. Particular embodiments described herein can utilize zinc fingers to bind specific DNA sequences. The DNA-binding domain includes three to six zinc finger proteins which are similar to those found in transcription factors. The DNA cleavage domain includes the catalytic domain of, for example, FokI endonuclease. The FokI domain functions as a dimer requiring two constructs with unique DNA binding domains for sites on either side of the target site cleavage sequence. The FokI cleavage domain cleaves within a five or six base pair spacer sequence separating the two inverted half-sites.

For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140 as well as Gaj et al., Nat Methods, 2012, 9(8): 805-7; Ramirez et al., Nucl Acids Res, 2012, 40(12): 5560-8; Kim et al., Genome Res, 2012, 22(7): 1327-33; Urnov et al., Nature Reviews Genetics, 2010, 11:636-646; Miller, et al. Nature biotechnology 25, 778-785(2007); Bibikova, et al. Science 300, 764(2003); Bibikova, et al. Genetics 161, 1169-1175 (2002); Wolfe, et al. Annual review of biophysics and biomolecular structure 29, 183-212(2000); Kim, et al. Proceedings of the National Academy of Sciences of the United States of America 93, 1156-1160(1996); and Miller, et al. The EMBO journal 4, 1609-1614(1985).

Particular embodiments can use transcription activator like effector nucleases (TALENs) as gene editing agents. TALENs have been engineered to bind a target genetic sequence and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12th and 13th positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant FokI endonucleases. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14 (I): 49-55; Beurdeley et al., Nat Commun, 2013, 4:1762; Scharenberg et al., Curr Gene Ther, 2013, 13(4): 291-303; Gaj et al., Nat Methods, 2012, 9(8): 805-7; Miller, et al. Nature biotechnology 29, 143-148(2011); Christian, et al. Genetics 186, 757-761(2010); Boch, et al. Science 326, 1509-1512(2009); and Moscou, & Bogdanove, Science 326, 1501 (2009).

Particular embodiments can utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Exemplary meganucleases include I-SceI, I-Scell, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsbIVP, I-TIiI, I-PpoI, PI-PspI, F-SceI, F-SceII, F-SuvI, F-TevI, F-TevII, I-AmaI, I-AniI, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LIaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, 1-PobIP, I-PorI, I-PorIIP, I-PbpIP, I-SpBetaIP, I-ScaI, I-SexIP, 1-SneIP, I-SpomI, I-SpomCP, I-SpomIP, I-SpomIIP, I-SquIP, I-Ssp68031, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, 1-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TIiI, and PI-THII.

Particular embodiments described herein can utilize gene editing systems to insert expression constructs within targeted genomic safe harbors. Methods for identifying genomic safe harbor sites are described in Sadelain et al., Nature Reviews (2012); 12:51-58; and Papapetrou et al., Nat Biotechnol. (2011) January; 29(1): 73-8.

In particular embodiments, expression constructs refer to isolated polynucleotide sequences that include no elements, portions, or nucleotides that are not described in relation to the construct.

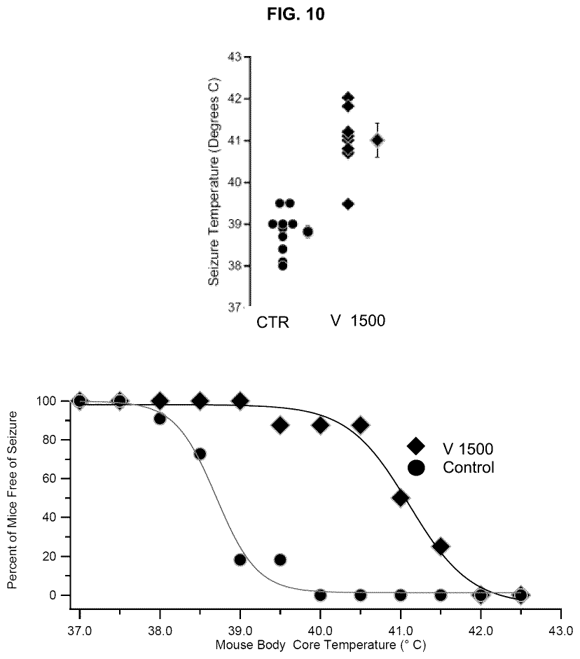

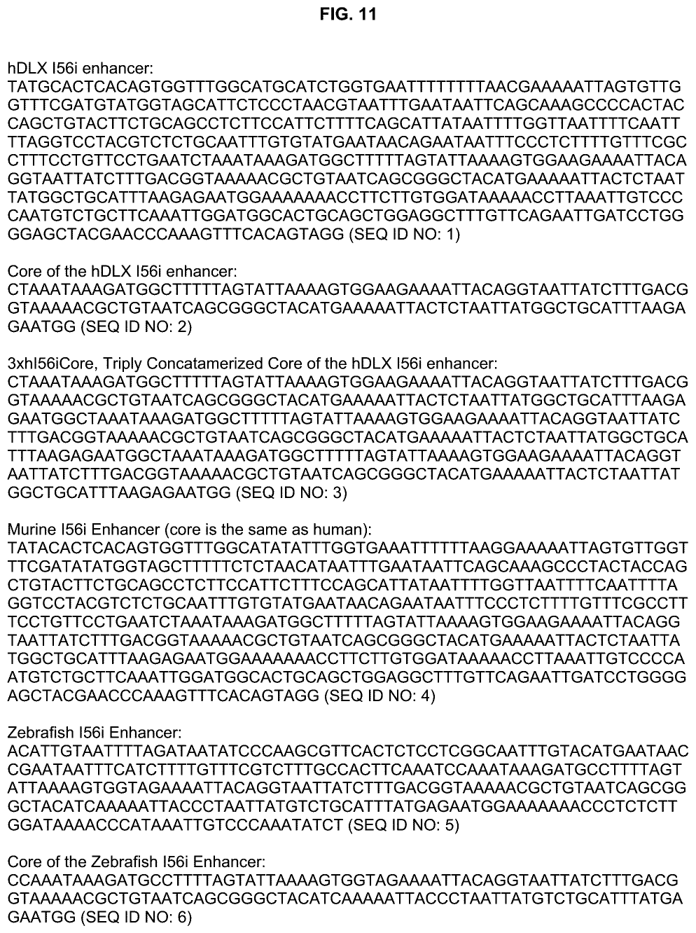

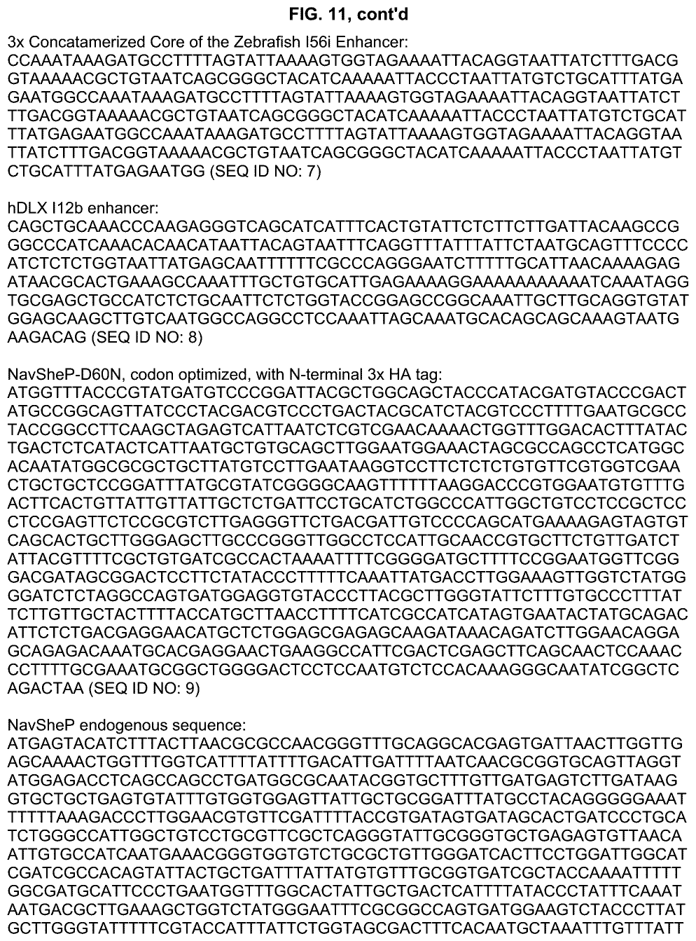

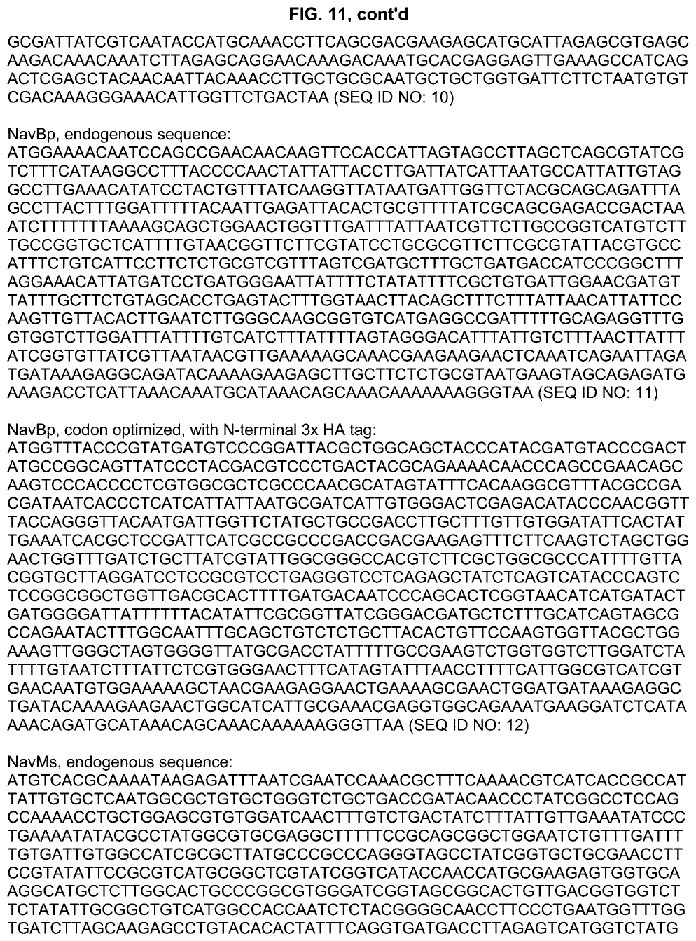

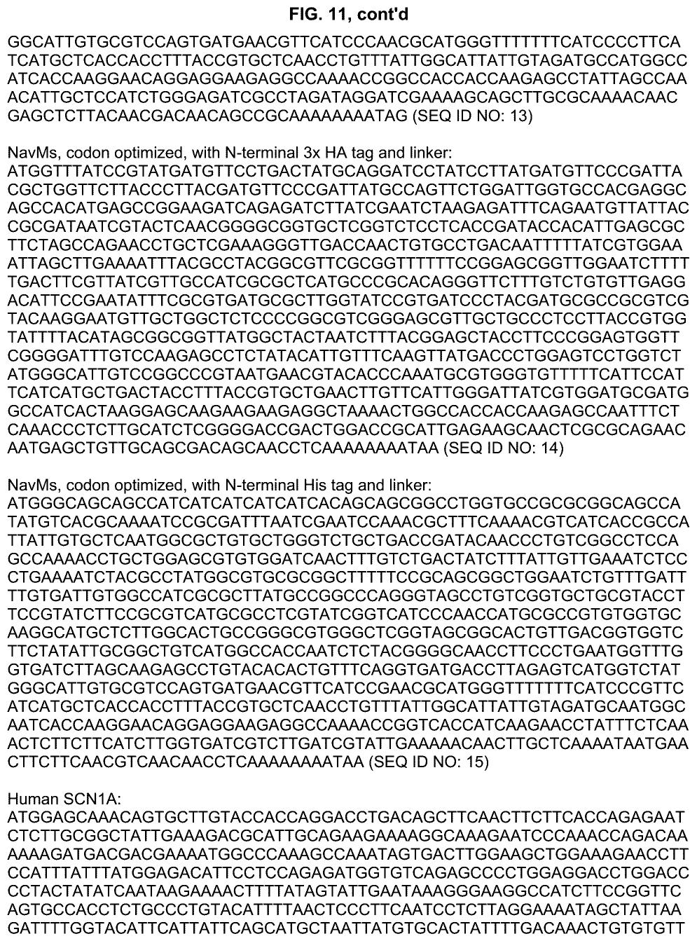

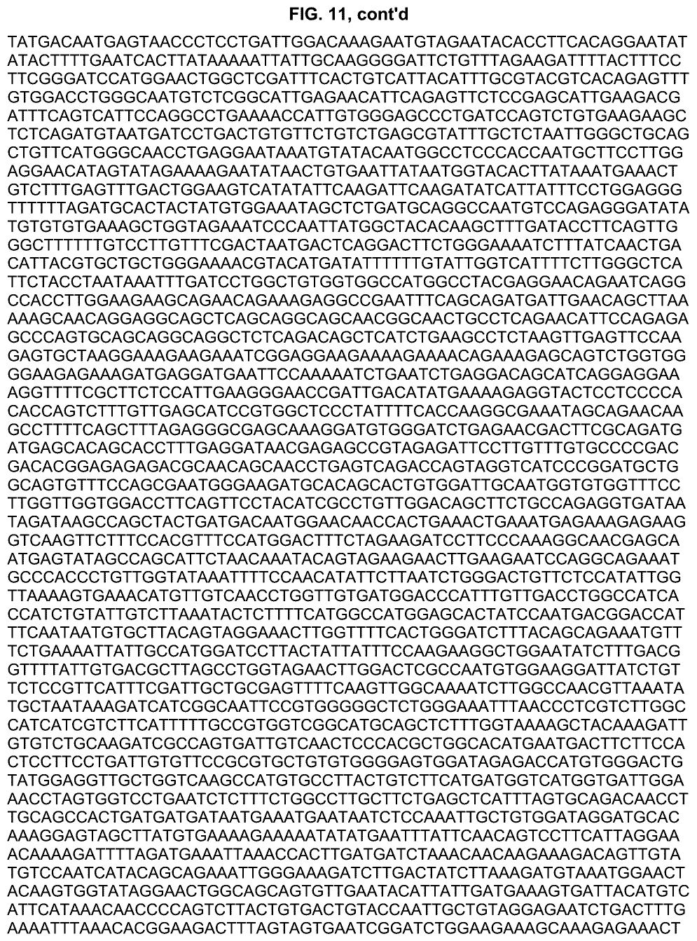

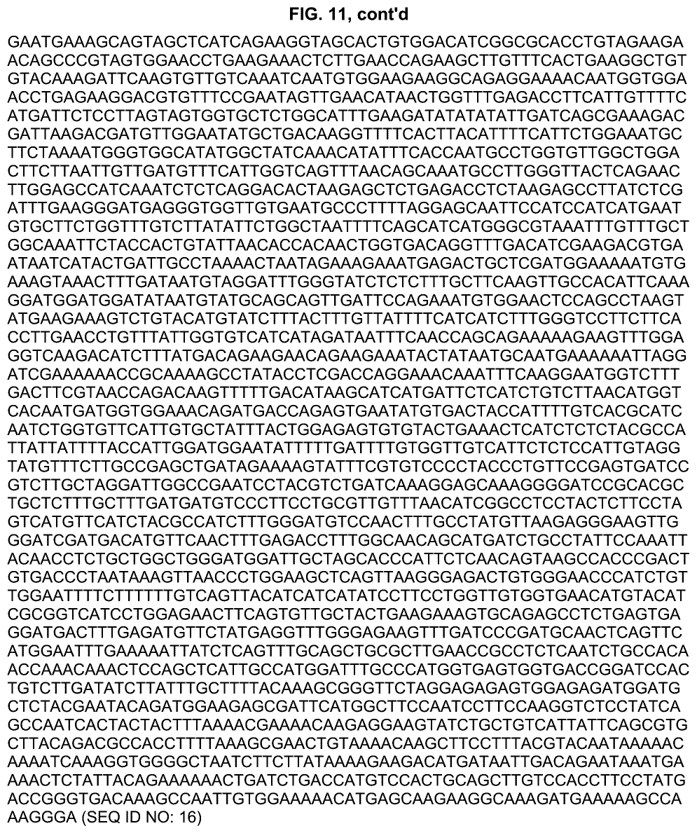

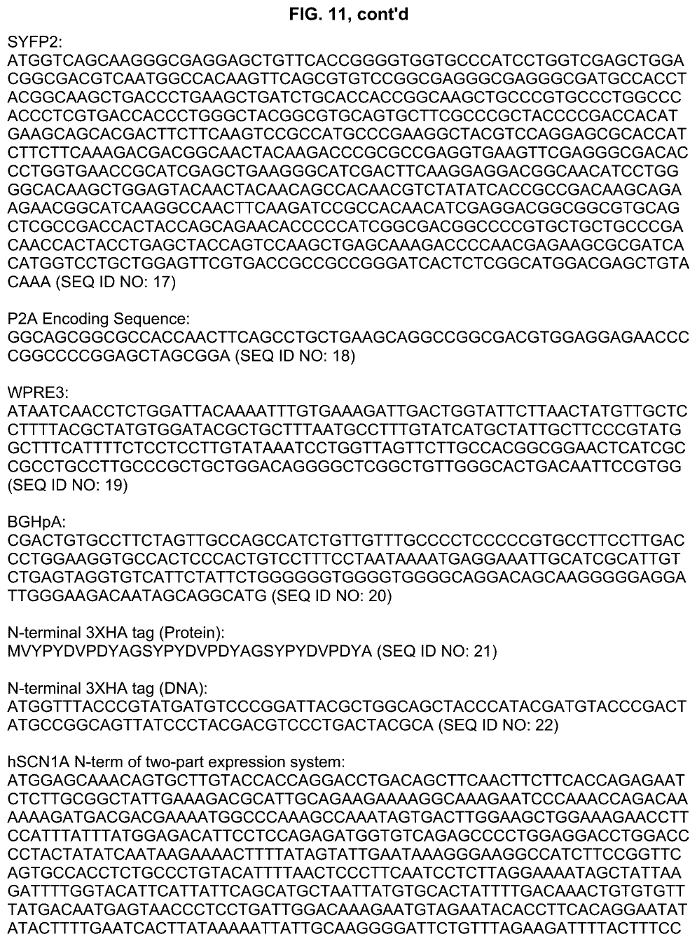

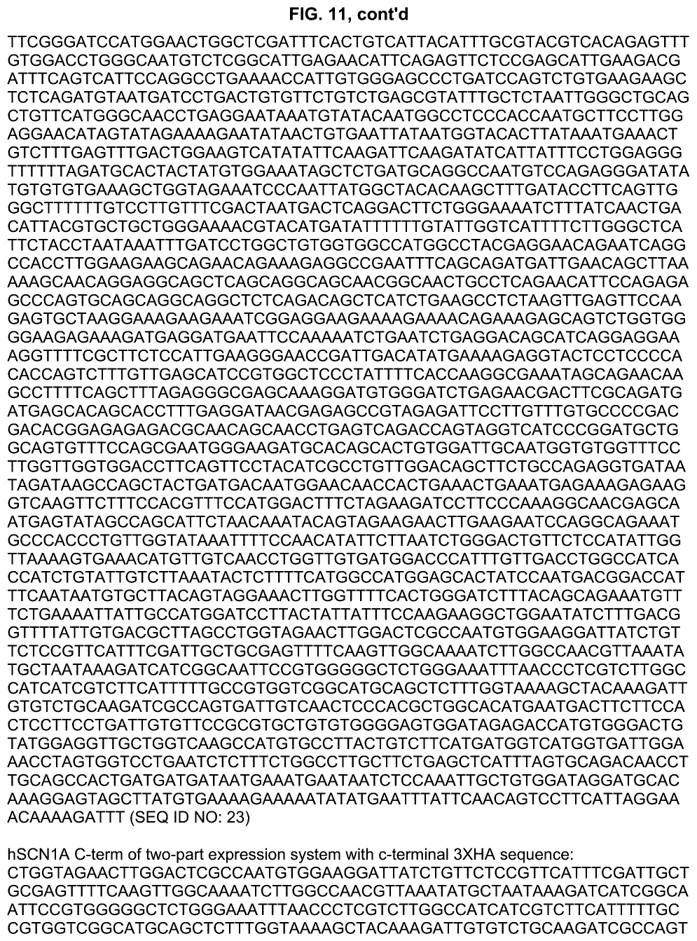

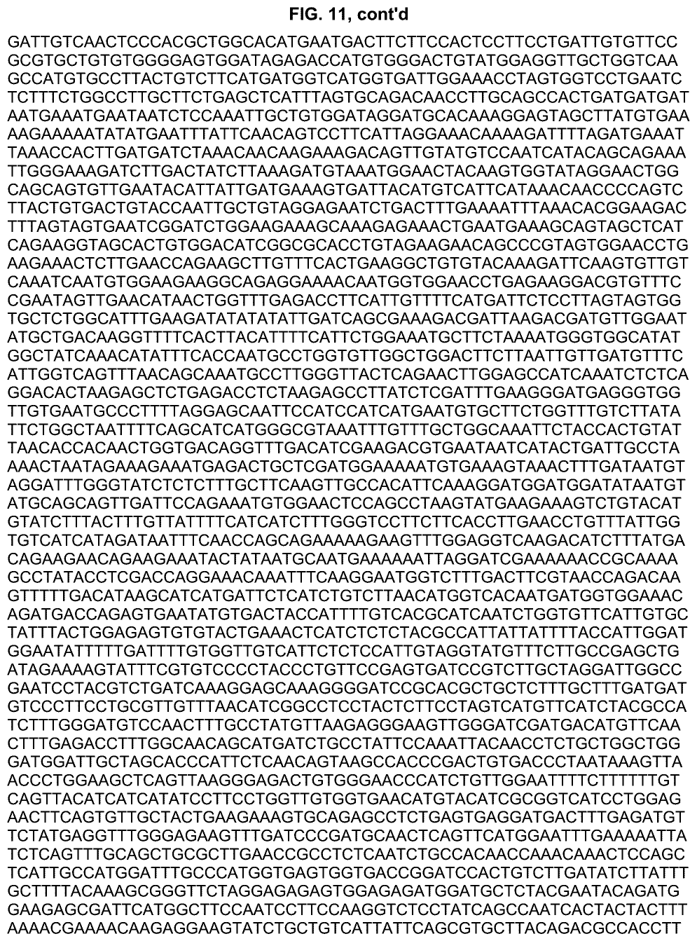

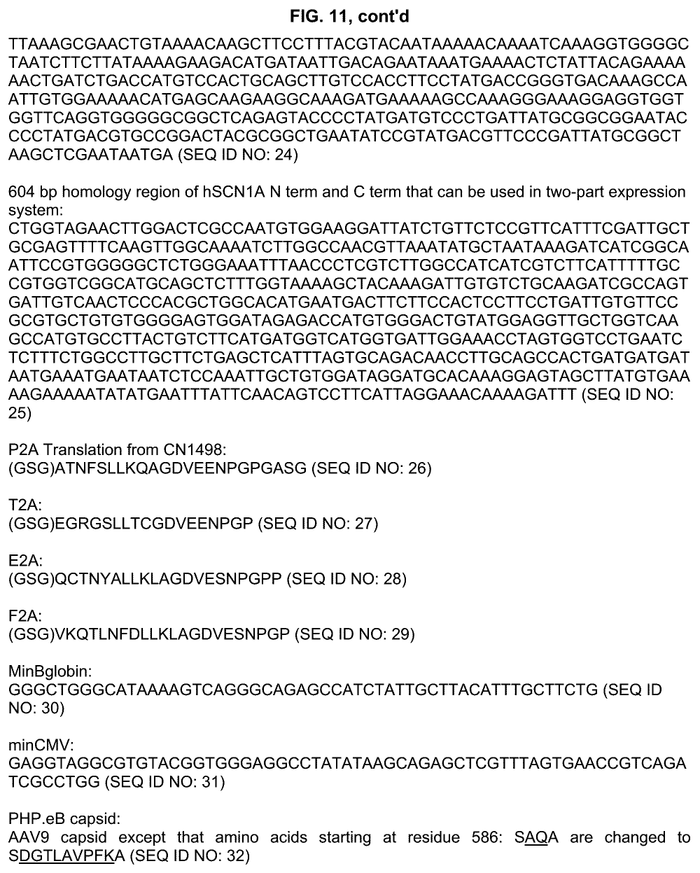

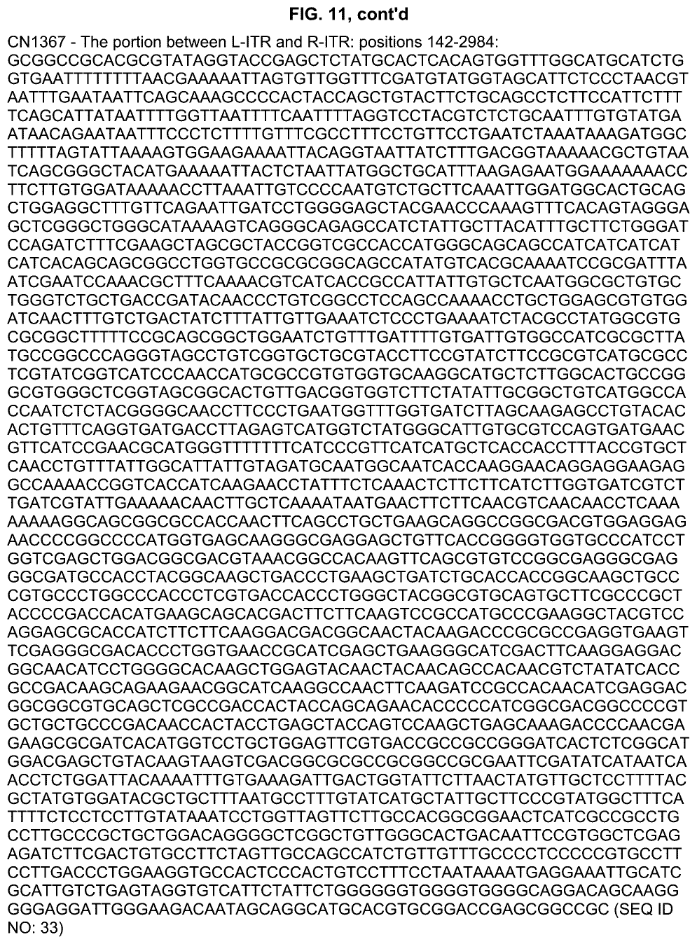

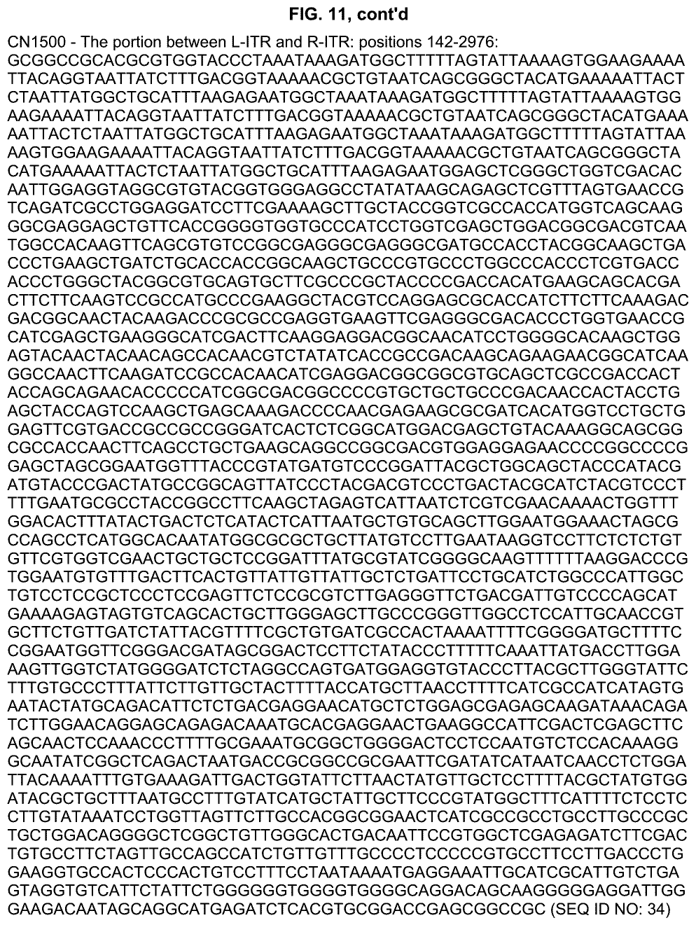

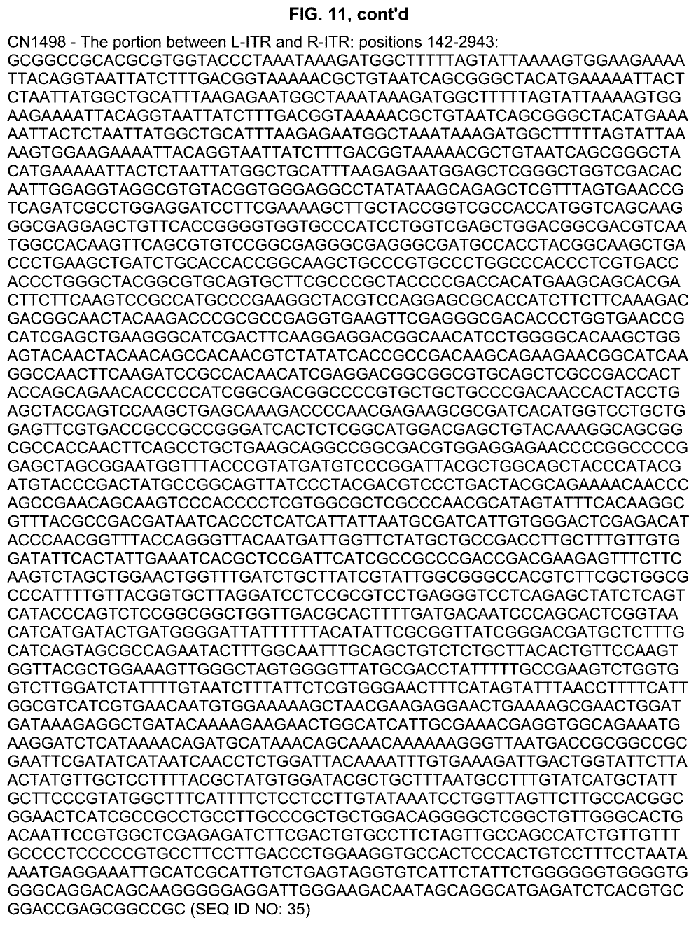

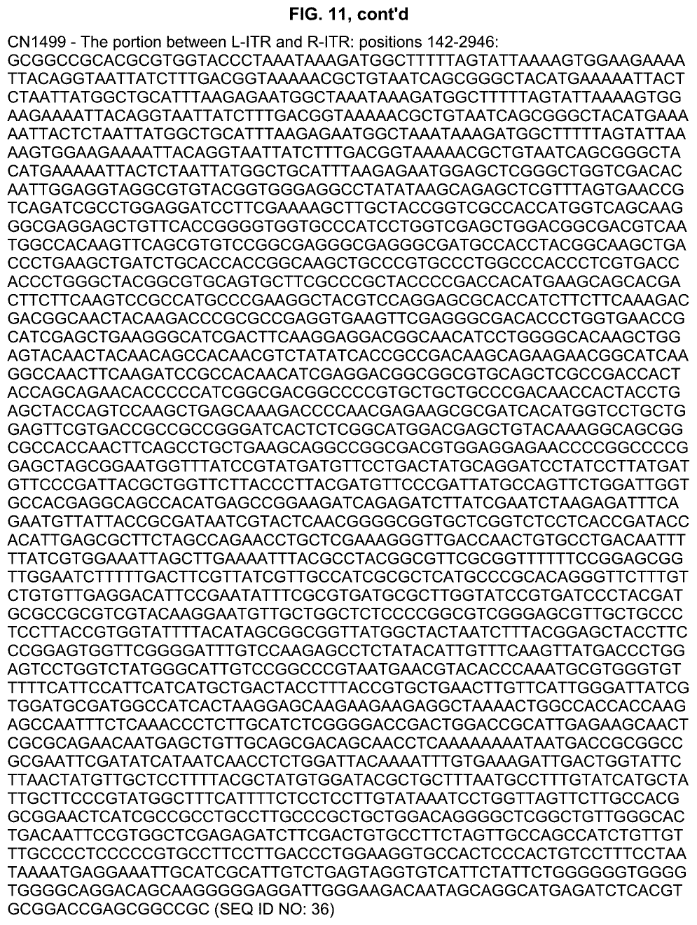

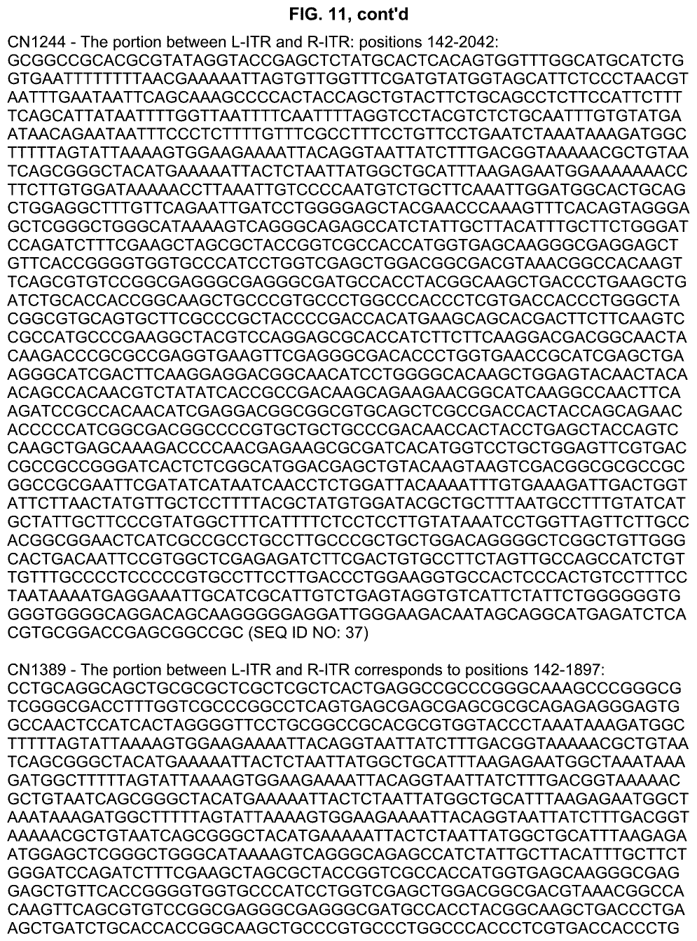

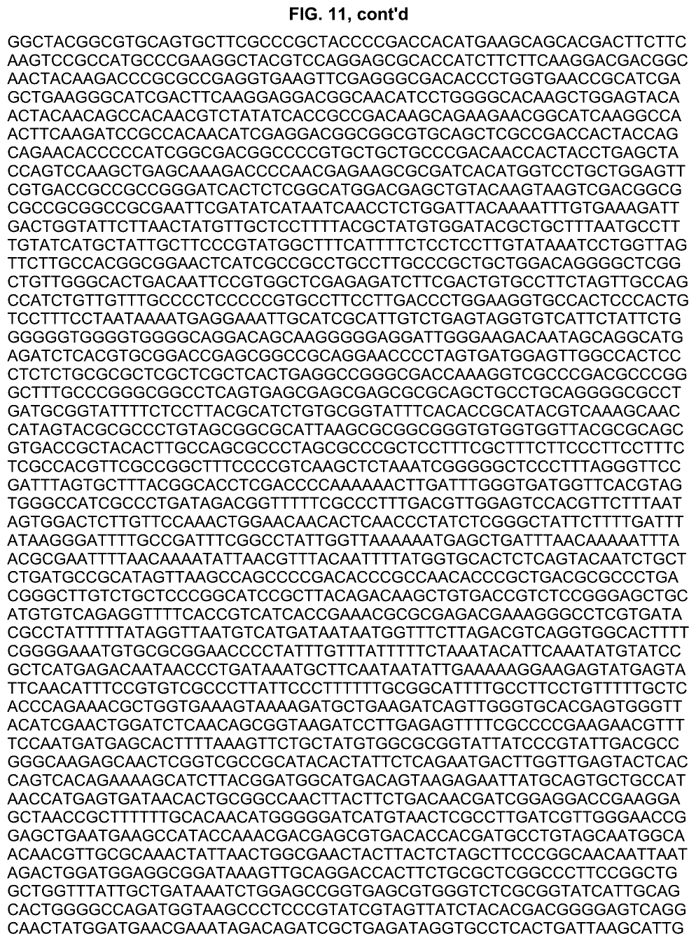

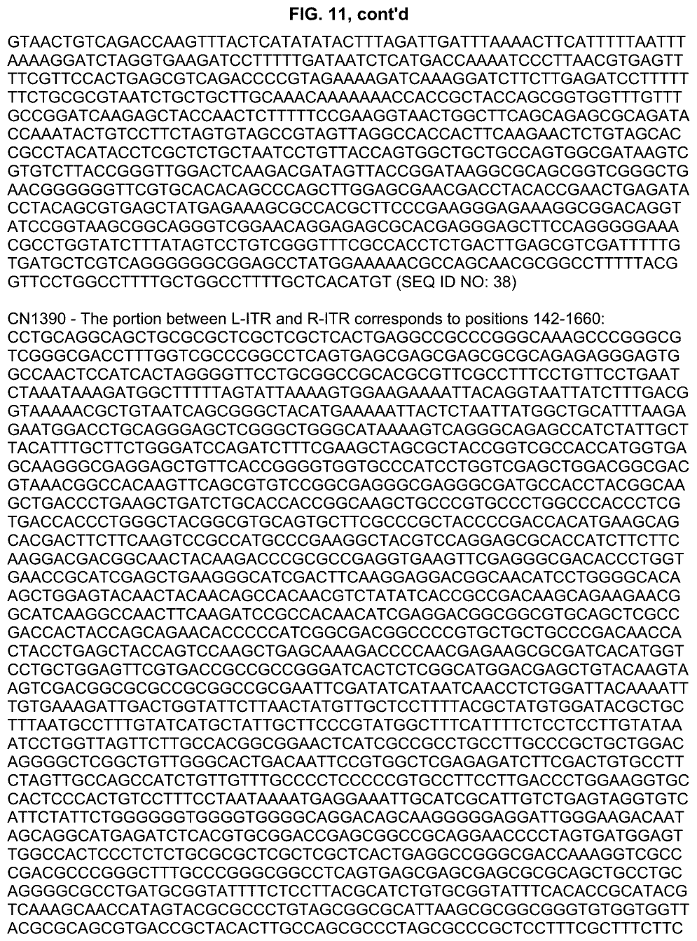

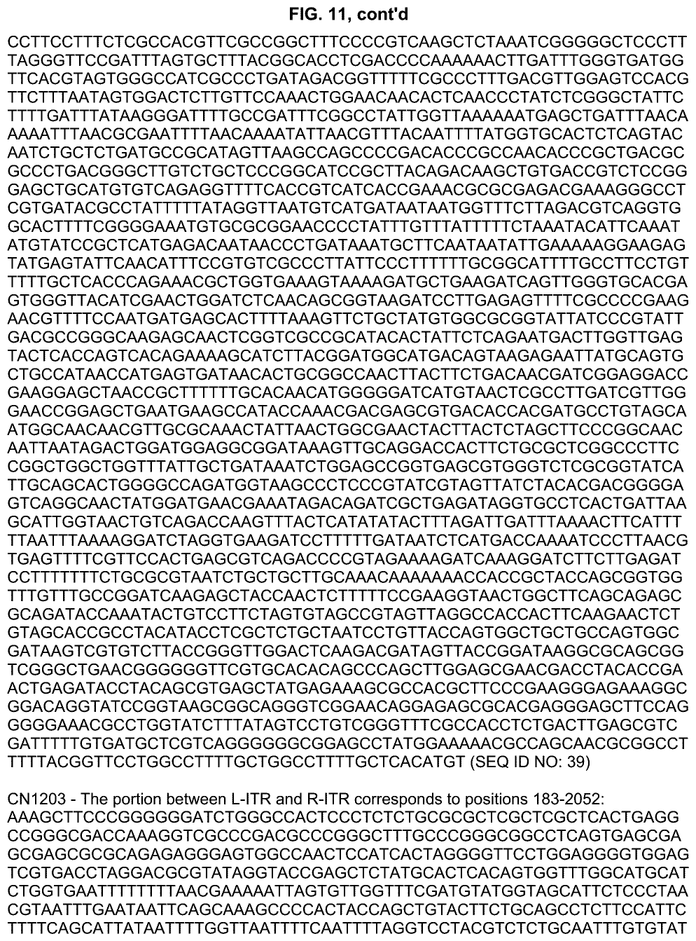

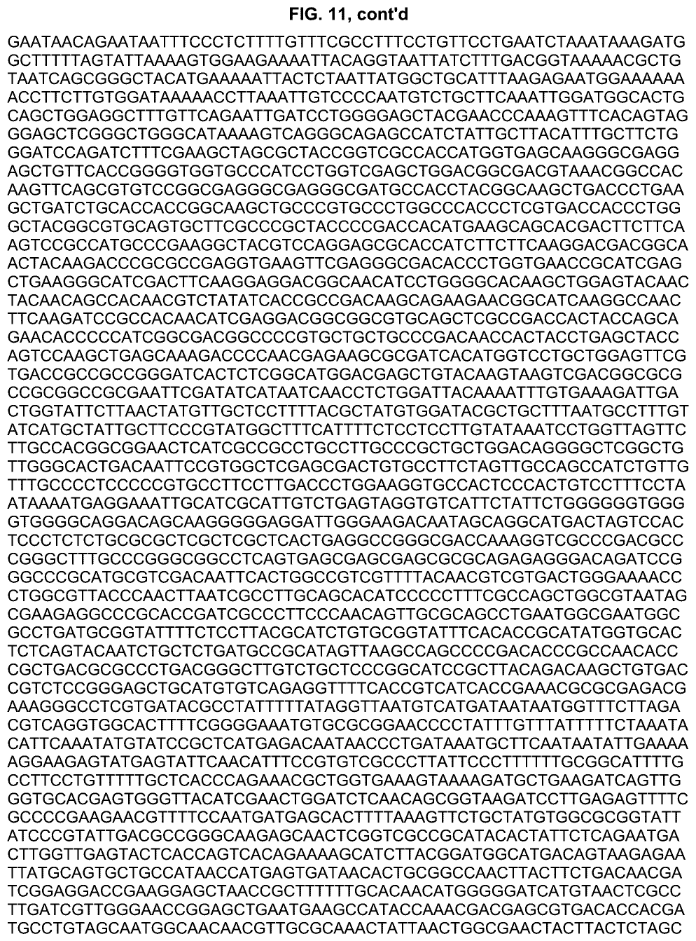

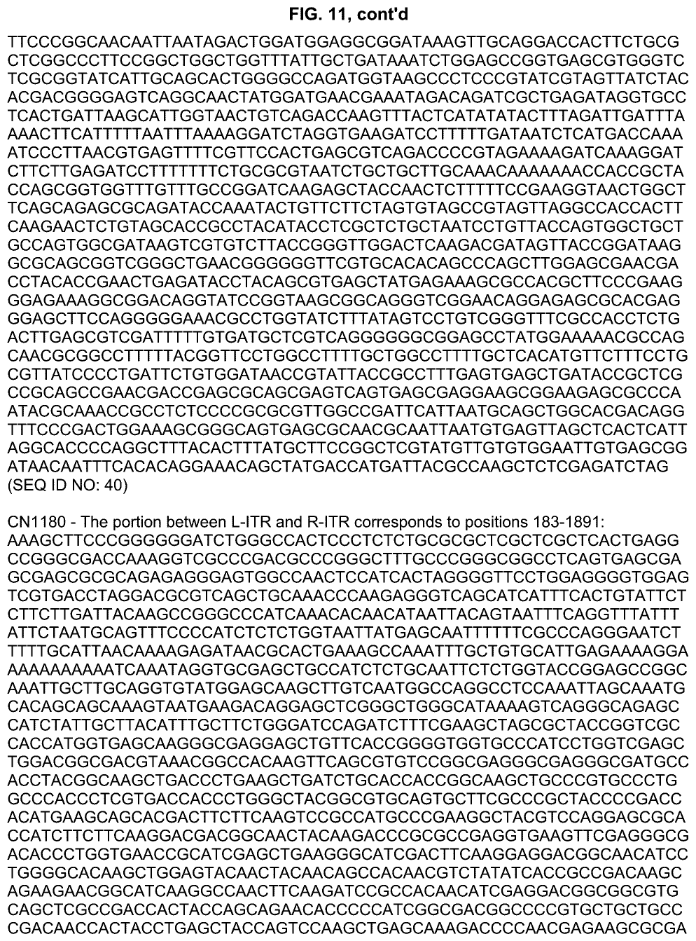

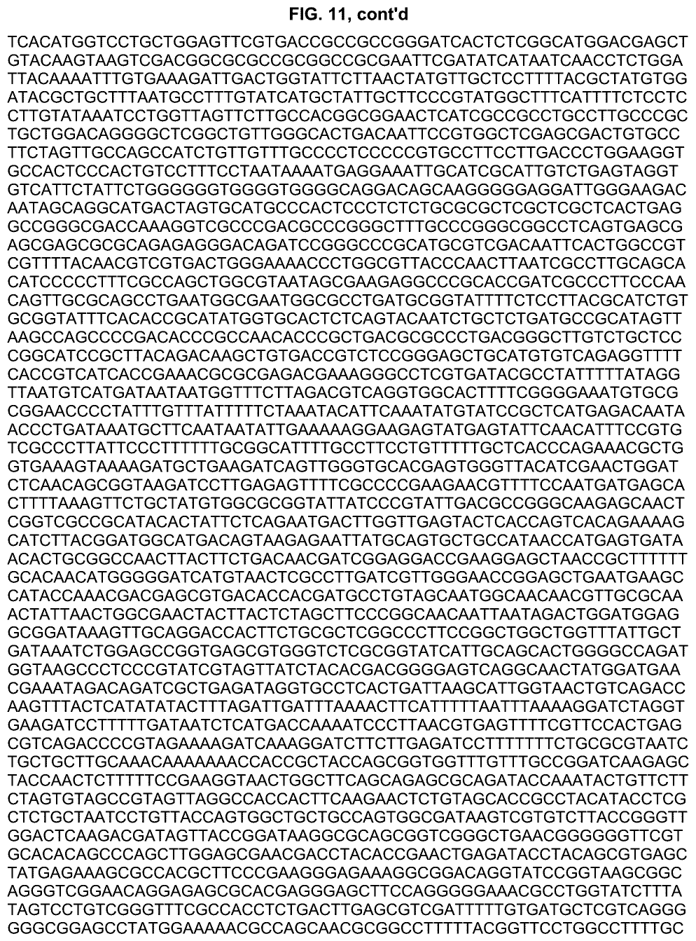

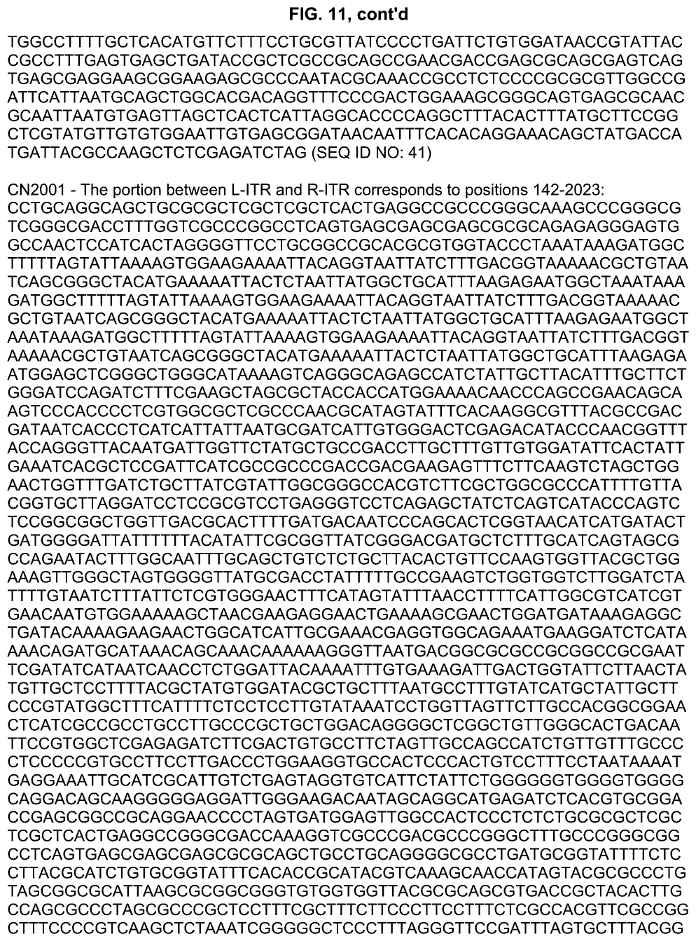

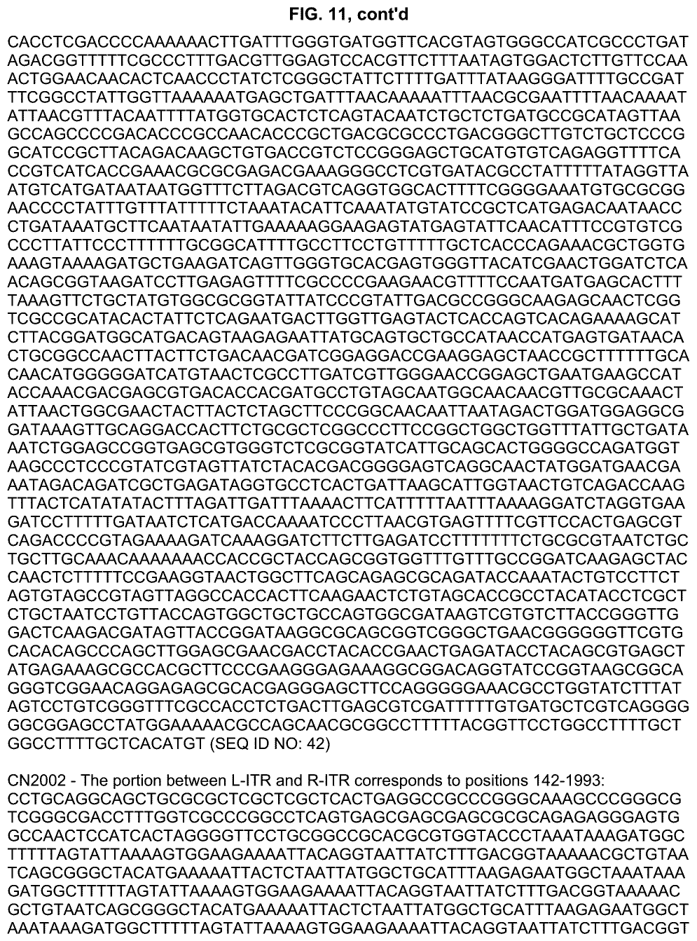

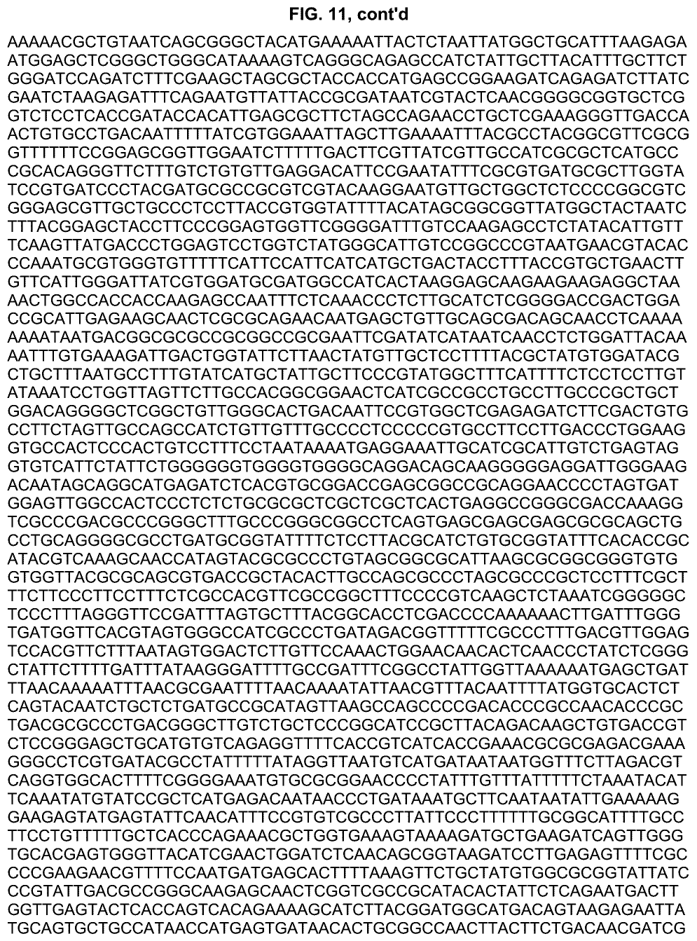

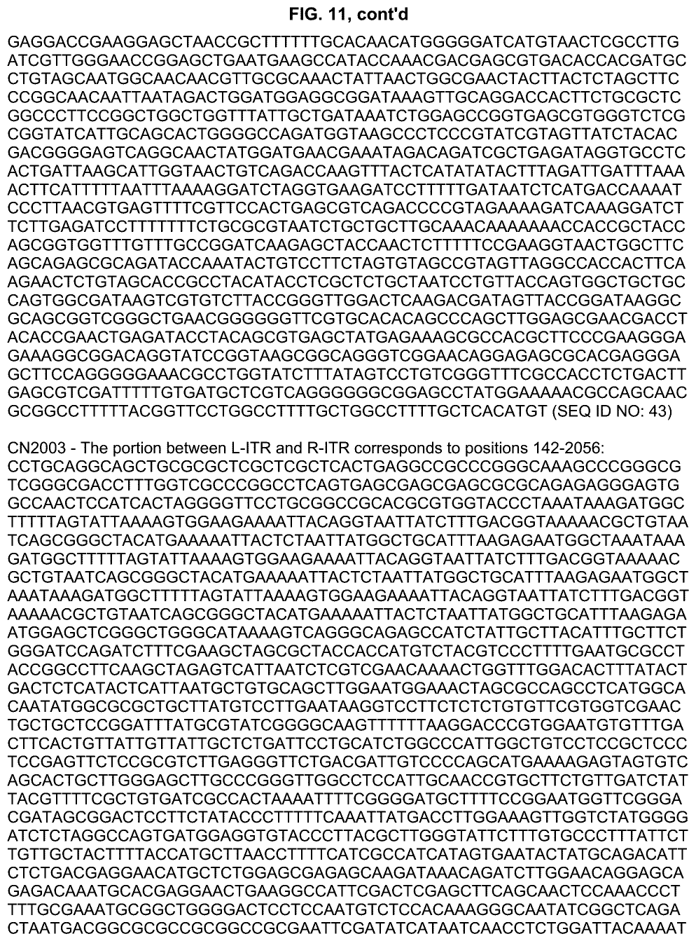

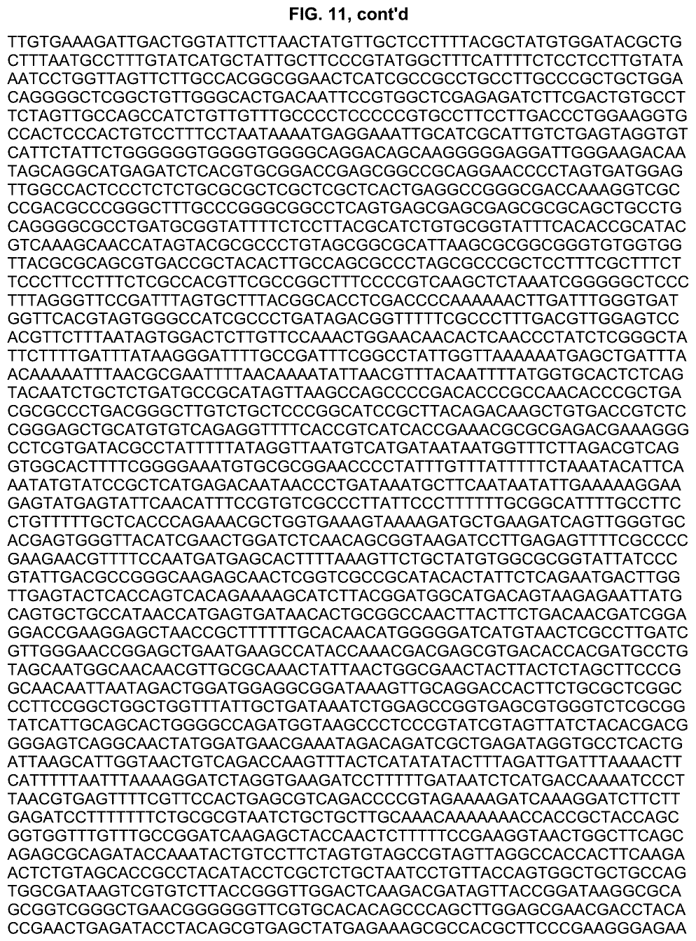

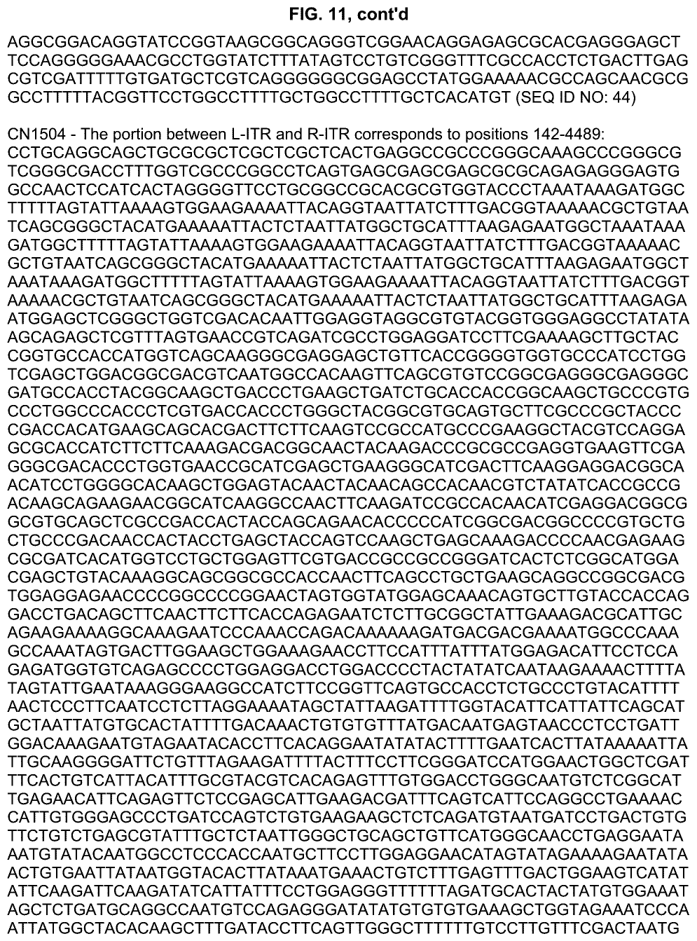

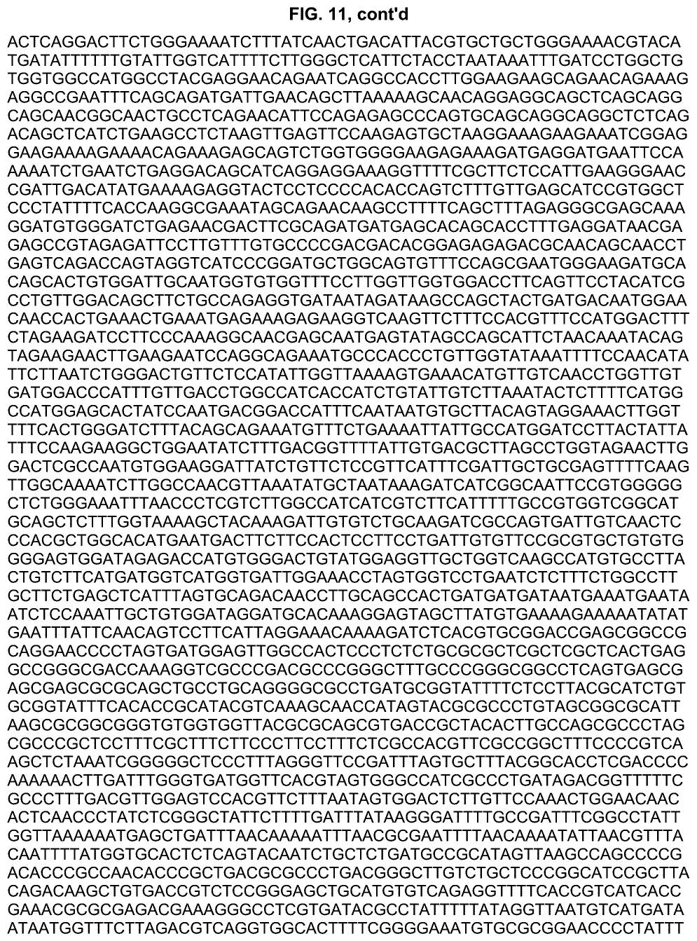

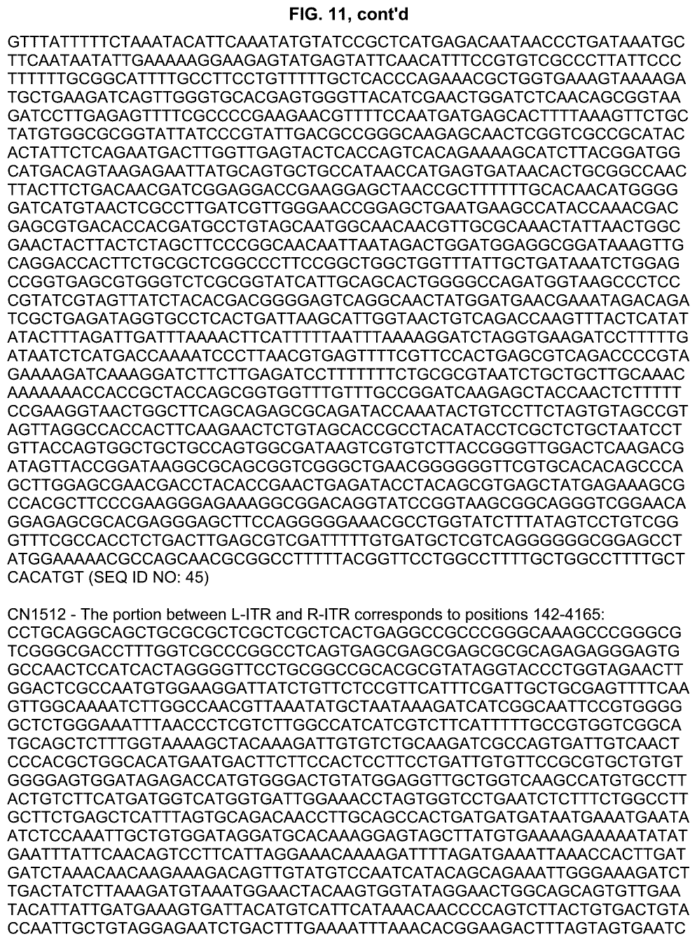

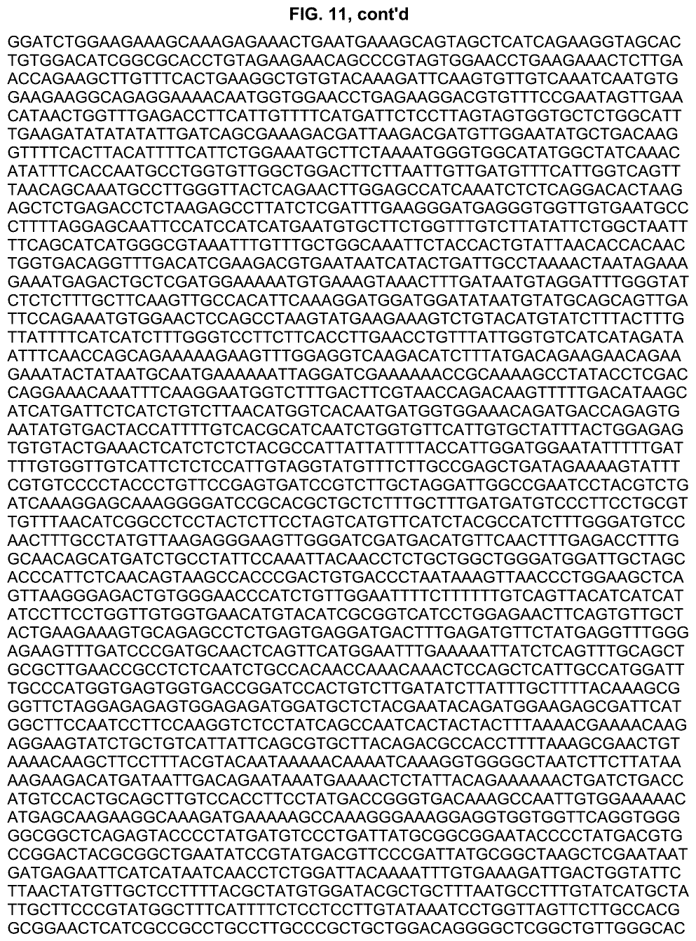

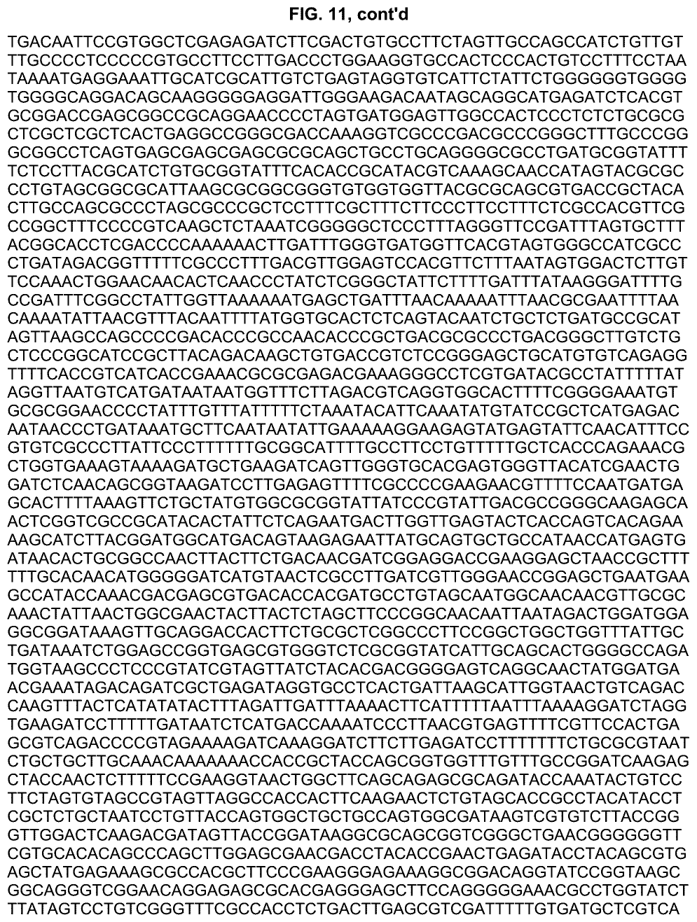

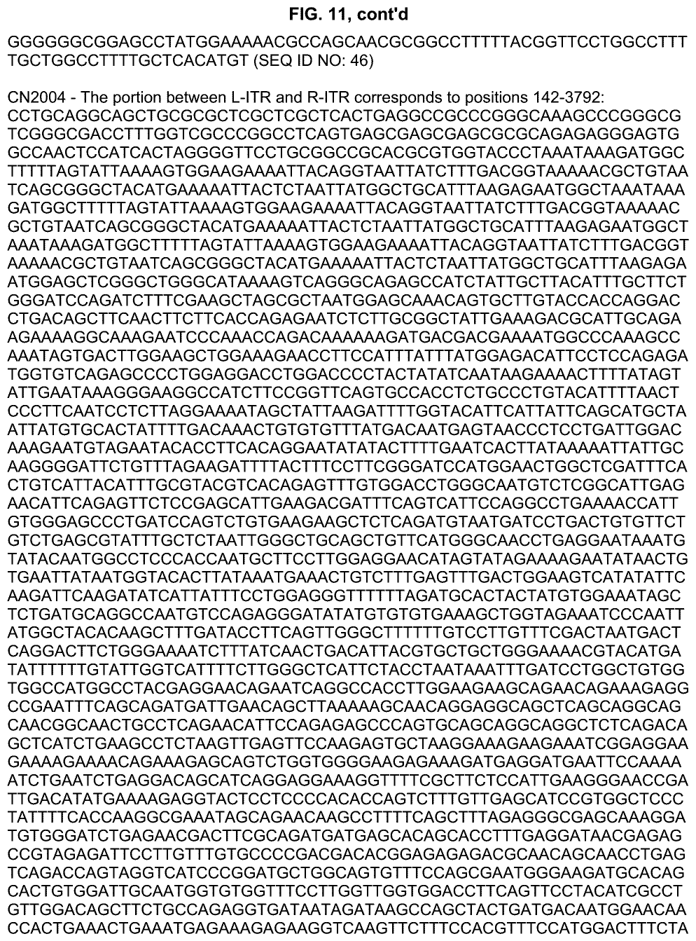

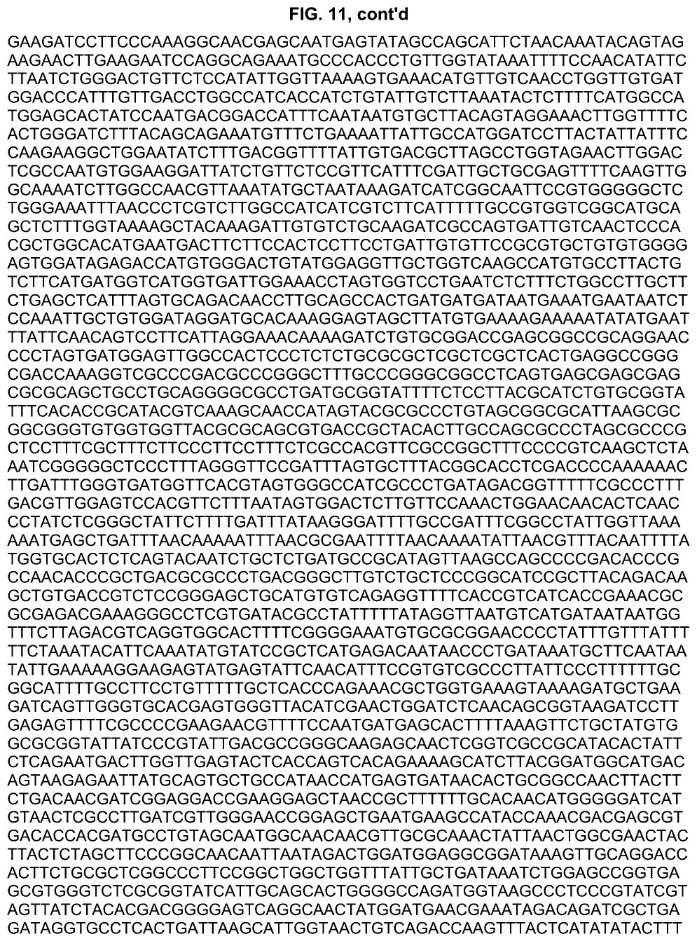

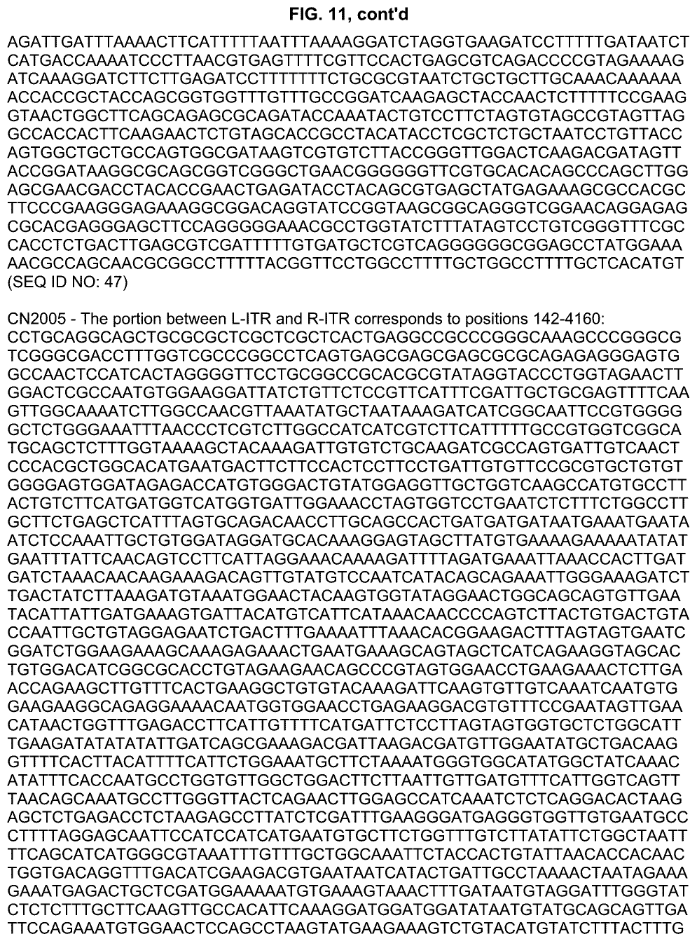

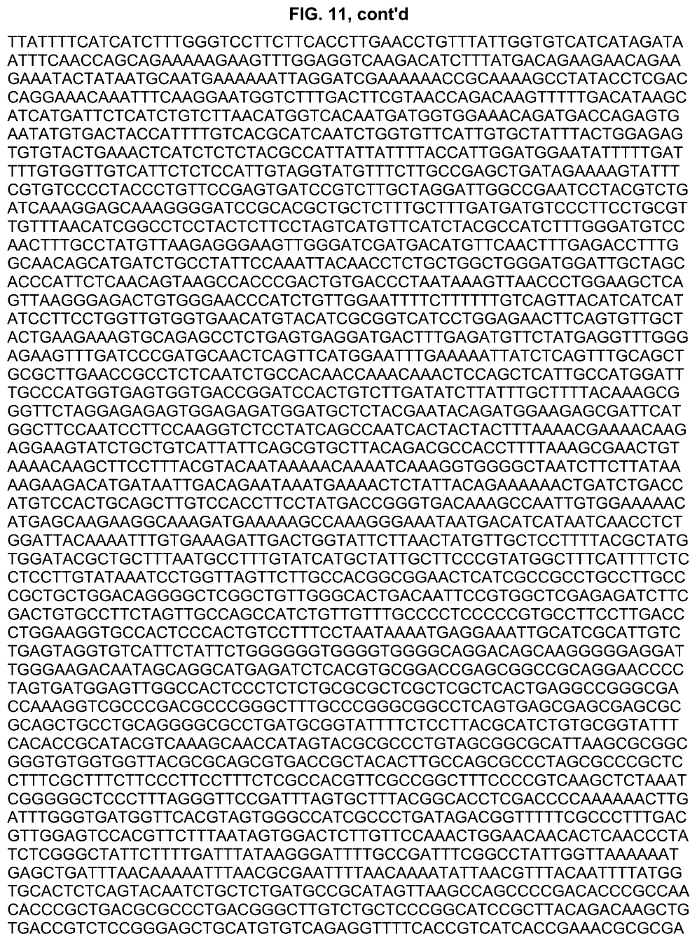

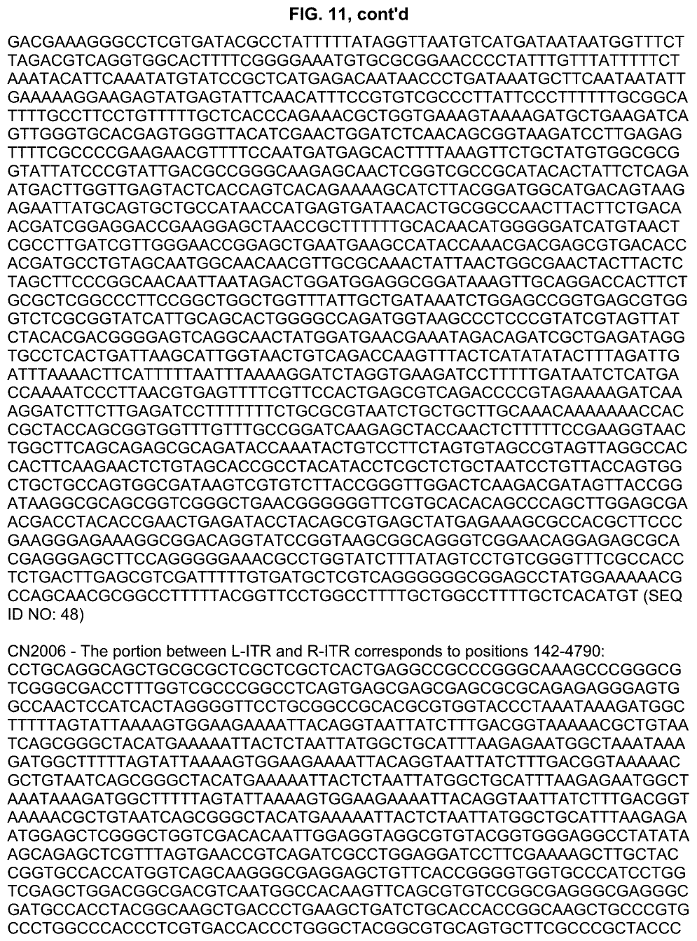

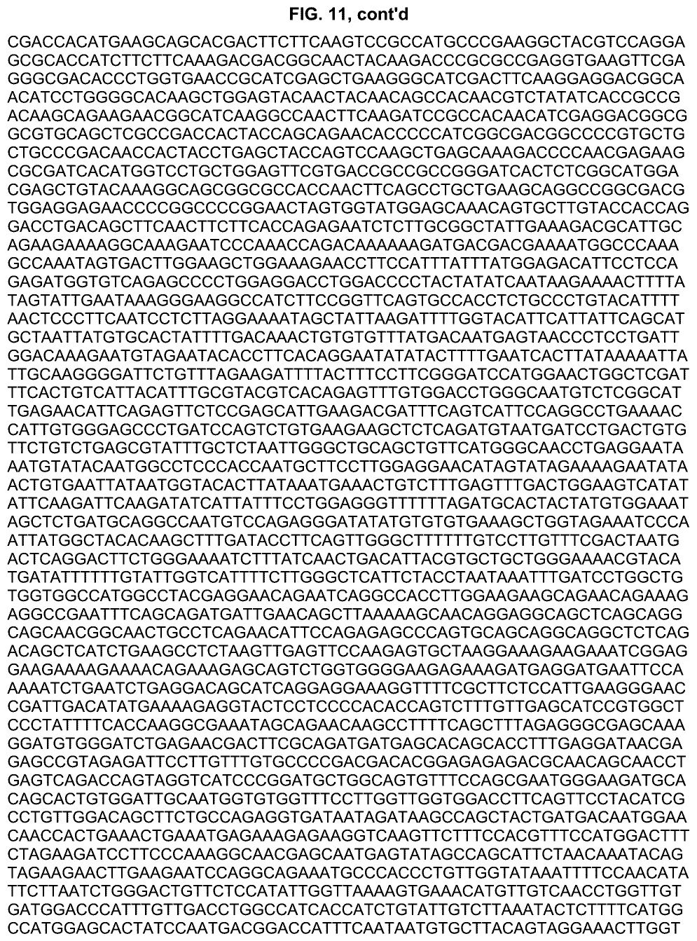

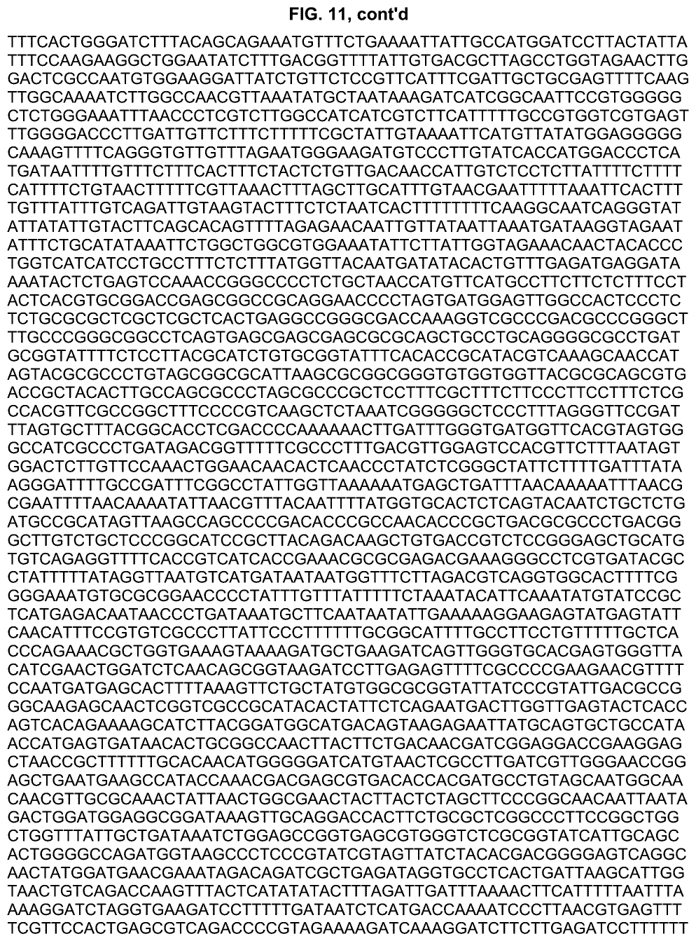

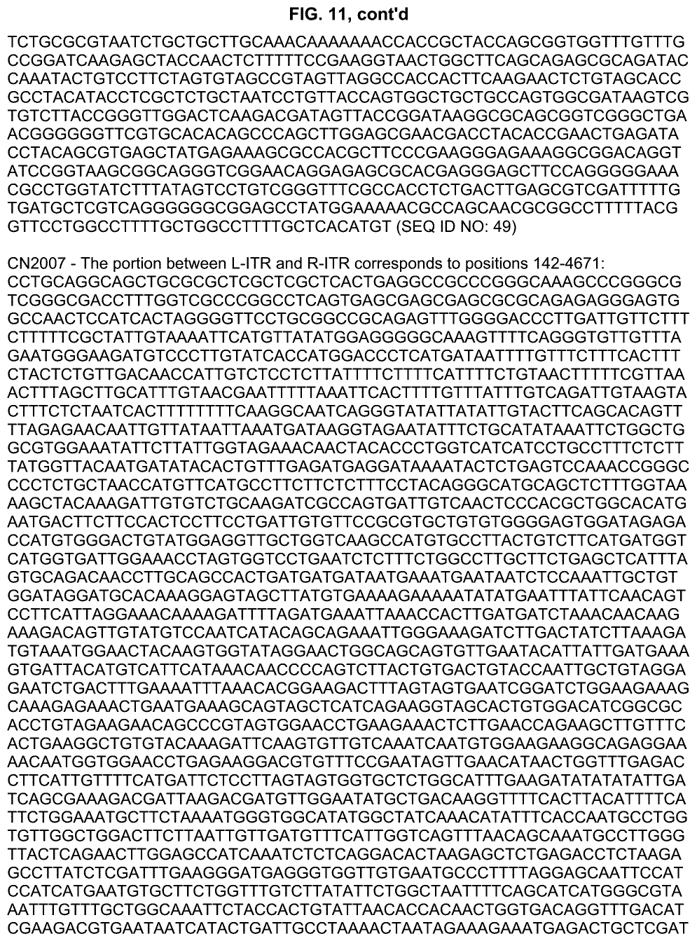

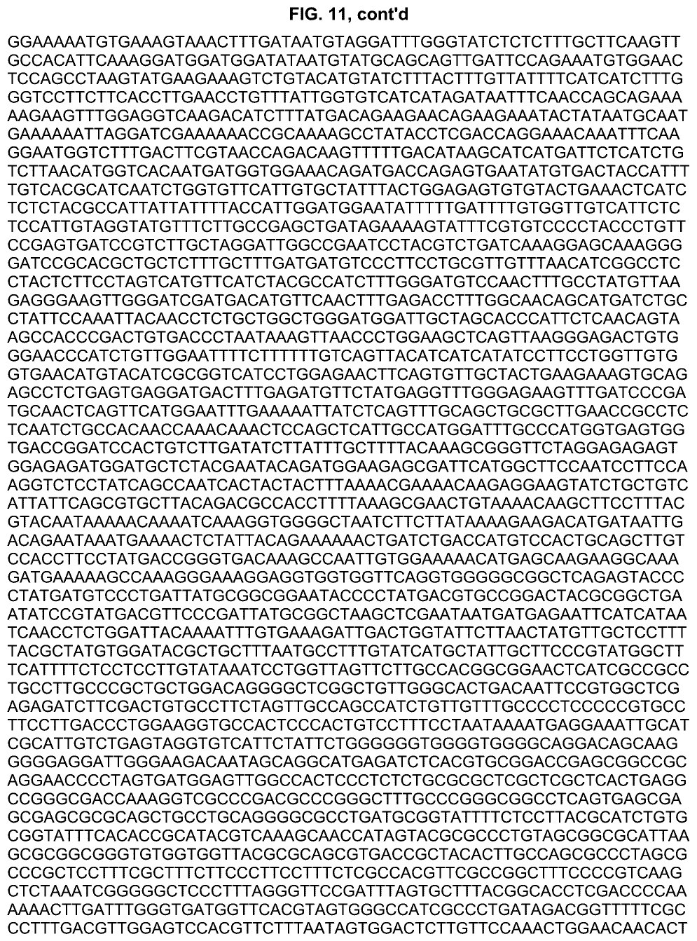

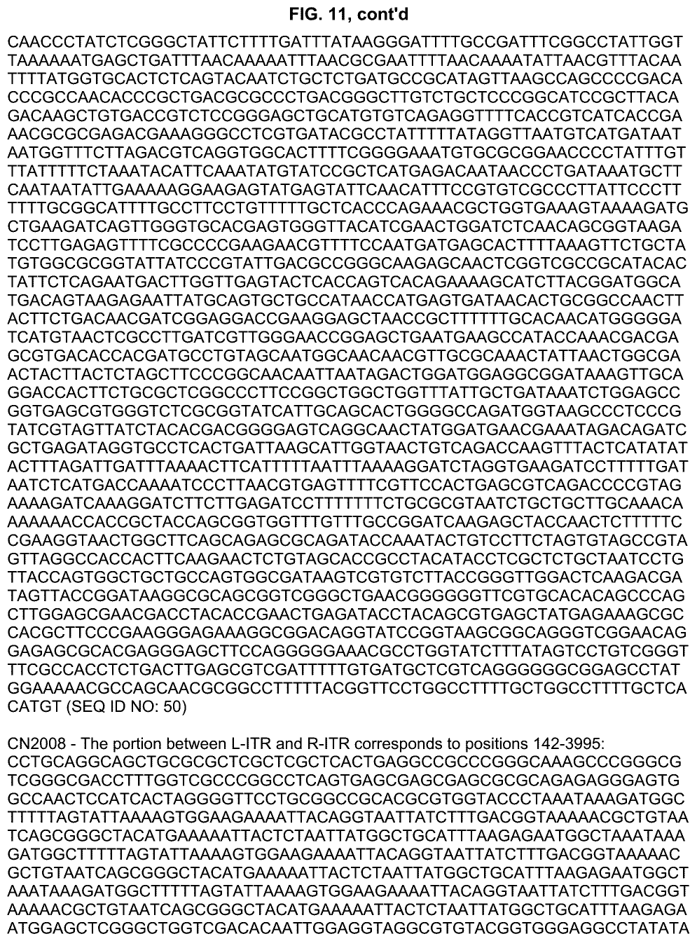

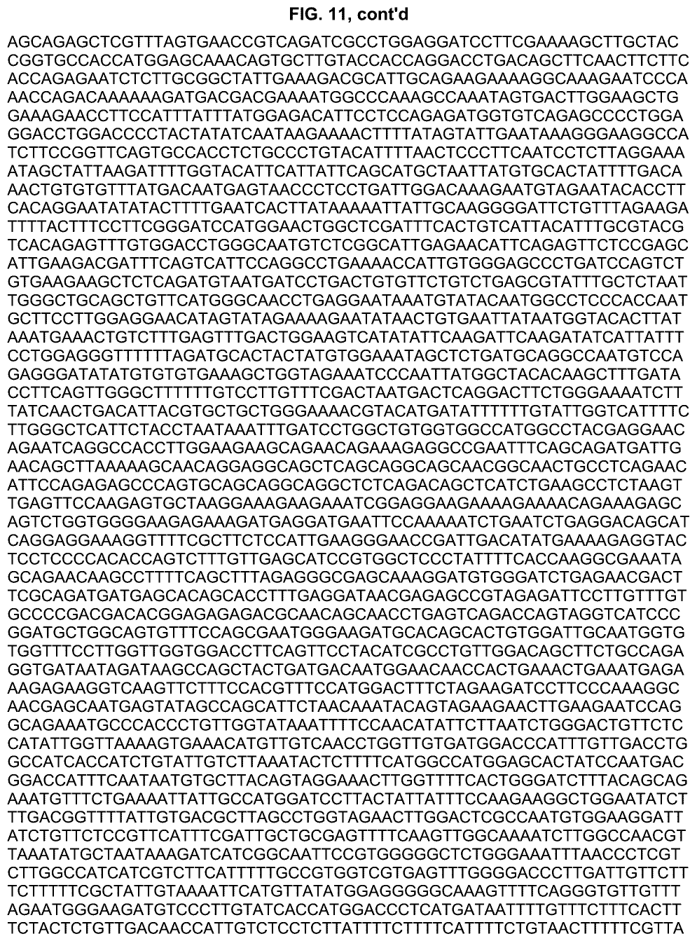

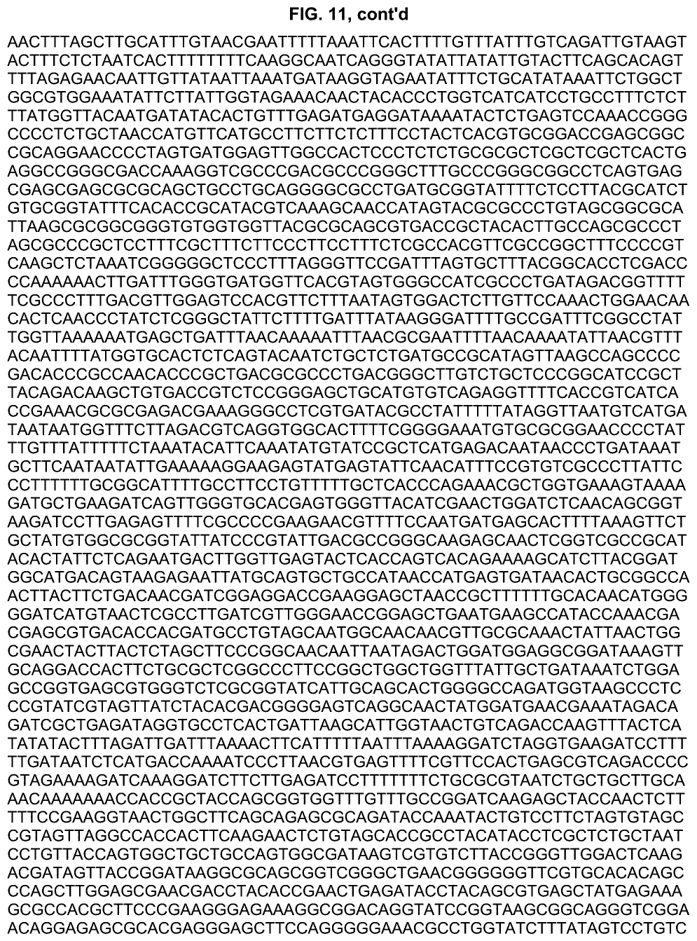

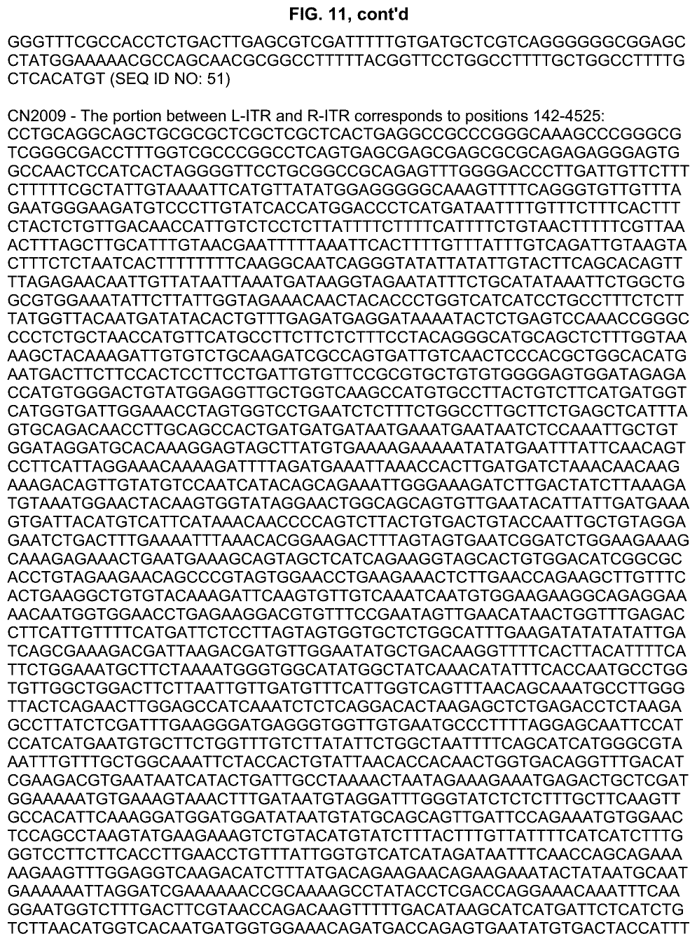

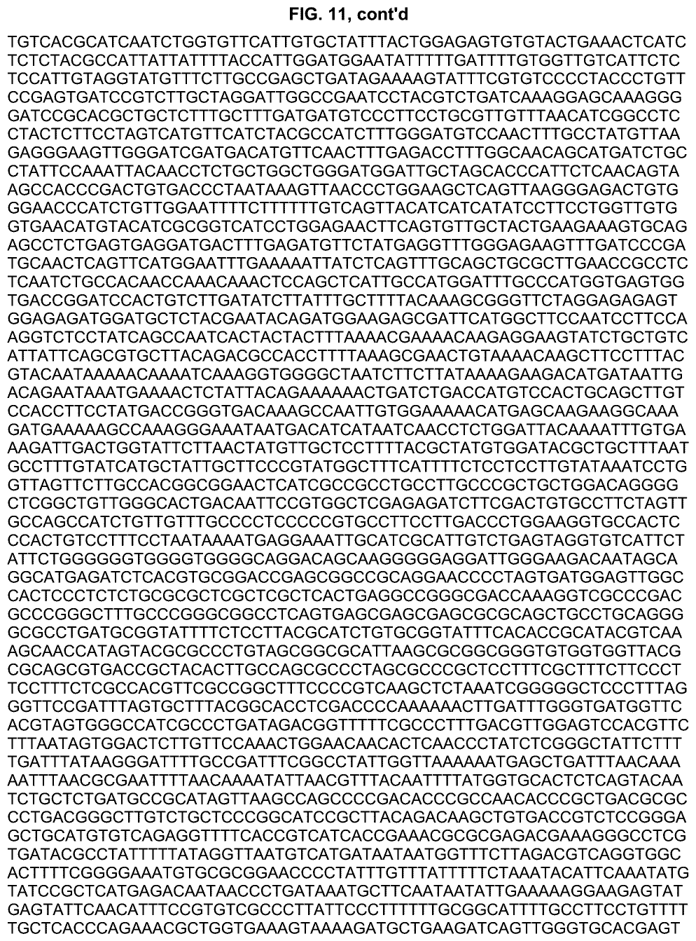

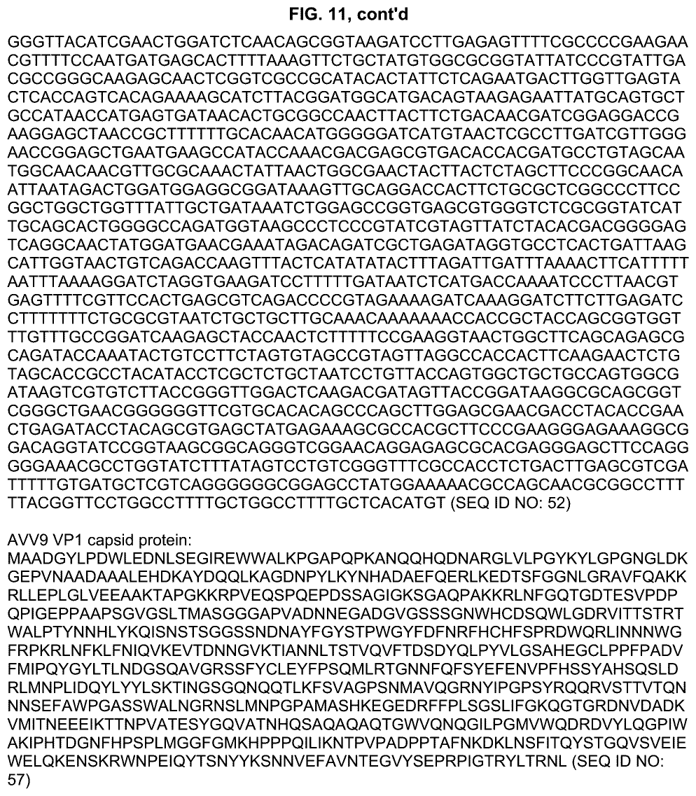

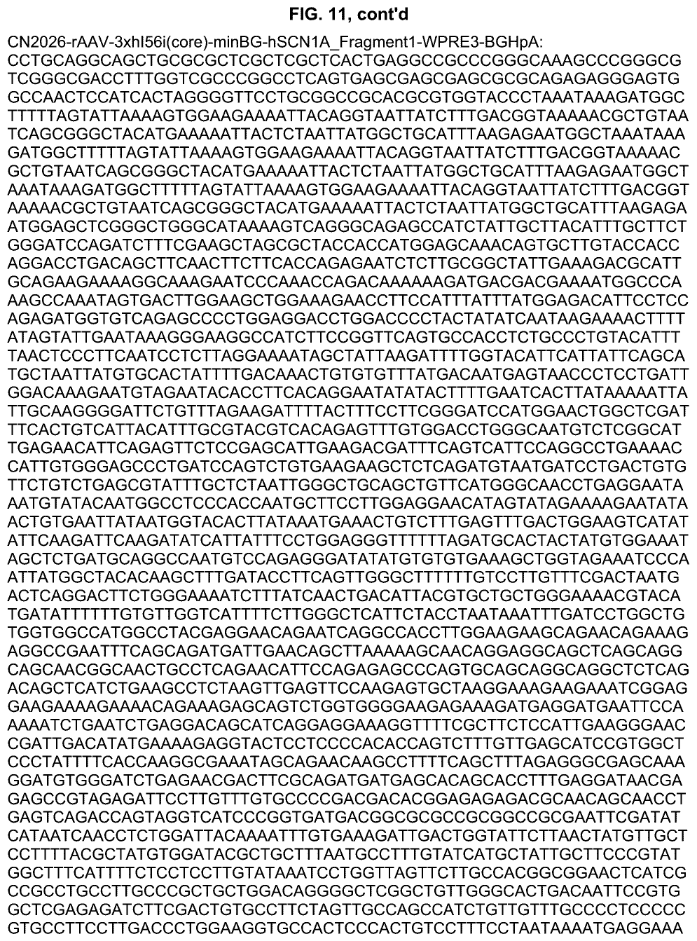

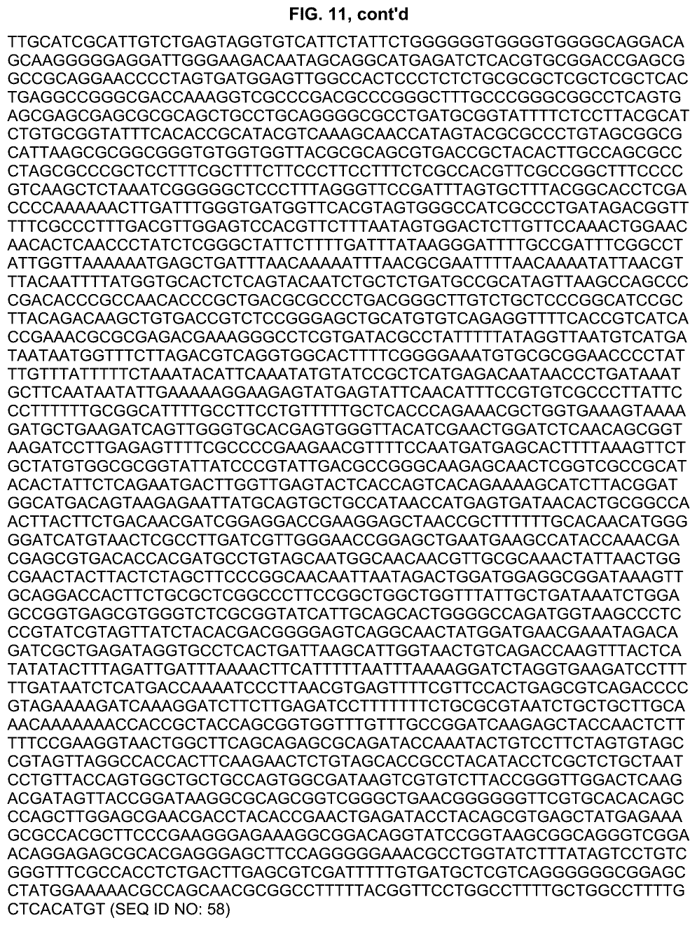

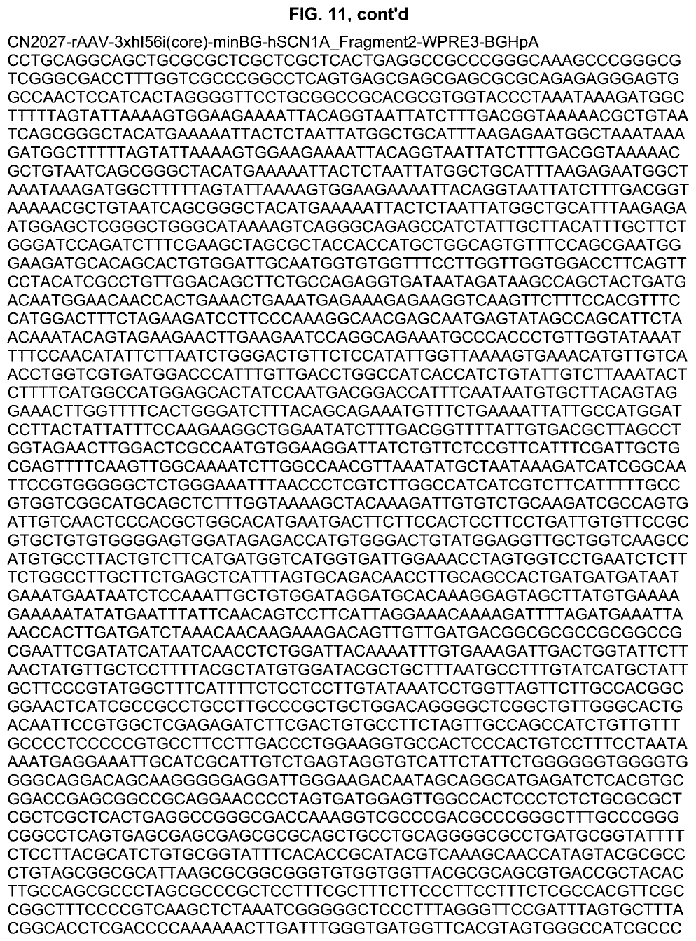

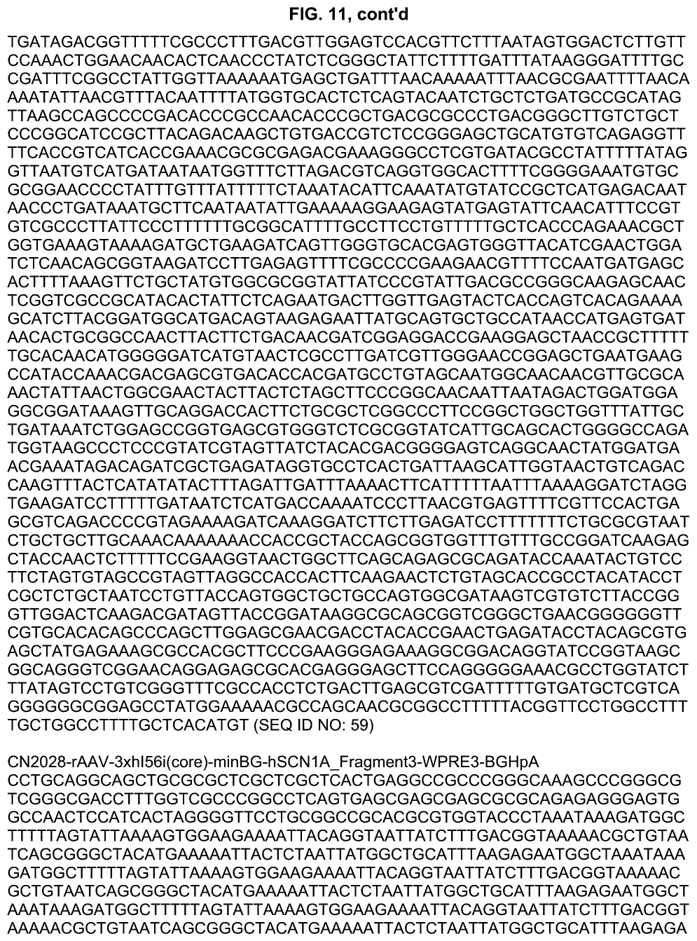

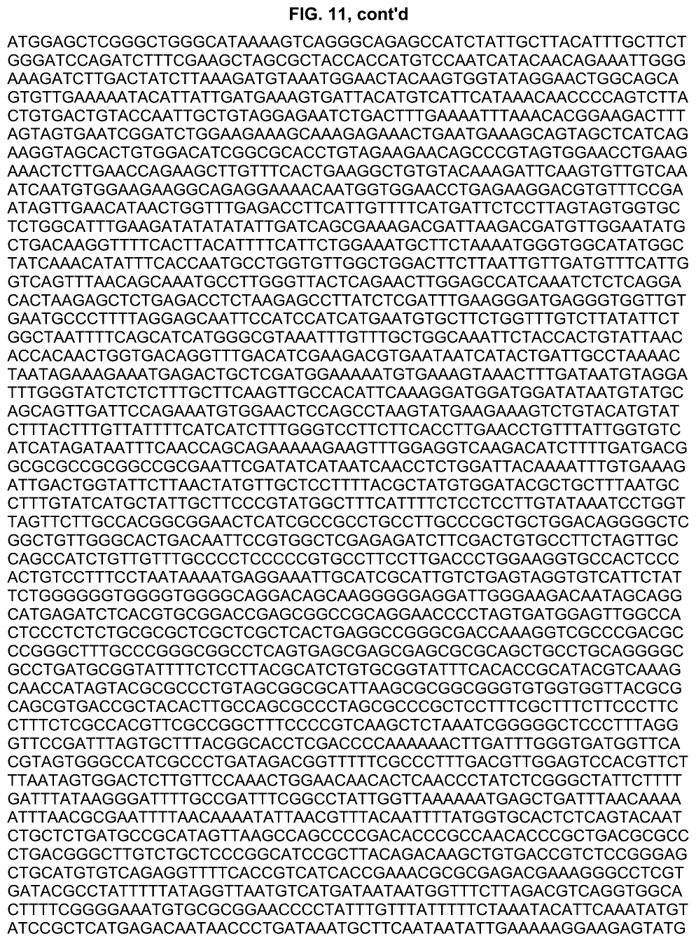

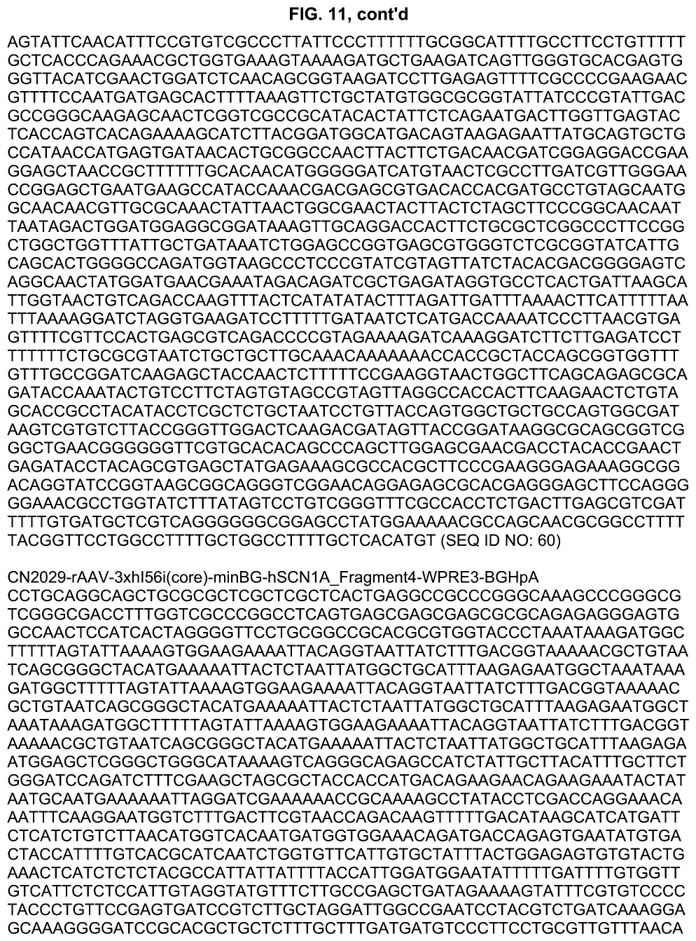

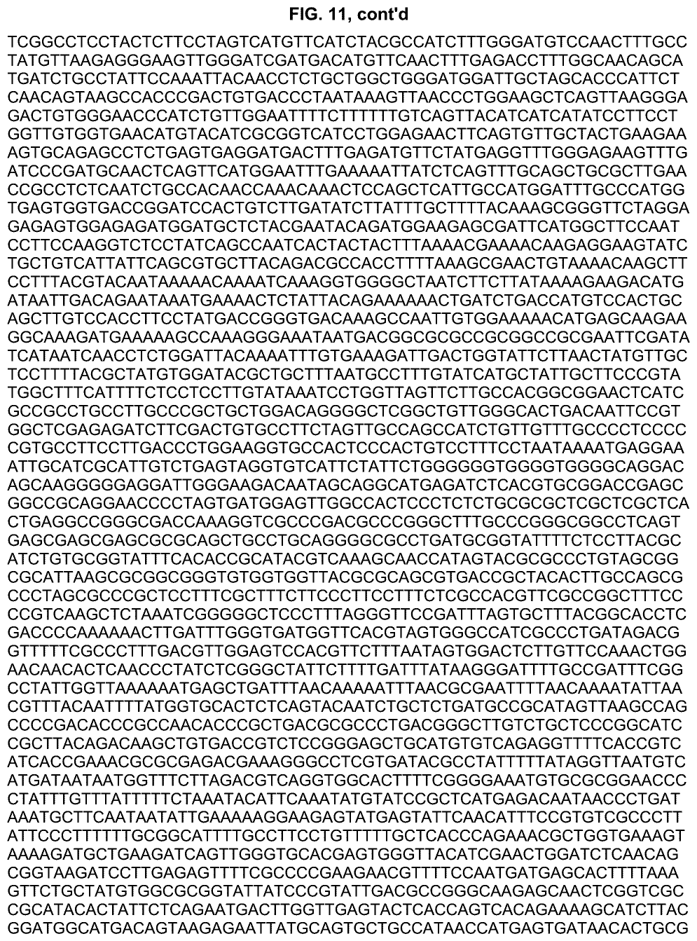

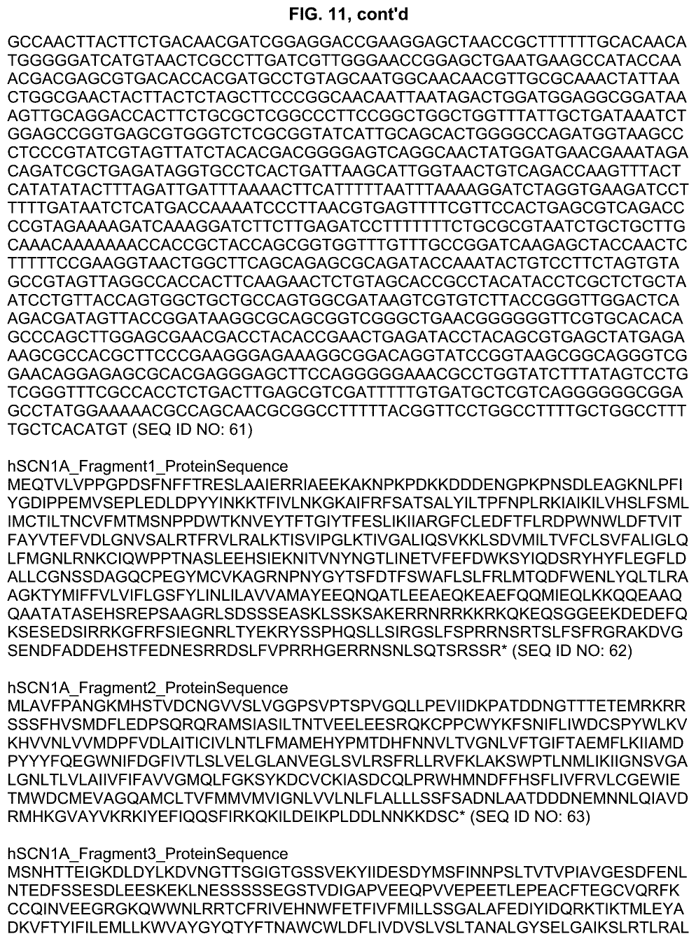

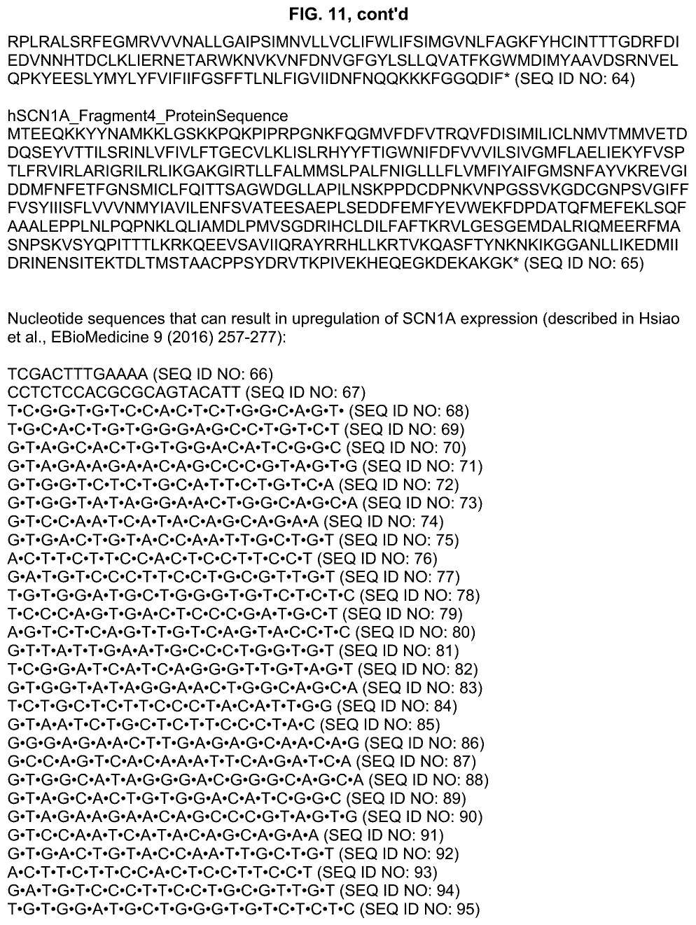

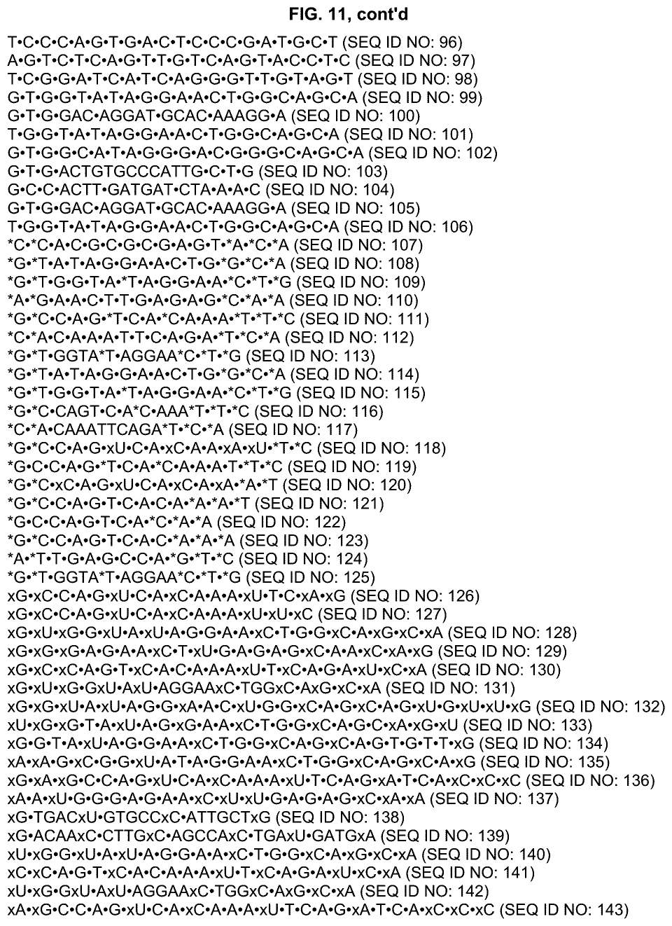

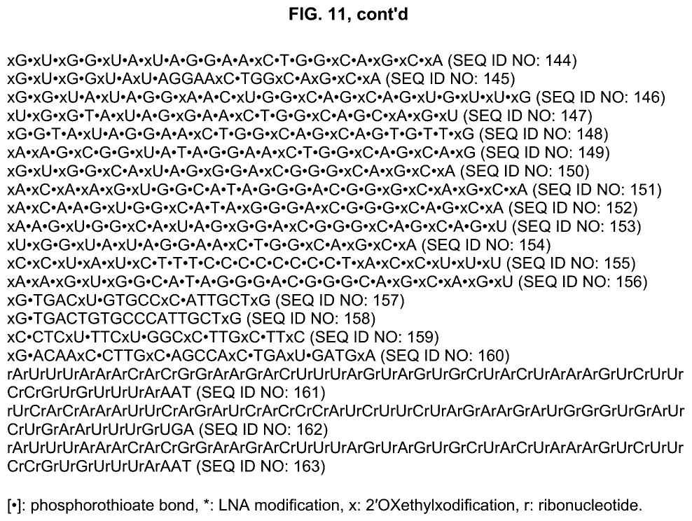

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a significantly significant reduction in the ability of a vector to reverse febrile seizures in Scn1a+/− mice according to the protocol of Example 2 and FIG. 10.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of treating Dravet Syndrome in a subject in need thereof comprising intravenously administering a therapeutically effective amount of at least two adeno-associated viral (AAV) vectors wherein each viral vector comprises an expression construct comprising
(i) an enhancer consisting of SEQ ID NO: 3;
(ii) a promoter; and
(iii) a coding sequence that results in expression of a fragment of human SCN1A wherein the AAV vectors are associated with a capsid that crosses the blood brain barrier, and wherein the human SCN1A fragments encoded by the coding sequences are expressed selectively within inhibitory GABAergic interneurons and result in rescue of Nav1.1 channel function within inhibitory GABAergic interneurons in need thereof following the intravenous administering
thereby treating Dravet Syndrome in the subject in need thereof.

2. The method of claim 1, wherein the viral vectors comprise SEQ ID NO: 47 and SEQ ID NO: 48; SEQ ID NO: 49 and SEQ ID NO: 50; or SEQ ID NO: 51 and SEQ ID NO: 52.

3. The method of claim 1, wherein the viral vectors comprise SEQ ID NO: 58, SEQ ID NO: 59, SEQ ID NO: 60, and SEQ ID NO: 61.

4. An expression construct comprising (i) an enhancer consisting of SEQ ID NO: 3; (ii) a promoter; and (iii) a coding sequence encoding a protein that rescues voltage-gated sodium channel function in a cell or subject in need thereof.

5. The expression construct of claim 4, wherein the coding sequence comprises NavSheP-D60N, NavBp, NavMs, 3×HA-NavSheP-D60N, 3×HA-NavBp, 3×HA-NavMs, His-NavMs, human SCN1A, mouse Scn1a, human SCN1A-3×HA, and/or mouse Scn1a-3×HA.

6. The expression construct of claim 4, wherein the promoter comprises minBglobin or minCMV.

7. The expression construct of claim 4, wherein the expression construct is within an adeno-associated viral (AAV) vector.

8. The expression construct of claim 4, wherein the expression construct comprises a coding sequence for a reporter protein.

9. The expression construct of claim 8, wherein the reporter protein comprises a fluorescent reporter protein.

10. The expression construct of claim 4, wherein the expression construct comprises or encodes a skipping element.

11. The expression construct of claim 10, wherein the skipping element comprises a 2A peptide or an internal ribosome entry site (IRES).

12. The expression construct of claim 11, wherein the 2A peptide comprises T2A, P2A, E2A, and/or F2A.

13. The expression construct of claim 7, wherein the construct comprises the elements of CN1367, CN1244, CN1389, CN1390, CN1180, CN1203, CN1498, CN1499, CN1500, CN2001, CN2002, CN2003, CN1504 and CN1512, CN2004 and CN2005, CN2006 and CN2007, or CN2008 and CN2009.

14. The expression construct of claim 7, wherein the construct comprises SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and/or SEQ ID NO: 52.

15. The expression construct of claim 7, wherein the construct comprises SEQ ID NO: 45 and SEQ ID NO: 46, SEQ ID NO: 47 and SEQ ID NO: 48, SEQ ID NO: 49 and SEQ ID NO: 50, or SEQ ID NO: 51, and SEQ ID NO: 52.

16. The expression construct of claim 7, wherein the AAV vector is associated with a capsid that crosses the blood brain barrier.

17. The expression construct of claim 16, wherein the capsid comprises PHP.Eb.

18. The expression construct of claim 16, wherein the capsid comprises SEQ ID NO: 53.

19. The expression construct of claim 16, wherein the capsid comprises AAV9 capsid with a SEQ ID NO: 54 or SEQ ID NO: 55 insert.

20. The expression construct of claim 16, wherein the capsid comprises AAV2 capsid with a SEQ ID NO: 56 insert.

21. A composition comprising the expression construct of claim 4.

22. A cell comprising the expression construct of claim 4.

23. A non-human animal comprising the expression construct of claim 4.

24. A kit comprising the expression construct of claim 4.

25. A method of rescuing voltage-gated sodium channel function in a defective cell in need thereof comprising administering a therapeutically effective amount of a composition of claim 21 to the cell.

26. A method of rescuing voltage-gated sodium channel function in a subject in need thereof comprising administering a therapeutically effective amount of a composition of claim 21 to the subject.

27. The method of claim 26, wherein the subject is need thereof due to a diagnosis of epilepsy, an SCN1A-related seizure disorder, simple febrile seizures (FS), generalized epilepsy with febrile seizures plus (GEFS+), Dravet Syndrome (DS), intractable childhood epilepsy with generalized tonic-clonic seizures (ICE-GTC), intractable infantile partial seizures, myoclonic-astatic epilepsy, Lennox-Gastaut syndrome (LGS), or infantile spasms.

28. The method of claim 26, wherein the subject is a pediatric patient.

29. The method of claim 26, wherein the subject is less than 4 years old.

30. The method of claim 26, wherein the subject is a transgenic Scn1a^{+/-} mouse undergoing a temperature-induced febrile seizure test.

31. The method of claim 26, wherein the composition is administered intravenously.

32. The method of claim 26, wherein the composition is administered intrathecally into cerebrospinal fluid, via the lateral ventricles or cisterna magna or lumbar space or cannula into the foramen magnum.

33. An expression construct comprising:
(a) a non-naturally occurring enhancer sequence; and
(b) a nucleic acid encoding a protein that rescues voltage-gated sodium channel function; wherein the enhancer sequence:
(i) consists of the sequence of SEQ ID NO: 3;
(ii) comprises two or more copies of SEQ ID NO: 2 arranged in tandem;
(iii) comprises a sequence having at least 90% sequence identity with SEQ ID NO: 3 and maintaining interneuron-specific enhancer function; or
(iv) a sequence the complementary strand of which is capable of hybridizing to the sequence of (i), (ii), or (iii); and
wherein the enhancer promotes the transcription of the nucleic acid selectively within inhibitory neurons following administration to a sample or subject.

34. The expression construct of claim 33, wherein nucleic acid is selected from NavSheP-D60N, NavBp, NavMs, 3×HA-NavSheP-D60N, 3×HA-NavBp, 3×HA-NavMs, His-NavMs, human SCN1A, mouse Scn1a, human SCN1A-3×HA, or mouse Scn1a-3×HA.

35. The expression construct of claim 33, comprising a promoter comprising minBglobin or minCMV.

36. The expression construct of claim 33, wherein the expression construct is within an adeno-associated viral (AAV) vector.

37. The expression construct of claim 33, wherein the expression construct comprises a nucleic acid encoding a reporter protein.

38. The expression construct of claim 37, wherein the reporter protein comprises a fluorescent reporter protein.

39. The expression construct of claim 33, wherein the expression construct comprises or encodes a skipping element.

40. The expression construct of claim 39, wherein the skipping element comprises a 2A peptide or an internal ribosome entry site (IRES).

41. The expression construct of claim 40, wherein the 2A peptide comprises T2A, P2A, E2A, and/or F2A.

42. The expression construct of claim 36, wherein the construct comprises the elements of CN1367, CN1244, CN1389, CN1390, CN1180, CN1203, CN1498, CN1499, CN1500, CN2001, CN2002, CN2003, CN1504 and CN1512, CN2004 and CN2005, CN2006 and CN2007, or CN2008 and CN2009.

43. The expression construct of claim 36, wherein the construct comprises SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and/or SEQ ID NO: 52.

44. The expression construct of claim 36, wherein the construct comprises SEQ ID NO: 45 and SEQ ID NO: 46, SEQ ID NO: 47 and SEQ ID NO: 48, SEQ ID NO: 49 and SEQ ID NO: 50, or SEQ ID NO: 51, and SEQ ID NO: 52

45. The expression construct of claim 36, wherein the AAV vector is associated with a capsid that crosses the blood brain barrier.

46. The expression construct of claim 45, wherein the capsid comprises PHP.Eb.

47. The expression construct of claim 45, wherein the capsid comprises SEQ ID NO: 53.

48. The expression construct of claim 45, wherein the capsid comprises AAV9 capsid with a SEQ ID NO: 54 or SEQ ID NO: 55 insert.

49. The expression construct of claim 45, wherein the capsid comprises AAV2 capsid with a SEQ ID NO: 56 insert.

50. A composition comprising the expression construct of claim 33.

51. A cell comprising the expression construct of claim 33.

52. A non-human animal comprising the expression construct of claim 33.

53. A kit comprising the expression construct of claim 33.

54. A method of rescuing voltage-gated sodium channel function in a defective cell in need thereof comprising administering a therapeutically effective amount of a composition of claim 50 to the cell.

55. A method of rescuing voltage-gated sodium channel function in a subject in need thereof comprising administering a therapeutically effective amount of a composition of claim 50 to the subject.

56. The method of claim 55, wherein the subject is need thereof due to a diagnosis of epilepsy, an SCN1A-related seizure disorder, simple febrile seizures (FS), generalized epilepsy with febrile seizures plus (GEFS+), Dravet Syndrome (DS), intractable childhood epilepsy with generalized tonic-clonic seizures (ICE-GTC), intractable infantile partial seizures, myoclonic-astatic epilepsy, Lennox-Gastaut syndrome (LGS), or infantile spasms.

57. The method of claim 55, wherein the subject is a pediatric patient.

58. The method of claim 55, wherein the subject is less than 4 years old.

59. The method of claim 55, wherein the subject is a transgenic Scn1a^{+/-} mouse undergoing a temperature-induced febrile seizure test.

60. The method of claim 55, wherein the composition is administered intravenously.

61. The method of claim 55, wherein the composition is administered intrathecally into cerebrospinal fluid, via the lateral ventricles or cisterna magna or lumbar space or cannula into the foramen magnum.

62. A vector system comprising two AAV vectors wherein the first vector encodes the N-terminal portion of a protein that rescues voltage-gated sodium channel activity in a cell in need thereof, and the second vector encodes the C-terminal portion of the protein, wherein the portion of the gene encoded by the two vectors overlaps to provide a region of homology for homologous recombination to produce the full-length Nav1.1 protein, and wherein the first vector comprises a promoter and an enhancer that consists of SEQ ID NO: 3 but does not comprise a termination signal or a polyA signal and the second vector comprises a termination signal and a polyA signal but does not comprise a promoter or an enhancer.

63. The vector system of claim 62, wherein the protein comprises human SCN1A.

64. The vector system of claim 62, wherein the region of homology is 75-1000 base pairs.

65. The vector system of claim 62, wherein the region of homology is 550-650 base pairs.

66. The vector system of claim 62, wherein the vectors selectively express the protein in inhibitory neurons.

67. The vector system of claim 62, wherein the region of homology further comprises an intron element with a splice donor site on the first vector and a splice acceptor site on the second vector, so that trans-splicing across the homologous region drives more efficient full-length protein reconstitution following recombination.

68. The vector system of claim 62, wherein the vector system comprises the elements of CN1504, CN1512, CN2004, CN2005, CN2006, CN2007, CN2008, and/or CN2009.

69. The vector system of claim 62, wherein the vector system comprises the elements of CN1504 and CN1512, CN2004 and CN2005, CN2006 and CN2007, or CN2008 and CN2009.

70. The vector system of claim 62, wherein the vector system comprises SEQ ID NO: 45 and SEQ ID NO: 46, SEQ ID NO: 47 and SEQ ID NO: 48, SEQ ID NO: 49 and SEQ ID NO: 50, or SEQ ID NO: 51, and SEQ ID NO: 52.

71. A viral vector comprising SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, or SEQ ID NO: 52.


SEQUENCE LISTING (163 sequences)

SEQ ID NO: 1 - DNA, 528 bp, synthetic construct
  misc_feature 1..528: note = hDLX I56i enhancer
  source 1..528: mol_type = other DNA
tatgcactcacagtggtttggcatgcatctggtgaattttttttaacgaaaaattagtgttggtttcgatgtatggtagcattctccctaacgtaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagg

SEQ ID NO: 2 - DNA, 131 bp, synthetic construct
  misc_feature 1..131: note = Core of the hDLX I56i enhancer
  source 1..131: mol_type = other DNA
ctaaataaagatggctttttagtattaaaagtggaagaaaattacaggtaattatctttgacggtaaaaacgctgtaatcagcgggctacatgaaaaattactctaattatggctgcatttaagagaatgg

SEQ ID NO: 3 - DNA, 393 bp, synthetic construct
  misc_feature 1..393: note = 3xhI56iCore, Triply Concatamerized Core of the hDLX I56i enhancer
  source 1..393: mol_type = other DNA
ctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatgg

SEQ ID NO: 4 - DNA, 527 bp, Mus musculus
  source 1..527: mol_type = genomic DNA
tatacactcacagtggtttggcatatatttggtgaaattttttaaggaaaaattagtgttggtttcgatatatggtagctttttctctaacataatttgaataattcagcaaagccctactaccagctgtacttctgcagcctcttccattctttccagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagg

SEQ ID NO: 5 - DNA, 281 bp, Danio rerio
  source 1..281: mol_type = genomic DNA
acattgtaattttagataatatcccaagcgttcactctcctcggcaatttgtacatgaataaccgaataatttcatcttttgtttcgtctttgccacttcaaatccaaataaagatgccttttagtattaaaagtggtagaaaattacaggtaattatctttgacggtaaaaacgctgtaatcagcgggctacatcaaaaattaccctaattatgtctgcatttatgagaatggaaaaaaaccctctcttggataaaacccataaattgtcccaaatatct

SEQ ID NO: 6 - DNA, 130 bp, Danio rerio
  source 1..130: mol_type = genomic DNA
ccaaataaagatgccttttagtattaaaagtggtagaaaattacaggtaattatctttgacggtaaaaacgctgtaatcagcgggctacatcaaaaattaccctaattatgtctgcatttatgagaatgg

SEQ ID NO: 7 - DNA, 390 bp, Danio rerio
  source 1..390: mol_type = genomic DNA
ccaaataaagatgccttttagtattaaaagtggtagaaaattacaggtaattatctttgacggtaaaaacgctgtaatcagcgggctacatcaaaaattaccctaattatgtctgcatttatgagaatggccaaataaagatgccttttagtattaaaagtggtagaaaattacaggtaattatctttgacggtaaaaacgctgtaatcagcgggctacatcaaaaattaccctaattatgtctgcatttatgagaatggccaaataaagatgccttttagtattaaaagtggtagaaaattacaggtaattatctttgacggtaaaaacgctgtaatcagcgggctacatcaaaaattaccctaattatgtctgcatttatgagaatgg

SEQ ID NO: 8 - DNA, 376 bp, synthetic construct
  misc_feature 1..376: note = hDLX I12b enhancer
  source 1..376: mol_type = other DNA
cagctgcaaacccaagagggtcagcatcatttcactgtattctcttcttgattacaagccgggcccatcaaacacaacataattacagtaatttcaggtttatttattctaatgcagtttccccatctctctggtaattatgagcaattttttcgcccagggaatctttttgcattaacaaaagagataacgcactgaaagccaaatttgctgtgcattgagaaaaggaaaaaaaaaaatcaaataggtgcgagctgccatctctgcaattctctggtaccggagccggcaaattgcttgcaggtgtatggagcaagcttgtcaatggccaggcctccaaattagcaaatgcacagcagcaaagtaatgaagacag

SEQ ID NO: 9 - DNA, 984 bp, synthetic construct
  misc_feature 1..984: note = NavSheP-D60N, codon optimized, with N-terminal 3x HA tag
  source 1..984: mol_type = other DNA
atggtttacccgtatgatgtcccggattacgctggcagctacccatacgatgtacccgactatgccggcagttatccctacgacgtccctgactacgcatctacgtcccttttgaatgcgcctaccggccttcaagctagagtcattaatctcgtcgaacaaaactggtttggacactttatactgactctcatactcattaatgctgtgcagcttggaatggaaactagcgccagcctcatggcacaatatggcgcgctgcttatgtccttgaataaggtccttctctctgtgttcgtggtcgaactgctgctccggatttatgcgtatcggggcaagttttttaaggacccgtggaatgtgtttgacttcactgttattgttattgctctgattcctgcatctggcccattggctgtcctccgctccctccgagttctccgcgtcttgagggttctgacgattgtccccagcatgaaaagagtagtgtcagcactgcttgggagcttgcccgggttggcctccattgcaaccgtgcttctgttgatctattacgttttcgctgtgatcgccactaaaattttcggggatgcttttccggaatggttcgggacgatagcggactccttctataccctttttcaaattatgaccttggaaagttggtctatggggatctctaggccagtgatggaggtgtacccttacgcttgggtattctttgtgccctttattcttgttgctacttttaccatgcttaaccttttcatcgccatcatagtgaatactatgcagacattctctgacgaggaacatgctctggagcgagagcaagataaacagatcttggaacaggagcagagacaaatgcacgaggaactgaaggccattcgactcgagcttcagcaactccaaacccttttgcgaaatgcggctggggactcctccaatgtctccacaaagggcaatatcggctcagactaa

SEQ ID NO: 10 - DNA, 888 bp, synthetic construct
  misc_feature 1..888: note = NavSheP endogenous sequence
  source 1..888: mol_type = other DNA
atgagtacatctttacttaacgcgccaacgggtttgcaggcacgagtgattaacttggttgagcaaaactggtttggtcattttattttgacattgattttaatcaacgcggtgcagttaggtatggagacctcagccagcctgatggcgcaatacggtgctttgttgatgagtcttgataaggtgctgctgagtgtatttgtggtggagttattgctgcggatttatgcctacagggggaaattttttaaagacccttggaacgtgttcgattttaccgtgatagtgatagcactgatccctgcatctgggccattggctgtcctgcgttcgctcagggtattgcgggtgctgagagtgttaacaattgtgccatcaatgaaacgggtggtgtctgcgctgttgggatcacttcctggattggcatcgatcgccacagtattactgctgatttattatgtgtttgcggtgatcgctaccaaaatttttggcgatgcattccctgaatggtttggcactattgctgactcattttataccctatttcaaataatgacgcttgaaagctggtctatgggaatttcgcggccagtgatggaagtctacccttatgcttgggtatttttcgtaccatttattctggtagcgactttcacaatgctaaatttgtttattgcgattatcgtcaataccatgcaaaccttcagcgacgaagagcatgcattagagcgtgagcaagacaaacaaatcttagagcaggaacaaagacaaatgcacgaggagttgaaagccatcagactcgagctacaacaattacaaaccttgctgcgcaatgctgctggtgattcttctaatgtgtcgacaaagggaaacattggttctgactaa

SEQ ID NO: 11 - DNA, 855 bp, synthetic construct
  misc_feature 1..855: note = NavBp, endogenous sequence
  source 1..855: mol_type = other DNA
atggaaaacaatccagccgaacaacaagttccaccattagtagccttagctcagcgtatcgtctttcataaggcctttaccccaactattattaccttgattatcattaatgccattattgtaggccttgaaacatatcctactgtttatcaaggttataatgattggttctacgcagcagatttagccttactttggatttttacaattgagattacactgcgttttatcgcagcgagaccgactaaatctttttttaaaagcagctggaactggtttgatttattaatcgttcttgccggtcatgtctttgccggtgctcattttgtaacggttcttcgtatcctgcgcgttcttcgcgtattacgtgccatttctgtcattccttctctgcgtcgtttagtcgatgctttgctgatgaccatcccggctttaggaaacattatgatcctgatgggaattattttctatattttcgctgtgattggaacgatgttatttgcttctgtagcacctgagtactttggtaacttacagctttctttattaacattattccaagttgttacacttgaatcttgggcaagcggtgtcatgaggccgatttttgcagaggtttggtggtcttggatttattttgtcatctttattttagtagggacatttattgtctttaacttatttatcggtgttatcgttaataacgttgaaaaagcaaacgaagaagaactcaaatcagaattagatgataaagaggcagatacaaaagaagagcttgcttctctgcgtaatgaagtagcagagatgaaagacctcattaaacaaatgcataaacagcaaacaaaaaaagggtaa

SEQ ID NO: 12 - DNA, 951 bp, synthetic construct
  misc_feature 1..951: note = NavBp, codon optimized, with N-terminal 3x HA tag
  source 1..951: mol_type = other DNA
atggtttacccgtatgatgtcccggattacgctggcagctacccatacgatgtacccgactatgccggcagttatccctacgacgtccctgactacgcagaaaacaacccagccgaacagcaagtcccacccctcgtggcgctcgcccaacgcatagtatttcacaaggcgtttacgccgacgataatcaccctcatcattattaatgcgatcattgtgggactcgagacatacccaacggtttaccagggttacaatgattggttctatgctgccgaccttgctttgttgtggatattcactattgaaatcacgctccgattcatcgccgcccgaccgacgaagagtttcttcaagtctagctggaactggtttgatctgcttatcgtattggcgggccacgtcttcgctggcgcccattttgttacggtgcttaggatcctccgcgtcctgagggtcctcagagctatctcagtcatacccagtctccggcggctggttgacgcacttttgatgacaatcccagcactcggtaacatcatgatactgatggggattattttttacatattcgcggttatcgggacgatgctctttgcatcagtagcgccagaatactttggcaatttgcagctgtctctgcttacactgttccaagtggttacgctggaaagttgggctagtggggttatgcgacctatttttgccgaagtctggtggtcttggatctattttgtaatctttattctcgtgggaactttcatagtatttaaccttttcattggcgtcatcgtgaacaatgtggaaaaagctaacgaagaggaactgaaaagcgaactggatgataaagaggctgatacaaaagaagaactggcatcattgcgaaacgaggtggcagaaatgaaggatctcataaaacagatgcataaacagcaaacaaaaaagggttaa

SEQ ID NO: 13 - DNA, 825 bp, synthetic construct
  misc_feature 1..825: note = NavMs, endogenous sequence
  source 1..825: mol_type = other DNA
atgtcacgcaaaataagagatttaatcgaatccaaacgctttcaaaacgtcatcaccgccattattgtgctcaatggcgctgtgctgggtctgctgaccgatacaaccctatcggcctccagccaaaacctgctggagcgtgtggatcaactttgtctgactatctttattgttgaaatatccctgaaaatatacgcctatggcgtgcgaggctttttccgcagcggctggaatctgtttgattttgtgattgtggccatcgcgcttatgcccgcccagggtagcctatcggtgctgcgaaccttccgtatattccgcgtcatgcggctcgtatcggtcataccaaccatgcgaagagtggtgcaaggcatgctcttggcactgcccggcgtgggatcggtagcggcactgttgacggtggtcttctatattgcggctgtcatggccaccaatctctacggggcaaccttccctgaatggtttggtgatcttagcaagagcctgtacacactatttcaggtgatgaccttagagtcatggtctatgggcattgtgcgtccagtgatgaacgttcatcccaacgcatgggtttttttcatccccttcatcatgctcaccacctttaccgtgctcaacctgtttattggcattattgtagatgccatggccatcaccaaggaacaggaggaagaggccaaaaccggccaccaccaagagcctattagccaaacattgctccatctgggagatcgcctagataggatcgaaaagcagcttgcgcaaaacaacgagctcttacaacgacaacagccgcaaaaaaaatag

SEQ ID NO: 14 - DNA, 954 bp, synthetic construct
  misc_feature 1..954: note = NavMs, codon optimized, with N-terminal 3x HA tag and linker
  source 1..954: mol_type = other DNA
atggtttatccgtatgatgttcctgactatgcaggatcctatccttatgatgttcccgattacgctggttcttacccttacgatgttcccgattatgccagttctggattggtgccacgaggcagccacatgagccggaagatcagagatcttatcgaatctaagagatttcagaatgttattaccgcgataatcgtactcaacggggcggtgctcggtctcctcaccgataccacattgagcgcttctagccagaacctgctcgaaagggttgaccaactgtgcctgacaatttttatcgtggaaattagcttgaaaatttacgcctacggcgttcgcggttttttccggagcggttggaatctttttgacttcgttatcgttgccatcgcgctcatgcccgcacagggttctttgtctgtgttgaggacattccgaatatttcgcgtgatgcgcttggtatccgtgatccctacgatgcgccgcgtcgtacaaggaatgttgctggctctccccggcgtcgggagcgttgctgccctccttaccgtggtattttacatagcggcggttatggctactaatctttacggagctaccttcccggagtggttcggggatttgtccaagagcctctatacattgtttcaagttatgaccctggagtcctggtctatgggcattgtccggcccgtaatgaacgtacacccaaatgcgtgggtgtttttcattccattcatcatgctgactacctttaccgtgctgaacttgttcattgggattatcgtggatgcgatggccatcactaaggagcaagaagaagaggctaaaactggccaccaccaagagccaatttctcaaaccctcttgcatctcggggaccgactggaccgcattgagaagcaactcgcgcagaacaatgagctgttgcagcgacagcaacctcaaaaaaaataa

SEQ ID NO: 15 - DNA, 885 bp, synthetic construct
  misc_feature 1..885: note = NavMs, codon optimized, with N-terminal His tag and linker
  source 1..885: mol_type = other DNA
atgggcagcagccatcatcatcatcatcacagcagcggcctggtgccgcgcggcagccatatgtcacgcaaaatccgcgatttaatcgaatccaaacgctttcaaaacgtcatcaccgccattattgtgctcaatggcgctgtgctgggtctgctgaccgatacaaccctgtcggcctccagccaaaacctgctggagcgtgtggatcaactttgtctgactatctttattgttgaaatctccctgaaaatctacgcctatggcgtgcgcggctttttccgcagcggctggaatctgtttgattttgtgattgtggccatcgcgcttatgccggcccagggtagcctgtcggtgctgcgtaccttccgtatcttccgcgtcatgcgcctcgtatcggtcatcccaaccatgcgccgtgtggtgcaaggcatgctcttggcactgccgggcgtgggctcggtagcggcactgttgacggtggtcttctatattgcggctgtcatggccaccaatctctacggggcaaccttccctgaatggtttggtgatcttagcaagagcctgtacacactgtttcaggtgatgaccttagagtcatggtctatgggcattgtgcgtccagtgatgaacgttcatccgaacgcatgggtttttttcatcccgttcatcatgctcaccacctttaccgtgctcaacctgtttattggcattattgtagatgcaatggcaatcaccaaggaacaggaggaagaggccaaaaccggtcaccatcaagaacctatttctcaaactcttcttcatcttggtgatcgtcttgatcgtattgaaaaacaacttgctcaaaataatgaacttcttcaacgtcaacaacctcaaaaaaaataa

SEQ ID NO: 16 - DNA, 6027 bp, Homo sapiens
  source 1..6027: mol_type = genomic DNA
atggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataa

SEQ ID NO: 17 - DNA, 720 bp, synthetic construct
  misc_feature 1..720: note = SYFP2
  source 1..720: mol_type = other DNA
atggtcagcaagggcgaggagctgttcaccggggtggtgcccatcctggtcgagctggacggcgacgtcaatggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaataa

SEQ ID NO: 18 - DNA, 78 bp, synthetic construct
  misc_feature 1..78: note = P2A Encoding Sequence
  source 1..78: mol_type = other DNA
ggcagcggcgccaccaacttcagcctgctgaagcaggccggcgacgtggaggagaaccccggccccggagctagcgga

SEQ ID NO: 19 - DNA, 246 bp, synthetic construct
  misc_feature 1..246: note = WPRE3
  source 1..246: mol_type = other DNA
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgg

SEQ ID NO: 20 - DNA, 204 bp, synthetic construct
  misc_feature 1..204: note = BGHpA
  source 1..204: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 21 - protein, 33 aa, synthetic construct
  REGION 1..33: note = N-terminal 3XHA tag (Protein)
MVYPYDVPDYAGSYPYDVPDYAGSYPYDVPDYA

SEQ ID NO: 22 - DNA, 99 bp, synthetic construct
  misc_feature 1..99: note = N-terminal 3XHA tag
  source 1..99: mol_type = other DNA
atggtttacccgtatgatgtcccggattacgctggcagctacccatacgatgtacccgactatgccggcagttatccctacgacgtccctgactacgca

SEQ ID NO: 23 - DNA, 3133 bp, synthetic construct
  misc_feature 1..3133: note = hSCN1A N-term of two-part expression system
  source 1..3133: mol_type = other DNA
atggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattt

SEQ ID NO: 24 - DNA, 3639 bp, synthetic construct
  misc_feature 1..3639: note = hSCN1A C-term of two-part expression system with c-terminal 3XHA sequence
  source 1..3639: mol_type = other DNA
ctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaaggaggtggtggttcaggtgggggcggctcagagtacccctatgatgtccctgattatgcggcggaatacccctatgacgtgccggactacgcggctgaatatccgtatgacgttcccgattatgcggctaagctcgaataatga

SEQ ID NO: 25 - DNA, 604 bp, synthetic construct
  misc_feature 1..604: note = 604 bp homology region of hSCN1A N term and C term that can be used in two-part expression system
  source 1..604: mol_type = other DNA
ctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattt

SEQ ID NO: 26 - protein, 26 aa, synthetic construct
  REGION 1..26: note = P2A Translation from CN1498
GSGATNFSLLKQAGDVEENPGPGASG

SEQ ID NO: 27 - protein, 21 aa, synthetic construct
  REGION 1..21: note = T2A
GSGEGRGSLLTCGDVEENPGP

SEQ ID NO: 28 - protein, 24 aa, synthetic construct
  REGION 1..24: note = E2A
GSGQCTNYALLKLAGDVESNPGPP

SEQ ID NO: 29 - protein, 25 aa, synthetic construct
  REGION 1..25: note = F2A
GSGVKQTLNFDLLKLAGDVESNPGP

SEQ ID NO: 30 - DNA, 53 bp, synthetic construct
  misc_feature 1..53: note = MinBglobin
  source 1..53: mol_type = other DNA
gggctgggcataaaagtcagggcagagccatctattgcttacatttgcttctg

SEQ ID NO: 31 - DNA, 68 bp, synthetic construct
  misc_feature 1..68: note = minCMV
  source 1..68: mol_type = other DNA
gaggtaggcgtgtacggtgggaggcctatataagcagagctcgtttagtgaaccgtcagatcgcctgg

SEQ ID NO: 32 - protein, 11 aa, synthetic construct
  REGION 1..11: note = PHP.eB capsid: Corresponds to the AAV9 VP1 capsid sequence with a modification that starts at amino acid residue 586 where S AQ A are changed to S DGTLAVPFK A.
SDGTLAVPFKA

SEQ ID NO: 33 - DNA, 2843 bp, synthetic construct
  misc_feature 1..2843: note = CN1367 - The portion between L-ITR and R-ITR: positions 142-2984
  source 1..2843: mol_type = other DNA
gcggccgcacgcgtataggtaccgagctctatgcactcacagtggtttggcatgcatctggtgaattttttttaacgaaaaattagtgttggtttcgatgtatggtagcattctccctaacgtaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagggagctcgggctgggcataaaagtcagggcagagccatctattgcttacatttgcttctgggatccagatctttcgaagctagcgctaccggtcgccaccatgggcagcagccatcatcatcatcatcacagcagcggcctggtgccgcgcggcagccatatgtcacgcaaaatccgcgatttaatcgaatccaaacgctttcaaaacgtcatcaccgccattattgtgctcaatggcgctgtgctgggtctgctgaccgatacaaccctgtcggcctccagccaaaacctgctggagcgtgtggatcaactttgtctgactatctttattgttgaaatctccctgaaaatctacgcctatggcgtgcgcggctttttccgcagcggctggaatctgtttgattttgtgattgtggccatcgcgcttatgccggcccagggtagcctgtcggtgctgcgtaccttccgtatcttccgcgtcatgcgcctcgtatcggtcatcccaaccatgcgccgtgtggtgcaaggcatgctcttggcactgccgggcgtgggctcggtagcggcactgttgacggtggtcttctatattgcggctgtcatggccaccaatctctacggggcaaccttccctgaatggtttggtgatcttagcaagagcctgtacacactgtttcaggtgatgaccttagagtcatggtctatgggcattgtgcgtccagtgatgaacgttcatccgaacgcatgggtttttttcatcccgttcatcatgctcaccacctttaccgtgctcaacctgtttattggcattattgtagatgcaatggcaatcaccaaggaacaggaggaagaggccaaaaccggtcaccatcaagaacctatttctcaaactcttcttcatcttggtgatcgtcttgatcgtattgaaaaacaacttgctcaaaataatgaacttcttcaacgtcaacaacctcaaaaaaaaggcagcggcgccaccaacttcagcctgctgaagcaggccggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 34 - DNA, 2835 bp, synthetic construct
  misc_feature 1..2835: note = CN1500 - The portion between L-ITR and R-ITR: positions 142-2976
  source 1..2835: mol_type = other DNA
gcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctggtcgacacaattggaggtaggcgtgtacggtgggaggcctatataagcagagctcgtttagtgaaccgtcagatcgcctggaggatccttcgaaaagcttgctaccggtcgccaccatggtcagcaagggcgaggagctgttcaccggggtggtgcccatcctggtcgagctggacggcgacgtcaatggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaaggcagcggcgccaccaacttcagcctgctgaagcaggccggcgacgtggaggagaaccccggccccggagctagcggaatggtttacccgtatgatgtcccggattacgctggcagctacccatacgatgtacccgactatgccggcagttatccctacgacgtccctgactacgcatctacgtcccttttgaatgcgcctaccggccttcaagctagagtcattaatctcgtcgaacaaaactggtttggacactttatactgactctcatactcattaatgctgtgcagcttggaatggaaactagcgccagcctcatggcacaatatggcgcgctgcttatgtccttgaataaggtccttctctctgtgttcgtggtcgaactgctgctccggatttatgcgtatcggggcaagttttttaaggacccgtggaatgtgtttgacttcactgttattgttattgctctgattcctgcatctggcccattggctgtcctccgctccctccgagttctccgcgtcttgagggttctgacgattgtccccagcatgaaaagagtagtgtcagcactgcttgggagcttgcccgggttggcctccattgcaaccgtgcttctgttgatctattacgttttcgctgtgatcgccactaaaattttcggggatgcttttccggaatggttcgggacgatagcggactccttctataccctttttcaaattatgaccttggaaagttggtctatggggatctctaggccagtgatggaggtgtacccttacgcttgggtattctttgtgccctttattcttgttgctacttttaccatgcttaaccttttcatcgccatcatagtgaatactatgcagacattctctgacgaggaacatgctctggagcgagagcaagataaacagatcttggaacaggagcagagacaaatgcacgaggaactgaaggccattcgactcgagcttcagcaactccaaacccttttgcgaaatgcggctggggactcctccaatgtctccacaaagggcaatatcggctcagactaatgaccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 35 - DNA, 5681 bp, synthetic construct
  misc_feature 1..5681: note = CN1498 - The portion between L-ITR and R-ITR: positions 142-2943
  source 1..5681: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctggtcgacacaattggaggtaggcgtgtacggtgggaggcctatataagcagagctcgtttagtgaaccgtcagatcgcctggaggatccttcgaaaagcttgctaccggtcgccaccatggtcagcaagggcgaggagctgttcaccggggtggtgcccatcctggtcgagctggacggcgacgtcaatggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaaggcagcggcgccaccaacttcagcctgctgaagcaggccggcgacgtggaggagaaccccggccccggagctagcggaatggtttacccgtatgatgtcccggattacgctggcagctacccatacgatgtacccgactatgccggcagttatccctacgacgtccctgactacgcagaaaacaacccagccgaacagcaagtcccacccctcgtggcgctcgcccaacgcatagtatttcacaaggcgtttacgccgacgataatcaccctcatcattattaatgcgatcattgtgggactcgagacatacccaacggtttaccagggttacaatgattggttctatgctgccgaccttgctttgttgtggatattcactattgaaatcacgctccgattcatcgccgcccgaccgacgaagagtttcttcaagtctagctggaactggtttgatctgcttatcgtattggcgggccacgtcttcgctggcgcccattttgttacggtgcttaggatcctccgcgtcctgagggtcctcagagctatctcagtcatacccagtctccggcggctggttgacgcacttttgatgacaatcccagcactcggtaacatcatgatactgatggggattattttttacatattcgcggttatcgggacgatgctctttgcatcagtagcgccagaatactttggcaatttgcagctgtctctgcttacactgttccaagtggttacgctggaaagttgggctagtggggttatgcgacctatttttgccgaagtctggtggtcttggatctattttgtaatctttattctcgtgggaactttcatagtatttaaccttttcattggcgtcatcgtgaacaatgtggaaaaagctaacgaagaggaactgaaaagcgaactggatgataaagaggctgatacaaaagaagaactggcatcattgcgaaacgaggtggcagaaatgaaggatctcataaaacagatgcataaacagcaaacaaaaaagggttaatgaccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 36 - DNA, 5684 bp, synthetic construct
  misc_feature 1..5684: note = CN1499 - The portion between L-ITR and R-ITR: positions 142-2946
  source 1..5684: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctggtcgacacaattggaggtaggcgtgtacggtgggaggcctatataagcagagctcgtttagtgaaccgtcagatcgcctggaggatccttcgaaaagcttgctaccggtcgccaccatggtcagcaagggcgaggagctgttcaccggggtggtgcccatcctggtcgagctggacggcgacgtcaatggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaaggcagcggcgccaccaacttcagcctgctgaagcaggccggcgacgtggaggagaaccccggccccggagctagcggaatggtttatccgtatgatgttcctgactatgcaggatcctatccttatgatgttcccgattacgctggttcttacccttacgatgttcccgattatgccagttctggattggtgccacgaggcagccacatgagccggaagatcagagatcttatcgaatctaagagatttcagaatgttattaccgcgataatcgtactcaacggggcggtgctcggtctcctcaccgataccacattgagcgcttctagccagaacctgctcgaaagggttgaccaactgtgcctgacaatttttatcgtggaaattagcttgaaaatttacgcctacggcgttcgcggttttttccggagcggttggaatctttttgacttcgttatcgttgccatcgcgctcatgcccgcacagggttctttgtctgtgttgaggacattccgaatatttcgcgtgatgcgcttggtatccgtgatccctacgatgcgccgcgtcgtacaaggaatgttgctggctctccccggcgtcgggagcgttgctgccctccttaccgtggtattttacatagcggcggttatggctactaatctttacggagctaccttcccggagtggttcggggatttgtccaagagcctctatacattgtttcaagttatgaccctggagtcctggtctatgggcattgtccggcccgtaatgaacgtacacccaaatgcgtgggtgtttttcattccattcatcatgctgactacctttaccgtgctgaacttgttcattgggattatcgtggatgcgatggccatcactaaggagcaagaagaagaggctaaaactggccaccaccaagagccaatttctcaaaccctcttgcatctcggggaccgactggaccgcattgagaagcaactcgcgcagaacaatgagctgttgcagcgacagcaacctcaaaaaaaataatgaccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 37 - DNA, 4780 bp, synthetic construct
  misc_feature 1..4780: note = CN1244 - The portion between L-ITR and R-ITR: positions 142-2042
  source 1..4780: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataggtaccgagctctatgcactcacagtggtttggcatgcatctggtgaattttttttaacgaaaaattagtgttggtttcgatgtatggtagcattctccctaacgtaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 38 - DNA, 4398 bp, synthetic construct
  misc_feature 1..4398: note = CN1389 - The portion between L-ITR and R-ITR corresponds to positions 142-1897
  source 1..4398: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggacctgcagggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 39 - DNA, 4635 bp, synthetic construct
  misc_feature 1..4635: note = CN1390 - The portion between L-ITR and R-ITR corresponds to positions 142-1660
  source 1..4635: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 40 - DNA, 4841 bp, synthetic construct
  misc_feature 1..4841: note = CN1203 - The portion between L-ITR and R-ITR corresponds to positions 183-2052
  source 1..4841: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggacgcgtataggtaccgagctctatgcactcacagtggtttggcatgcatctggtgaattttttttaacgaaaaattagtgttggtttcgatgtatggtagcattctccctaacgtaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtagggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 41 - DNA, 4680 bp, synthetic construct
  misc_feature 1..4680: note = CN1180 - The portion between L-ITR and R-ITR corresponds to positions 183-1891
  source 1..4680: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggacgcgtcagctgcaaacccaagagggtcagcatcatttcactgtattctcttcttgattacaagccgggcccatcaaacacaacataattacagtaatttcaggtttatttattctaatgcagtttccccatctctctggtaattatgagcaattttttcgcccagggaatctttttgcattaacaaaagagataacgcactgaaagccaaatttgctgtgcattgagaaaaggaaaaaaaaaaatcaaataggtgcgagctgccatctctgcaattctctggtaccggagccggcaaattgcttgcaggtgtatggagcaagcttgtcaatggccaggcctccaaattagcaaatgcacagcagcaaagtaatgaagacaggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgactagtgcatgcccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 42 - DNA, 4761 bp, synthetic construct
  misc_feature 1..4761: note = CN2001 - The portion between L-ITR and R-ITR corresponds to positions 142-2023
  source 1..4761: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaccaccatggaaaacaacccagccgaacagcaagtcccacccctcgtggcgctcgcccaacgcatagtatttcacaaggcgtttacgccgacgataatcaccctcatcattattaatgcgatcattgtgggactcgagacatacccaacggtttaccagggttacaatgattggttctatgctgccgaccttgctttgttgtggatattcactattgaaatcacgctccgattcatcgccgcccgaccgacgaagagtttcttcaagtctagctggaactggtttgatctgcttatcgtattggcgggccacgtcttcgctggcgcccattttgttacggtgcttaggatcctccgcgtcctgagggtcctcagagctatctcagtcatacccagtctccggcggctggttgacgcacttttgatgacaatcccagcactcggtaacatcatgatactgatggggattattttttacatattcgcggttatcgggacgatgctctttgcatcagtagcgccagaatactttggcaatttgcagctgtctctgcttacactgttccaagtggttacgctggaaagttgggctagtggggttatgcgacctatttttgccgaagtctggtggtcttggatctattttgtaatctttattctcgtgggaactttcatagtatttaaccttttcattggcgtcatcgtgaacaatgtggaaaaagctaacgaagaggaactgaaaagcgaactggatgataaagaggctgatacaaaagaagaactggcatcattgcgaaacgaggtggcagaaatgaaggatctcataaaacagatgcataaacagcaaacaaaaaagggttaatgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 43 - DNA, 4732 bp, synthetic construct
  misc_feature 1..4732: note = CN2002 - The portion between L-ITR and R-ITR corresponds to positions 142-1993
  source 1..4732: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaccaccatgagccggaagatcagagatcttatcgaatctaagagatttcagaatgttattaccgcgataatcgtactcaacggggcggtgctcggtctcctcaccgataccacattgagcgcttctagccagaacctgctcgaaagggttgaccaactgtgcctgacaatttttatcgtggaaattagcttgaaaatttacgcctacggcgttcgcggttttttccggagcggttggaatctttttgacttcgttatcgttgccatcgcgctcatgcccgcacagggttctttgtctgtgttgaggacattccgaatatttcgcgtgatgcgcttggtatccgtgatccctacgatgcgccgcgtcgtacaaggaatgttgctggctctccccggcgtcgggagcgttgctgccctccttaccgtggtattttacatagcggcggttatggctactaatctttacggagctaccttcccggagtggttcggggatttgtccaagagcctctatacattgtttcaagttatgaccctggagtcctggtctatgggcattgtccggcccgtaatgaacgtacacccaaatgcgtgggtgtttttcattccattcatcatgctgactacctttaccgtgctgaacttgttcattgggattatcgtggatgcgatggccatcactaaggagcaagaagaagaggctaaaactggccaccaccaagagccaatttctcaaaccctcttgcatctcggggaccgactggaccgcattgagaagcaactcgcgcagaacaatgagctgttgcagcgacagcaacctcaaaaaaaataatgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgts

SEQ ID NO: 44 - DNA, 4794 bp, synthetic construct
  misc_feature 1..4794: note = CN2003 - The portion between L-ITR and R-ITR corresponds to positions 142-2056
  source 1..4794: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaccaccatgtctacgtcccttttgaatgcgcctaccggccttcaagctagagtcattaatctcgtcgaacaaaactggtttggacactttatactgactctcatactcattaatgctgtgcagcttggaatggaaactagcgccagcctcatggcacaatatggcgcgctgcttatgtccttgaataaggtccttctctctgtgttcgtggtcgaactgctgctccggatttatgcgtatcggggcaagttttttaaggacccgtggaatgtgtttgacttcactgttattgttattgctctgattcctgcatctggcccattggctgtcctccgctccctccgagttctccgcgtcttgagggttctgacgattgtccccagcatgaaaagagtagtgtcagcactgcttgggagcttgcccgggttggcctccattgcaaccgtgcttctgttgatctattacgttttcgctgtgatcgccactaaaattttcggggatgcttttccggaatggttcgggacgatagcggactccttctataccctttttcaaattatgaccttggaaagttggtctatggggatctctaggccagtgatggaggtgtacccttacgcttgggtattctttgtgccctttattcttgttgctacttttaccatgcttaaccttttcatcgccatcatagtgaatactatgcagacattctctgacgaggaacatgctctggagcgagagcaagataaacagatcttggaacaggagcagagacaaatgcacgaggaactgaaggccattcgactcgagcttcagcaactccaaacccttttgcgaaatgcggctggggactcctccaatgtctccacaaagggcaatatcggctcagactaatgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 45 - DNA, 7368 bp, synthetic construct
  misc_feature 1..7368: note = CN1504 - The portion between L-ITR and R-ITR corresponds to positions 142-4489
  source 1..7368: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctggtcgacacaattggaggtaggcgtgtacggtgggaggcctatataagcagagctcgtttagtgaaccgtcagatcgcctggaggatccttcgaaaagcttgctaccggtgccaccatggtcagcaagggcgaggagctgttcaccggggtggtgcccatcctggtcgagctggacggcgacgtcaatggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaaggcagcggcgccaccaacttcagcctgctgaagcaggccggcgacgtggaggagaaccccggccccggaactagtggtatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 46 - DNA, 7044 bp, synthetic construct
  misc_feature 1..7044: note = CN1512 - The portion between L-ITR and R-ITR corresponds to positions 142-4165
  source 1..7044: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataggtaccctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaaggaggtggtggttcaggtgggggcggctcagagtacccctatgatgtccctgattatgcggcggaatacccctatgacgtgccggactacgcggctgaatatccgtatgacgttcccgattatgcggctaagctcgaataatgatgagaattcatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 47 - DNA, 6530 bp, synthetic construct
  misc_feature 1..6530: note = CN2004 - The portion between L-ITR and R-ITR corresponds to positions 142-3792
  source 1..6530: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagatctgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 48 - DNA, 6898 bp, synthetic construct
  misc_feature 1..6898: note = CN2005 - The portion between L-ITR and R-ITR corresponds to positions 142-4160
  source 1..6898: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataggtaccctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataatgacatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 49 - DNA, 7528 bp, synthetic construct
  misc_feature 1..7528: note = CN2006 - The portion between L-ITR and R-ITR corresponds to positions 142-4790
  source 1..7528: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctggtcgacacaattggaggtaggcgtgtacggtgggaggcctatataagcagagctcgtttagtgaaccgtcagatcgcctggaggatccttcgaaaagcttgctaccggtgccaccatggtcagcaagggcgaggagctgttcaccggggtggtgcccatcctggtcgagctggacggcgacgtcaatggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaagacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaaaggcagcggcgccaccaacttcagcctgctgaagcaggccggcgacgtggaggagaaccccggccccggaactagtggtatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcgtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctactcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 50 - DNA, 7409 bp, synthetic construct
  misc_feature 1..7409: note = CN2007 - The portion between L-ITR and R-ITR corresponds to positions 142-4671
  source 1..7409: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaaggaggtggtggttcaggtgggggcggctcagagtacccctatgatgtccctgattatgcggcggaatacccctatgacgtgccggactacgcggctgaatatccgtatgacgttcccgattatgcggctaagctcgaataatgatgagaattcatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 51 - DNA, 6733 bp, synthetic construct
  misc_feature 1..6733: note = CN2008 - The portion between L-ITR and R-ITR corresponds to positions 142-3995
  source 1..6733: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctggtcgacacaattggaggtaggcgtgtacggtgggaggcctatataagcagagctcgtttagtgaaccgtcagatcgcctggaggatccttcgaaaagcttgctaccggtgccaccatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcgtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctactcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 52 - DNA, 7263 bp, synthetic construct
  misc_feature 1..7263: note = CN2009 - The portion between L-ITR and R-ITR corresponds to positions 142-4525
  source 1..7263: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataatgacatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 53 - protein, 7 aa, synthetic construct
  REGION 1..7: note = AAV-BR1
NRGTEWD

SEQ ID NO: 54 - protein, 7 aa, synthetic construct
  REGION 1..7: note = AAV-PHP.S
QAVRTSL

SEQ ID NO: 55 - protein, 7 aa, synthetic construct
  REGION 1..7: note = AAV-PHP.B
TLAVPFK

SEQ ID NO: 56 - protein, 7 aa, synthetic construct
  REGION 1..7: note = AAV-PPS
DSPAHPS

SEQ ID NO: 57 - protein, 736 aa, synthetic construct
  REGION 1..736: note = Capsid protein VP1 from Adeno-associated virus 9 (AVV9)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 58 - DNA, 5799 bp, synthetic construct
  misc_feature 1..5799: note = CN2027-rAAV-3xhI56i(core)-minBG-hSCN1A_Fragment2-WP RE3-BGHpA
  source 1..5799: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaccaccatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtgttggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggtgatgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 59 - DNA, 5202 bp, synthetic construct
  misc_feature 1..5202: note = CN2027-rAAV-3xhI56i(core)-minBG-hSCN1A_Fragment2-WP RE3-BGHpA
  source 1..5202: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaccaccatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggtcgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgttgatgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 60 - DNA, 5223 bp, synthetic construct
  misc_feature 1..5223: note = CN2028-rAAV-3xhI56i(core)-minBG-hSCN1A_Fragment3-WP RE3-BGHpA
  source 1..5223: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaccaccatgtccaatcatacaacagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatcttttgatgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 61 - DNA, 5439 bp, synthetic construct
  misc_feature 1..5439: note = CN2029-rAAV-3xhI56i(core)-minBG-hSCN1A_Fragment4-WP RE3-BGHpA
  source 1..5439: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccagatctttcgaagctagcgctaccaccatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataatgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 62 - protein, 630 aa, synthetic construct
  REGION 1..630: note = hSCN1A_Fragment1_Protein Sequence
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSR

SEQ ID NO: 63 - protein, 431 aa, synthetic construct
  REGION 1..431: note = hSCN1A_Fragment2_Protein Sequence
MLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSC

SEQ ID NO: 64 - protein, 438 aa, synthetic construct
  REGION 1..438: note = hSCN1A_Fragment3_Protein Sequence
MSNHTTEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIF

SEQ ID NO: 65 - protein, 510 aa, synthetic construct
  REGION 1..510: note = hSCN1A_Fragment4_Protein Sequence
MTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 66 - DNA, 13 bp, synthetic construct
  misc_feature 1..13: note = synthetic oligonucleotide for upregulation of SCN1A expression
  source 1..13: mol_type = other DNA
tcgactttgaaaa

SEQ ID NO: 67 - DNA, 21 bp, synthetic construct
  misc_feature 1..21: note = synthetic oligonucleotide for upregulation of SCN1A expression
  source 1..21: mol_type = other DNA
cctctccacgcgcagtacatt

SEQ ID NO: 68 - DNA, 20 bp, synthetic construct
  misc_feature 1..20: note = synthetic oligonucleotide for upregulation of SCN1A expression
  modified_base 1..20: mod_base = OTHER; note = phosphorothioated base
  modified_base 1..20: mod_base = OTHER; note = phosphorothioated bases
  source 1..20: mol_type = other DNA
tcggtgtccactctggcagt

SEQ ID NO: 69 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
tgcactgtgggagcctgtct

SEQ ID NO: 70 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
gtagcactgtggacatcggc

SEQ ID NO: 71 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
gtagaagaacagcccgtagtg

SEQ ID NO: 72 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
gtggtctctgcattctgtca

SEQ ID NO: 73 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
gtggtataggaactggcagca

SEQ ID NO: 74 - DNA, 19 bp, synthetic construct
  misc_feature 1..19: note = synthetic oligonucleotide for upregulation of SCN1A expression
  modified_base 1..18: mod_base = OTHER; note = phosphorothioated bases
  source 1..19: mol_type = other DNA
gtccaatcatacagcagaa

SEQ ID NO: 75 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
gtgactgtaccaattgctgt

SEQ ID NO: 76 - DNA, 19 bp, synthetic construct
  misc_feature 1..19: note = synthetic oligonucleotide for upregulation of SCN1A expression
  modified_base 1..18: mod_base = OTHER; note = phosphorothioated bases
  source 1..19: mol_type = other DNA
acttcttccactccttcct

SEQ ID NO: 77 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
gatgtcccttcctgcgttgt

SEQ ID NO: 78 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
tgtggatgctgggtgtctctc

SEQ ID NO: 79 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
tcccagtgactcccgatgct

SEQ ID NO: 80 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
agtctcagttgtcagtacctc

SEQ ID NO: 81 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
gttattgaatgccctggtgt

SEQ ID NO: 82 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
tcggatcatcagggttgtagt

SEQ ID NO: 83 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
gtggtataggaactggcagca

SEQ ID NO: 84 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
tctgctcttccctacattgg

SEQ ID NO: 85 - DNA, 19 bp, synthetic construct
  misc_feature 1..19: note = synthetic oligonucleotide for upregulation of SCN1A expression
  modified_base 1..18: mod_base = OTHER; note = phosphorothioated bases
  source 1..19: mol_type = other DNA
gtaatctgctcttccctac

SEQ ID NO: 86 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
gggagaacttgagagcaacag

SEQ ID NO: 87 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
gccagtcacaaattcagatca

SEQ ID NO: 88 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
gtggcatagggacgggcagca

SEQ ID NO: 89 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
gtagcactgtggacatcggc

SEQ ID NO: 90 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
gtagaagaacagcccgtagtg

SEQ ID NO: 91 - DNA, 19 bp, synthetic construct
  misc_feature 1..19: note = synthetic oligonucleotide for upregulation of SCN1A expression
  modified_base 1..18: mod_base = OTHER; note = phosphorothioated bases
  source 1..19: mol_type = other DNA
gtccaatcatacagcagaa

SEQ ID NO: 92 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
gtgactgtaccaattgctgt

SEQ ID NO: 93 - DNA, 19 bp, synthetic construct
  misc_feature 1..19: note = synthetic oligonucleotide for upregulation of SCN1A expression
  modified_base 1..18: mod_base = OTHER; note = phosphorothioated bases
  source 1..19: mol_type = other DNA
acttcttccactccttcct

SEQ ID NO: 94 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
gatgtcccttcctgcgttgt

SEQ ID NO: 95 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
tgtggatgctgggtgtctctc

SEQ ID NO: 96 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
tcccagtgactcccgatgct

SEQ ID NO: 97 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
agtctcagttgtcagtacctc

SEQ ID NO: 98 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
tcggatcatcagggttgtagt

SEQ ID NO: 99 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
gtggtataggaactggcagca

SEQ ID NO: 100 - DNA, 21 bp, synthetic construct
  misc_feature 1..21: note = synthetic oligonucleotide for upregulation of SCN1A expression
  modified_base 1..3: mod_base = OTHER; note = phosphorothioated bases
  modified_base 6: mod_base = OTHER; note = phosphorothioated base
  modified_base 11: mod_base = OTHER; note = phosphorothioated base
  modified_base 15: mod_base = OTHER; note = phosphorothioated base
  modified_base 20: mod_base = OTHER; note = phosphorothioated base
  source 1..21: mol_type = other DNA
gtggacaggatgcacaaagga

SEQ ID NO: 101 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
tggtataggaactggcagca

SEQ ID NO: 102 - DNA, 21 bp, synthetic construct
  misc_feature 1..21: note = synthetic oligonucleotide for upregulation of SCN1A expression
  modified_base 1..20: mod_base = OTHER; note = phosphorothioated bases
  source 1..21: mol_type = other DNA
gtggcatagggacgggcagca

SEQ ID NO: 103 - DNA, 19 bp, synthetic construct
  misc_feature 1..19: note = synthetic oligonucleotide for upregulation of SCN1A expression
  modified_base 1..3: mod_base = OTHER; note = phosphorothioated bases
  modified_base 16..18: mod_base = OTHER; note = phosphorothioated bases
  source 1..19: mol_type = other DNA
gtgactgtgcccattgctg

SEQ ID NO: 104 - DNA, 19 bp, synthetic construct
  misc_feature 1..19: note = synthetic oligonucleotide for upregulation of SCN1A expression
  modified_base 1..3: mod_base = OTHER; note = phosphorothioated bases
  modified_base 7: mod_base = OTHER; note = phosphorothioated bases
  modified_base 13: mod_base = OTHER; note = phosphorothioated bases
  modified_base 16..18: mod_base = OTHER; note = phosphorothioated bases
  source 1..19: mol_type = other DNA
gccacttgatgatctaaac

SEQ ID NO: 105 - DNA, 21 bp, synthetic construct
  misc_feature 1..21: note = synthetic oligonucleotide for upregulation of SCN1A expression
  modified_base 1..3: mod_base = OTHER; note = phosphorothioated bases
  modified_base 6: mod_base = OTHER; note = phosphorothioated bases
  modified_base 11: mod_base = OTHER; note = phosphorothioated bases
  modified_base 15: mod_base = OTHER; note = phosphorothioated bases
  modified_base 20: mod_base = OTHER; note = phosphorothioated bases
  source 1..21: mol_type = other DNA
gtggacaggatgcacaaagga

SEQ ID NO: 106 - DNA, 20 bp, synthetic construct
  misc_feature 1..20: note = synthetic oligonucleotide for upregulation of SCN1A expression
  modified_base 1..19: mod_base = OTHER; note = phosphorothioated bases
  source 1..20: mol_type = other DNA
tggtataggaactggcagca

SEQ ID NO: 107 - DNA, 15 bp, synthetic construct
  misc_feature 1..15: note = synthetic oligonucleotide for upregulation of SCN1A expression
  modified_base 1: mod_base = OTHER; note = locked nucleic acid; phosphorothioated base
  modified_base 2: mod_base = OTHER; note = locked nucleic acid; phosphorothioated base
  modified_base 3..12: mod_base = OTHER; note = phosphorothioated bases
  modified_base 13..14: mod_base = OTHER; note = locked nucleic acid; phosphorothioated base
  modified_base 15: mod_base = OTHER; note = locked nucleic acid
  source 1..15: mol_type = other DNA
ccacgcgcgagtaca

SEQ ID NO: 108 - DNA, 15 bp, synthetic construct
  misc_feature 1..15: note = synthetic oligonucleotide for upregulation of SCN1A expression
  modified_base 1: mod_base = OTHER; note = locked nucleic acid; phosphorothioated base
  modified_base 2: mod_base = OTHER; note = locked nucleic acid; phosphorothioated base
  modified_base 3..12: mod_base = OTHER; note = phosphorothioated bases
  modified_base 13..14: mod_base = OTHER; note = locked nucleic acid; phosphorothioated base
  modified_base 15: mod_base = OTHER; note = locked nucleic acid
  source 1..15: mol_type = other DNA
gtataggaactggca

SEQ ID NO: 109 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 3..6: mod_base = OTHER; note = phosphorothioated bases
  modified_base 7: mod_base = OTHER; note = locked nucleic acid; phosphorothioated base
  modified_base 8..12: mod_base = OTHER; note = phosphorothioated bases
  modified_base 13..14: mod_base = OTHER; note = locked nucleic acid; phosphorothioated base
  modified_base 15: mod_base = OTHER; note = locked nucleic acid
  source 1..15: mol_type = other DNA
gtggtataggaactg

SEQ ID NO: 110 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 3..12: mod_base = OTHER; note = phosphorothioated base
  modified_base 13..14: mod_base = OTHER; note = locked nucleic acid; phosphorothioated base
  modified_base 15: mod_base = OTHER; note = locked nucleic acid
  source 1..15: mol_type = other DNA
agaacttgagagcaa

SEQ ID NO: 111 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 3..5: mod_base = OTHER; note = phosphorothioated base
  modified_base 6: mod_base = OTHER; note = locked nucleic acid; phosphorothioated base
  modified_base 7..8: mod_base = OTHER; note = phosphorothioated base
  modified_base 9: mod_base = OTHER; note = locked nucleic acid; phosphorothioated base
  modified_base 10..12: mod_base = OTHER; note = phosphorothioated base
  modified_base 13..14: mod_base = OTHER; note = locked nucleic acid; phosphorothioated base
  modified_base 15: mod_base = OTHER; note = locked nucleic acid
  source 1..15: mol_type = other DNA
gccagtcacaaattc

SEQ ID NO: 112 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 3..12: mod_base = OTHER; note = phosphorothioated base
  modified_base 13..14: mod_base = OTHER; note = locked nucleic acid; phosphorothioated base
  modified_base 15: mod_base = OTHER; note = locked nucleic acid
  source 1..15: mol_type = other DNA
cacaaattcagatca

SEQ ID NO: 113 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 7: mod_base = OTHER; note = locked nucleic acid; phosphorothioated base
  modified_base 13..14: mod_base = OTHER; note = locked nucleic acid; phosphorothioated base
  modified_base 15: mod_base = OTHER; note = locked nucleic acid
  source 1..15: mol_type = other DNA
gtggtataggaactg

SEQ ID NO: 114 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 3..12: mod_base = OTHER; note = phosphorothioated base
  modified_base 13..14: mod_base = OTHER; note = locked nucleic acid; phosphorothioated base
  modified_base 15: mod_base = OTHER; note = locked nucleic acid
  source 1..15: mol_type = other DNA
gtataggaactggca

SEQ ID NO: 115 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 3..6: mod_base = OTHER; note = phosphorothioated base
  modified_base 7: mod_base = OTHER; note = locked nucleic acid; phosphorothioated base
  modified_base 8..12: mod_base = OTHER; note = phosphorothioated base
  modified_base 13..14: mod_base = OTHER; note = locked nucleic acid; phosphorothioated base
  modified_base 15: mod_base = OTHER; note = locked nucleic acid
  source 1..15: mol_type = other DNA
gtggtataggaactg

SEQ ID NO: 116 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 3..8: mod_base = OTHER; note = phosphorothioated base
  modified_base 9: mod_base = OTHER; note = locked nucleic acid; phosphorothioated base
  modified_base 10..12: mod_base = OTHER; note = phosphorothioated base
  modified_base 13..14: mod_base = OTHER; note = locked nucleic acid; phosphorothioated base
  modified_base 15: mod_base = OTHER; note = locked nucleic acid
  source 1..15: mol_type = other DNA
gccagtcacaaattc

SEQ ID NO: 117 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 13..14: mod_base = OTHER; note = locked nucleic acid; phosphorothioated base
  modified_base 15: mod_base = OTHER; note = locked nucleic acid
  source 1..15: mol_type = other DNA
cacaaattcagatca

SEQ ID NO: 118 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 6: mod_base = OTHER; note = 2'OXethylxodification; phosphorothioated base
  modified_base 9: mod_base = OTHER; note = 2'OXethylxodification; phosphorothioated base
  modified_base 12..13: mod_base = OTHER; note = 2'OXethylxodification; phosphorothioated base
  modified_base 14: mod_base = OTHER; note = locked nucleic acid; phosphorothioated base
  modified_base 15: mod_base = OTHER; note = locked nucleic acid
  source 1..15: mol_type = other DNA
gccagtcacaaattc

SEQ ID NO: 119 - DNA, 15 bp, synthetic construct
  misc_feature 1..15: note = synthetic oligonucleotide for upregulation of SCN1A expression
  modified_base 1: mod_base = OTHER; note = locked nucleic acid; phosphorothioated base
  modified_base 2..5: mod_base = OTHER; note = phosphorothioated base
  modified_base 6: mod_base = OTHER; note = locked nucleic acid; phosphorothioated base
  modified_base 7..8: mod_base = OTHER; note = phosphorothioated base
  modified_base 9: mod_base = OTHER; note = locked nucleic acid; phosphorothioated base
  modified_base 10..13: mod_base = OTHER; note = phosphorothioated base
  modified_base 14: mod_base = OTHER; note = locked nucleic acid; phosphorothioated base
  modified_base 15: mod_base = OTHER; note = locked nucleic acid
  source 1..15: mol_type = other DNA
gccagtcacaaattc

SEQ ID NO: 120 - DNA, 13 bp, synthetic construct
  misc_feature 1..13: note = synthetic oligonucleotide for upregulation of SCN1A expression
  modified_base 1..2: mod_base = OTHER; note = locked nucleic acid; phosphorothioated base
  modified_base 3: mod_base = OTHER; note = 2'OXethylxodification; phosphorothioated base
  modified_base 4..5: mod_base = OTHER; note = phosphorothioated base
  modified_base 6: mod_base = OTHER; note = 2'OXethylxodification; phosphorothioated base
  modified_base 7..8: mod_base = OTHER; note = phosphorothioated base
  modified_base 9: mod_base = OTHER; note = 2'OXethylxodification; phosphorothioated base
  modified_base 10: mod_base = OTHER; note = phosphorothioated base
  modified_base 11: mod_base = OTHER; note = 2'OXethylxodification; phosphorothioated base
  modified_base 12: mod_base = OTHER; note = locked nucleic acid; phosphorothioated base
  modified_base 13: mod_base = OTHER; note = locked nucleic acid
  source 1..13: mol_type = other DNA
gccagtcacaaat

SEQ ID NO: 121 - DNA, 13 bp, synthetic construct
  misc_feature 1..13: note = synthetic oligonucleotide for upregulation of SCN1A expression
  modified_base 1..2: mod_base = OTHER; note = locked nucleic acid; phosphorothioated base
  modified_base 3..10: mod_base = OTHER; note = phosphorothioated base
  modified_base 11..12: mod_base = OTHER; note = locked nucleic acid; phosphorothioated base
  modified_base 13: mod_base = OTHER; note = locked nucleic acid
  source 1..13: mol_type = other DNA
gccagtcacaaat

SEQ ID NO: 122 - DNA, 11 bp, synthetic construct
  misc_feature 1..11: note = synthetic oligonucleotide for upregulation of SCN1A expression
  modified_base 1: mod_base = OTHER; note = locked nucleic acid; phosphorothioated base
  modified_base 2..8: mod_base = OTHER; note = phosphorothioated base
  modified_base 9..10: mod_base = OTHER; note = locked nucleic acid; phosphorothioated base
  modified_base 11: mod_base = OTHER; note = locked nucleic acid
  source 1..11: mol_type = other DNA
gccagtcacaa

SEQ ID NO: 123 - DNA, 12 bp, synthetic construct
  misc_feature 1..12: note = synthetic oligonucleotide for upregulation of SCN1A expression
  modified_base 1..2: mod_base = OTHER; note = locked nucleic acid; phosphorothioated base
  modified_base 3..9: mod_base = OTHER; note = phosphorothioated base
  modified_base 10..11: mod_base = OTHER; note = locked nucleic acid; phosphorothioated base
  modified_base 12: mod_base = OTHER; note = locked nucleic acid
  source 1..12: mol_type = other DNA
gccagtcacaaa

SEQ ID NO: 124 - DNA, 12 bp, synthetic construct
  misc_feature 1..12: note = synthetic oligonucleotide for upregulation of SCN1A expression
  modified_base 1..2: mod_base = OTHER; note = locked nucleic acid; phosphorothioated base
  modified_base 3..9: mod_base = OTHER; note = phosphorothioated base
  modified_base 10..11: mod_base = OTHER; note = locked nucleic acid; phosphorothioated base
  modified_base 12: mod_base = OTHER; note = locked nucleic acid
  source 1..12: mol_type = other DNA
attgagccagtc

SEQ ID NO: 125 - DNA, 15 bp, synthetic construct
  misc_feature 1..15: note = synthetic oligonucleotide for upregulation of SCN1A expression
  modified_base 1..2: mod_base = OTHER; note = locked nucleic acid; phosphorothioated base
  modified_base 3..6: mod_base = OTHER; note = phosphorothioated base
  modified_base 7: mod_base = OTHER; note = locked nucleic acid; phosphorothioated base
  modified_base 8..12: mod_base = OTHER; note = phosphorothioated base
  modified_base 13..14: mod_base = OTHER; note = locked nucleic acid; phosphorothioated base
  modified_base 15: mod_base = OTHER; note = locked nucleic acid
  source 1..15: mol_type = other DNA
gtggtataggaactg

SEQ ID NO: 126 - DNA, 17 bp, synthetic construct
  misc_feature 1..17: note = synthetic oligonucleotide for upregulation of SCN1A expression
  modified_base 1..2: mod_base = OTHER; note = 2'OXethylxodification; phosphorothioated base
  modified_base 3..5: mod_base = OTHER; note = phosphorothioated base
  modified_base 6: mod_base = OTHER; note = 2'OXethylxodification; phosphorothioated base
  modified_base 7..8: mod_base = OTHER; note = phosphorothioated base
  modified_base 9: mod_base = OTHER; note = 2'OXethylxodification; phosphorothioated base
  modified_base 10..12: mod_base = OTHER; note = phosphorothioated base
  modified_base 13: mod_base = OTHER; note = 2'OXethylxodification; phosphorothioated base
  modified_base 14..15: mod_base = OTHER; note = phosphorothioated base
  modified_base 16: mod_base = OTHER; note = 2'OXethylxodification; phosphorothioated base
  modified_base 17: mod_base = OTHER; note = 2'OXethylxodification
  source 1..17: mol_type = other DNA
gccagtcacaaattcag

SEQ ID NO: 127 - DNA, 15 bp, synthetic construct
  misc_feature 1..15: note = synthetic oligonucleotide for upregulation of SCN1A expression
  modified_base 1..2: mod_base = OTHER; note = 2'OXethylxodification; phosphorothioated base
  modified_base 3..5: mod_base = OTHER; note = phosphorothioated base
  modified_base 6: mod_base = OTHER; note = 2'OXethylxodification; phosphorothioated base
  modified_base 7..8: mod_base = OTHER; note = phosphorothioated base
  modified_base 9: mod_base = OTHER; note = 2'OXethylxodification; phosphorothioated base
  modified_base 10..12: mod_base = OTHER; note = phosphorothioated base
  modified_base 13..14: mod_base = OTHER; note = 2'OXethylxodification; phosphorothioated base
  modified_base 15: mod_base = OTHER; note = 2'OXethylxodification
  source 1..15: mol_type = other DNA
gccagtcacaaattc

SEQ ID NO: 128 - DNA, 21 bp, synthetic construct
  misc_feature 1..21: note = synthetic oligonucleotide for upregulation of SCN1A expression
  modified_base 1..3: mod_base = OTHER; note = 2'OXethylxodification; phosphorothioated base
  modified_base 4: mod_base = OTHER; note = phosphorothioated base
  modified_base 5: mod_base = OTHER; note = 2'OXethylxodification; phosphorothioated base
  modified_base 6: mod_base = OTHER; note = phosphorothioated base
  modified_base 7: mod_base = OTHER; note = 2'OXethylxodification; phosphorothioated base
  modified_base 8..12: mod_base = OTHER; note = phosphorothioated base
  modified_base 13: mod_base = OTHER; note = 2'OXethylxodification; phosphorothioated base
  modified_base 14..16: mod_base = OTHER; note = phosphorothioated base
  modified_base 17: mod_base = OTHER; note = 2'OXethylxodification; phosphorothioated base
  modified_base 18: mod_base = OTHER; note = phosphorothioated base
  modified_base 19..20: mod_base = OTHER; note = 2'OXethylxodification; phosphorothioated base
  modified_base 21: mod_base = OTHER; note = 2'OXethylxodification
  source 1..21: mol_type = other DNA
gtggtataggaactggcagca

SEQ ID NO: 129 - DNA, 21 bp, synthetic construct
  misc_feature 1..21: note = synthetic oligonucleotide for upregulation of SCN1A expression
  modified_base 1..3: mod_base = OTHER; note = 2'OXethylxodification; phosphorothioated base
  modified_base 4..7: mod_base = OTHER; note = phosphorothioated base
  modified_base 8: mod_base = OTHER; note = 2'OXethylxodification; phosphorothioated base
  modified_base 9: mod_base = OTHER; note = phosphorothioated base
  modified_base 10: mod_base = OTHER; note = 2'OXethylxodification; phosphorothioated base
  modified_base 11..15: mod_base = OTHER; note = phosphorothioated base
  modified_base 16: mod_base = OTHER; note = 2'OXethylxodification; phosphorothioated base
  modified_base 17..18: mod_base = OTHER; note = phosphorothioated base
  modified_base 19..20: mod_base = OTHER; note = 2'OXethylxodification; phosphorothioated base
  modified_base 21: mod_base = OTHER; note = 2'OXethylxodification
  source 1..21: mol_type = other DNA
gggagaacttgagagcaacag

SEQ ID NO: 130 - DNA, 21 bp, synthetic construct
  misc_feature 1..21: note = synthetic oligonucleotide for upregulation of SCN1A expression
  modified_base 1..3: mod_base = OTHER; note = 2'OXethylxodification; phosphorothioated base
  modified_base 4..6: mod_base = OTHER; note = phosphorothioated base
  modified_base 7: mod_base = OTHER; note = 2'OXethylxodification; phosphorothioated base
  modified_base 8..12: mod_base = OTHER; note = phosphorothioated base
  modified_base 13: mod_base = OTHER; note = 2'OXethylxodification; phosphorothioated base
  modified_base 14: mod_base = OTHER; note = phosphorothioated base
  modified_base 15: mod_base = OTHER; note = 2'OXethylxodification; phosphorothioated base
  modified_base 16..18: mod_base = OTHER; note = phosphorothioated base
  modified_base 19..20: mod_base = OTHER; note = 2'OXethylxodification; phosphorothioated base
  modified_base 21: mod_base = OTHER; note = 2'OXethylxodification
  source 1..21: mol_type = other DNA
gccagtcacaaattcagatca

SEQ ID NO: 131 - DNA, 21 bp, synthetic construct
  misc_feature 1..21: note = synthetic oligonucleotide for upregulation of SCN1A expression
  modified_base 1..3: mod_base = OTHER; note = 2'OXethylxodification; phosphorothioated base
  modified_base 5: mod_base = OTHER; note = 2'OXethylxodification; phosphorothioated base
  modified_base 7: mod_base = OTHER; note = 2'OXethylxodification; phosphorothioated base
  modified_base 13: mod_base = OTHER; note = 2'OXethylxodification; phosphorothioated base
  modified_base 17: mod_base = OTHER; note = 2'OXethylxodification; phosphorothioated base
  modified_base 19..20: mod_base = OTHER; note = 2'OXethylxodification; phosphorothioated base
  modified_base 21: mod_base = OTHER; note = 2'OXethylxodification
  source 1..21: mol_type = other DNA
gtggtataggaactggcagca

SEQ ID NO: 132 - DNA, 25 bp, synthetic construct
  misc_feature 1..25: note = synthetic oligonucleotide for upregulation of SCN1A expression
  modified_base 1..3: mod_base = OTHER; note = 2'OXethylxodification; phosphorothioated base
  modified_base 4: mod_base = OTHER; note = phosphorothioated base
  modified_base 5: mod_base = OTHER; note = 2'OXethylxodification; phosphorothioated base
  modified_base 6..8: mod_base = OTHER; note = phosphorothioated base
  modified_base 9: mod_base = OTHER; note = 2'OXethylxodification; phosphorothioated base
  modified_base 10..11: mod_base = OTHER; note = phosphorothioated base
  modified_base 12: mod_base = OTHER; note = 2'OXethylxodification; phosphorothioated base
  modified_base 13..14: mod_base = OTHER; note = phosphorothioated base
  modified_base 15: mod_base = OTHER; note = 2'OXethylxodification; phosphorothioated base
  modified_base 16..17: mod_base = OTHER; note = phosphorothioated base
  modified_base 18: mod_base = OTHER; note = 2'OXethylxodification; phosphorothioated base
  modified_base 19..20: mod_base = OTHER; note = phosphorothioated base
  modified_base 21: mod_base = OTHER; note = 2'OXethylxodification; phosphorothioated base
  modified_base 22: mod_base = OTHER; note = phosphorothioated base
  modified_base 23..24: mod_base = OTHER; note = 2'OXethylxodification; phosphorothioated base
  modified_base 25: mod_base = OTHER; note = 2'OXethylxodification
  source 1..25: mol_type = other DNA
ggtataggaactggcagcagtgttg

SEQ ID NO: 133 - DNA, 22 bp, synthetic construct
  misc_feature 1..22: note = synthetic oligonucleotide for upregulation of SCN1A expression
  modified_base 1..3: mod_base = OTHER; note = 2'OXethylxodification; phosphorothioated base
  modified_base 4..5: mod_base = OTHER; note = phosphorothioated base
  modified_base 6: mod_base = OTHER; note = 2'OXethylxodification; phosphorothioated base
  modified_base 7..8: mod_base = OTHER; note = phosphorothioated base
  modified_base 9: mod_base = OTHER; note = 2'OXethylxodification; phosphorothioated base
  modified_base 10..11: mod_base = OTHER; note = phosphorothioated base
  modified_base 12: mod_base = OTHER; note = 2'OXethylxodification; phosphorothioated base
  modified_base 13..15: mod_base = OTHER; note = phosphorothioated base
  modified_base 16: mod_base = OTHER; note = 2'OXethylxodification; phosphorothioated base
  modified_base 17..19: mod_base = OTHER; note = phosphorothioated base
  modified_base 20..21: mod_base = OTHER; note = 2'OXethylxodification; phosphorothioated base
  modified_base 22: mod_base = OTHER; note = 2'OXethylxodification
  source 1..22: mol_type = other DNA
tggtataggaactggcagcagt

SEQ ID NO: 134 - DNA, 25 bp, synthetic construct
  misc_feature 1..25: note = synthetic oligonucleotide for upregulation of SCN1A expression
  modified_base 1: mod_base = OTHER; note = 2'OXethylxodification; phosphorothioated base
  modified_base 2..4: mod_base = OTHER; note = phosphorothioated base
  modified_base 5: mod_base = OTHER; note = 2'OXethylxodification; phosphorothioated base
  modified_base 6..10: mod_base = OTHER; note = phosphorothioated base
  modified_base 11: mod_base = OTHER; note = 2'OXethylxodification; phosphorothioated base
  modified_base 12..14: mod_base = OTHER; note = phosphorothioated base
  modified_base 15: mod_base = OTHER; note = 2'OXethylxodification; phosphorothioated base
  modified_base 16..17: mod_base = OTHER; note = phosphorothioated base
  modified_base 18: mod_base = OTHER; note = 2'OXethylxodification; phosphorothioated base
  modified_base 19..24: mod_base = OTHER; note = phosphorothioated base
  modified_base 25: mod_base = OTHER; note = 2'OXethylxodification
  source 1..25: mol_type = other DNA
ggtataggaactggcagcagtgttg

SEQ ID NO: 135 - DNA, 24 bp, synthetic construct
  misc_feature 1..24: note = synthetic oligonucleotide for upregulation of SCN1A expression
  modified_base 1..2: mod_base = OTHER; note = 2'OXethylxodification; phosphorothioated base
  modified_base 3: mod_base = OTHER; note = phosphorothioated base
  modified_base 4: mod_base = OTHER; note = 2'OXethylxodification; phosphorothioated base
  modified_base 5..6: mod_base = OTHER; note = phosphorothioated base
  modified_base 7: mod_base = OTHER; note = 2'OXethylxodification; phosphorothioated base
  modified_base 8..14: mod_base = OTHER; note = phosphorothioated base
  modified_base 15: mod_base = OTHER; note = 2'OXethylxodification; phosphorothioated base
  modified_base 16..18: mod_base = OTHER; note = phosphorothioated base
  modified_base 19: mod_base = OTHER; note = 2'OXethylxodification; phosphorothioated base
  modified_base 20..21: mod_base = OTHER; note = phosphorothioated base
  modified_base 22: mod_base = OTHER; note = 2'OXethylxodification; phosphorothioated base
  modified_base 23: mod_base = OTHER; note = phosphorothioated base
  modified_base 24: mod_base = OTHER; note = 2'OXethylxodification
  source 1..24: mol_type = other DNA
aagcggtataggaactggcagcag

SEQ ID NO: 136 - DNA, 26 bp, synthetic construct
  misc_feature 1..26: note = synthetic oligonucleotide for upregulation of SCN1A expression
  modified_base 1..3: mod_base = OTHER; note = 2'OXethylxodification; phosphorothioated base
  modified_base 4..7: mod_base = OTHER; note = phosphorothioated base
  modified_base 8: mod_base = OTHER; note = 2'OXethylxodification; phosphorothioated base
  modified_base 9..10: mod_base = OTHER; note = phosphorothioated base
  modified_base 11: mod_base = OTHER; note = 2'OXethylxodification; phosphorothioated base
  modified_base 12..14: mod_base = OTHER; note = phosphorothioated base
  modified_base 15: mod_base = OTHER; note = 2'OXethylxodification; phosphorothioated base
  modified_base 16..19: mod_base = OTHER; note = phosphorothioated base
  modified_base 20: mod_base = OTHER; note = 2'OXethylxodification; phosphorothioated base
  modified_base 21..23: mod_base = OTHER; note = phosphorothioated base
  modified_base 24..25: mod_base = OTHER; note = 2'OXethylxodification; phosphorothioated base
  modified_base 26: mod_base = OTHER; note = 2'OXethylxodification
  source 1..26: mol_type = other DNA
gagccagtcacaaattcagatcaccc

SEQ ID NO: 137 - DNA, 21 bp, synthetic construct
  misc_feature 1..21: note = synthetic oligonucleotide for upregulation of SCN1A expression
  modified_base 1: mod_base = OTHER; note = 2'OXethylxodification; phosphorothioated base
  modified_base 2: mod_base = OTHER; note = phosphorothioated base
  modified_base 3: mod_base = OTHER; note = 2'OXethylxodification; phosphorothioated base
  modified_base 4..10: mod_base = OTHER; note = phosphorothioated base
  modified_base 11..13: mod_base = OTHER; note = 2'OXethylxodification; phosphorothioated base
  modified_base 14..18: mod_base = OTHER; note = phosphorothioated base
  modified_base 19..20: mod_base = OTHER; note = 2'OXethylxodification; phosphorothioated base
  modified_base 21: mod_base = OTHER; note = 2'OXethylxodification
  source 1..21: mol_type = other DNA
aatgggagaacttgagagcaa

SEQ ID NO: 138 - DNA, 19 bp, synthetic construct
  misc_feature 1..19: note = synthetic oligonucleotide for upregulation of SCN1A expression
  modified_base 1: mod_base = OTHER; note = 2'OXethylxodification; phosphorothioated base
  modified_base 6: mod_base = OTHER; note = 2'OXethylxodification; phosphorothioated base
  modified_base 12: mod_base = OTHER; note = 2'OXethylxodification; phosphorothioated base
  modified_base 19: mod_base = OTHER; note = 2'OXethylxodification
  source 1..19: mol_type = other DNA
gtgactgtgcccattgctg

SEQ ID NO: 139 - DNA, 26 bp, synthetic construct
  misc_feature 1..26: note = synthetic oligonucleotide for upregulation of SCN1A expression
  modified_base 1: mod_base = OTHER; note = 2'OXethylxodification; phosphorothioated base
  modified_base 6: mod_base = OTHER; note = 2'OXethylxodification; phosphorothioated base
  modified_base 11: mod_base = OTHER; note = 2'OXethylxodification; phosphorothioated base
  modified_base 17: mod_base = OTHER; note = 2'OXethylxodification; phosphorothioated base
  modified_base 21: mod_base = OTHER; note = 2'OXethylxodification; phosphorothioated base
  modified_base 26: mod_base = OTHER; note = 2'OXethylxodification
  source 1..26: mol_type = other DNA
gacaaccttgcagccactgatgatga

SEQ ID NO: 140 - DNA, 20 bp, synthetic construct
  misc_feature 1..20: note = synthetic oligonucleotide for upregulation of SCN1A expression
  modified_base 1..2: mod_base = OTHER; note = 2'OXethylxodification; phosphorothioated base
  modified_base 3: mod_base = OTHER; note = phosphorothioated base
  modified_base 4: mod_base = OTHER; note = 2'OXethylxodification; phosphorothioated base
  modified_base 5: mod_base = OTHER; note = phosphorothioated base
  modified_base 6: mod_base = OTHER; note = 2'OXethylxodification; phosphorothioated base
  modified_base 7..11: mod_base = OTHER; note = phosphorothioated base
  modified_base 12: mod_base = OTHER; note = 2'OXethylxodification; phosphorothioated base
  modified_base 13..15: mod_base = OTHER; note = phosphorothioated base
  modified_base 16: mod_base = OTHER; note = 2'OXethylxodification; phosphorothioated base
  modified_base 17: mod_base = OTHER; note = phosphorothioated base
  modified_base 18..19: mod_base = OTHER; note = 2'OXethylxodification; phosphorothioated base
  modified_base 20: mod_base = OTHER; note = 2'OXethylxodification
  source 1..20: mol_type = other DNA
tggtataggaactggcagca

SEQ ID NO: 141 - DNA, 20 bp, synthetic construct
  misc_feature 1..20: note = synthetic oligonucleotide for upregulation of SCN1A expression
  modified_base 1..2: mod_base = OTHER; note = 2'OXethylxodification; phosphorothioated base
  modified_base 3..5: mod_base = OTHER; note = phosphorothioated base
  modified_base 6: mod_base = OTHER; note = 2'OXethylxodification; phosphorothioated base
  modified_base 7..11: mod_base = OTHER; note = phosphorothioated base
  modified_base 12: mod_base = OTHER; note = 2'OXethylxodification; phosphorothioated base
  modified_base 13: mod_base = OTHER; note = phosphorothioated base
  modified_base 14: mod_base = OTHER; note = 2'OXethylxodification; phosphorothioated base
  modified_base 15..17: mod_base = OTHER; note = phosphorothioated base
  modified_base 18..19: mod_base = OTHER; note = 2'OXethylxodification; phosphorothioated base
  modified_base 20: mod_base = OTHER; note = 2'OXethylxodification
  source 1..20: mol_type = other DNA
ccagtcacaaattcagatca

SEQ ID NO: 142 - DNA, 20 bp, synthetic construct
  misc_feature 1..20: note = synthetic oligonucleotide for upregulation of SCN1A expression
  modified_base 1..2: mod_base = OTHER; note = 2'OXethylxodification; phosphorothioated base
  modified_base 4: mod_base = OTHER; note = 2'OXethylxodification; phosphorothioated base
  modified_base 6: mod_base = OTHER; note = 2'OXethylxodification; phosphorothioated base
  modified_base 12: mod_base = OTHER; note = 2'OXethylxodification; phosphorothioated base
  modified_base 16: mod_base = OTHER; note = 2'OXethylxodification; phosphorothioated base
  modified_base 18..19: mod_base = OTHER; note = 2'OXethylxodification; phosphorothioated base
  modified_base 20: mod_base = OTHER; note = 2'OXethylxodification
  source 1..20: mol_type = other DNA
tggtataggaactggcagca

SEQ ID NO: 143 - DNA, 25 bp, synthetic construct
  misc_feature 1..25: note = synthetic oligonucleotide for upregulation of SCN1A expression
  modified_base 1..2: mod_base = OTHER; note = 2'OXethylxodification; phosphorothioated base
  modified_base 3..6: mod_base = OTHER; note = phosphorothioated base
  modified_base 7: mod_base = OTHER; note = 2'OXethylxodification; phosphorothioated base
  modified_base 8..9: mod_base = OTHER; note = phosphorothioated base
  modified_base 10: mod_base = OTHER; note = 2'OXethylxodification; phosphorothioated base
  modified_base 11..13: mod_base = OTHER; note = phosphorothioated base
  modified_base 14: mod_base = OTHER; note = 2'OXethylxodification; phosphorothioated base
  modified_base 15..18: mod_base = OTHER; note = phosphorothioated base
  modified_base 19: mod_base = OTHER; note = 2'OXethylxodification; phosphorothioated base
  modified_base 20..22: mod_base = OTHER; note = phosphorothioated base
  modified_base 23..24: mod_base = OTHER; note = 2'OXethylxodification; phosphorothioated base
  modified_base 25: mod_base = OTHER; note = 2'OXethylxodification
  source 1..25: mol_type = other DNA
agccagtcacaaattcagatcaccc

SEQ ID NO: 144 - DNA, 17 bp, synthetic construct
  misc_feature 1..17: note = synthetic oligonucleotide for upregulation of SCN1A expression
  modified_base 1: mod_base = OTHER; note = 2'OXethylxodification; phosphorothioated base
  modified_base 2: mod_base = OTHER; note = phosphorothioated base
  modified_base 3: mod_base = OTHER; note = 2'OXethylxodification; phosphorothioated base
  modified_base 4..8: mod_base = OTHER; note = phosphorothioated base
  modified_base 9: mod_base = OTHER; note = 2'OXethylxodification; phosphorothioated base
  modified_base 10..12: mod_base = OTHER; note = phosphorothioated base
  modified_base 13: mod_base = OTHER; note = 2'OXethylxodification; phosphorothioated base
  modified_base 14: mod_base = OTHER; note = phosphorothioated base
  modified_base 15..16: mod_base = OTHER; note = 2'OXethylxodification; phosphorothioated base
  modified_base 17: mod_base = OTHER; note = 2'OXethylxodification
  source 1..17: mol_type = other DNA
tataggaactggcagca

SEQ ID NO: 145 - DNA, 21 bp, synthetic construct
  misc_feature 1..21: note = synthetic oligonucleotide for upregulation of SCN1A expression
  modified_base 1..3: mod_base = OTHER; note = 2'OXethylxodification; phosphorothioated base
  modified_base 5: mod_base = OTHER; note = 2'OXethylxodification; phosphorothioated base
  modified_base 7: mod_base = OTHER; note = 2'OXethylxodification; phosphorothioated base
  modified_base 13: mod_base = OTHER; note = 2'OXethylxodification; phosphorothioated base
  modified_base 17: mod_base = OTHER; note = 2'OXethylxodification; phosphorothioated base
  modified_base 19..20: mod_base = OTHER; note = 2'OXethylxodification; phosphorothioated base
  modified_base 21: mod_base = OTHER; note = 2'OXethylxodification
  source 1..21: mol_type = other DNA
gtggtataggaactggcagca

SEQ ID NO: 146 - DNA, 25 bp, synthetic construct
  misc_feature 1..25: note = synthetic oligonucleotide for upregulation of SCN1A expression
  modified_base 1..3: mod_base = OTHER; note = 2'OXethylxodification; phosphorothioated base
  modified_base 4: mod_base = OTHER; note = phosphorothioated base
  modified_base 5: mod_base = OTHER; note = 2'OXethylxodification; phosphorothioated base
  modified_base 6..8: mod_base = OTHER; note = phosphorothioated base
  modified_base 9: mod_base = OTHER; note = 2'OXethylxodification; phosphorothioated base
  modified_base 10..11: mod_base = OTHER; note = phosphorothioated base
  modified_base 12: mod_base = OTHER; note = 2'OXethylxodification; phosphorothioated base
  modified_base 13..14: mod_base = OTHER; note = phosphorothioated base
  modified_base 15: mod_base = OTHER; note = 2'OXethylxodification; phosphorothioated base
  modified_base 16..17: mod_base = OTHER; note = phosphorothioated base
  modified_base 18: mod_base = OTHER; note = 2'OXethylxodification; phosphorothioated base
  modified_base 19..20: mod_base = OTHER; note = phosphorothioated base
  modified_base 21: mod_base = OTHER; note = 2'OXethylxodification; phosphorothioated base
  modified_base 22: mod_base = OTHER; note = phosphorothioated base
  modified_base 23..24: mod_base = OTHER; note = 2'OXethylxodification; phosphorothioated base
  modified_base 25: mod_base = OTHER; note = 2'OXethylxodification
  source 1..25: mol_type = other DNA
ggtataggaactggcagcagtgttg

SEQ ID NO: 147 - DNA, 22 bp, synthetic construct
  misc_feature 1..22: note = synthetic oligonucleotide for upregulation of SCN1A expression
  modified_base 1..3: mod_base = OTHER; note = 2'OXethylxodification; phosphorothioated base
  modified_base 4..5: mod_base = OTHER; note = phosphorothioated base
  modified_base 6: mod_base = OTHER; note = 2'OXethylxodification; phosphorothioated base
  modified_base 7..8: mod_base = OTHER; note = phosphorothioated base
  modified_base 9: mod_base = OTHER; note = 2'OXethylxodification; phosphorothioated base
  modified_base 10..11: mod_base = OTHER; note = phosphorothioated base
  modified_base 12: mod_base = OTHER; note = 2'OXethylxodification; phosphorothioated base
  modified_base 13..15: mod_base = OTHER; note = phosphorothioated base
  modified_base 16: mod_base = OTHER; note = 2'OXethylxodification; phosphorothioated base
  modified_base 17..19: mod_base = OTHER; note = phosphorothioated base
  modified_base 20..21: mod_base = OTHER; note = 2'OXethylxodification; phosphorothioated base
  modified_base 22: mod_base = OTHER; note = 2'OXethylxodification
  source 1..22: mol_type = other DNA
tggtataggaactggcagcagt

SEQ ID NO: 148 - DNA, 25 bp, synthetic construct
  misc_feature 1..25: note = synthetic oligonucleotide for upregulation of SCN1A expression
  modified_base 1: mod_base = OTHER; note = 2'OXethylxodification; phosphorothioated base
  modified_base 2..4: mod_base = OTHER; note = phosphorothioated base
  modified_base 5: mod_base = OTHER; note = 2'OXethylxodification; phosphorothioated base
  modified_base 6..10: mod_base = OTHER; note = phosphorothioated base
  modified_base 11: mod_base = OTHER; note = 2'OXethylxodification; phosphorothioated base
  modified_base 12..14: mod_base = OTHER; note = phosphorothioated base
  modified_base 15: mod_base = OTHER; note = 2'OXethylxodification; phosphorothioated base
  modified_base 16..17: mod_base = OTHER; note = phosphorothioated base
  modified_base 18: mod_base = OTHER; note = 2'OXethylxodification; phosphorothioated base
  modified_base 19..24: mod_base = OTHER; note = phosphorothioated base
  modified_base 25: mod_base = OTHER; note = 2'OXethylxodification
  source 1..25: mol_type = other DNA
ggtataggaactggcagcagtgttg

SEQ ID NO: 149 - DNA, 24 bp, synthetic construct
  misc_feature 1..24: note = synthetic oligonucleotide for upregulation of SCN1A expression
  modified_base 1..2: mod_base = OTHER; note = 2'OXethylxodification; phosphorothioated base
  modified_base 3: mod_base = OTHER; note = phosphorothioated base
  modified_base 4: mod_base = OTHER; note = 2'OXethylxodification; phosphorothioated base
  modified_base 5..6: mod_base = OTHER; note = phosphorothioated base
  modified_base 7: mod_base = OTHER; note = 2'OXethylxodification; phosphorothioated base
  modified_base 8..14: mod_base = OTHER; note = phosphorothioated base
  modified_base 15: mod_base = OTHER; note = 2'OXethylxodification; phosphorothioated base
  modified_base 16..18: mod_base = OTHER; note = phosphorothioated base
  modified_base 19: mod_base = OTHER; note = 2'OXethylxodification; phosphorothioated base
  modified_base 20..21: mod_base = OTHER; note = phosphorothioated base
  modified_base 22: mod_base = OTHER; note = 2'OXethylxodification; phosphorothioated base
  modified_base 23: mod_base = OTHER; note = phosphorothioated base
  modified_base 24: mod_base = OTHER; note = 2'OXethylxodification
  source 1..24: mol_type = other DNA
aagcggtataggaactggcagcag

SEQ ID NO: 150 - DNA, 21 bp, synthetic construct
  misc_feature 1..21: note = synthetic oligonucleotide for upregulation of SCN1A expression
  modified_base 1..3: mod_base = OTHER; note = 2'OXethylxodification; phosphorothioated base
  modified_base 4: mod_base = OTHER; note = phosphorothioated base
  modified_base 5: mod_base = OTHER; note = 2'OXethylxodification; phosphorothioated base
  modified_base 6: mod_base = OTHER; note = phosphorothioated base
  modified_base 7: mod_base = OTHER; note = 2'OXethylxodification; phosphorothioated base
  modified_base 8..9: mod_base = OTHER; note = phosphorothioated base
  modified_base 10: mod_base = OTHER; note = 2'OXethylxodification; phosphorothioated base
  modified_base 11..12: mod_base = OTHER; note = phosphorothioated base
  modified_base 13: mod_base = OTHER; note = 2'OXethylxodification; phosphorothioated base
  modified_base 14..16: mod_base = OTHER; note = phosphorothioated base
  modified_base 17: mod_base = OTHER; note = 2'OXethylxodification; phosphorothioated base
  modified_base 18: mod_base = OTHER; note = phosphorothioated base
  modified_base 19..20: mod_base = OTHER; note = 2'OXethylxodification; phosphorothioated base
  modified_base 21: mod_base = OTHER; note = 2'OXethylxodification
  source 1..21: mol_type = other DNA
gtggcatagggacgggcagca

SEQ ID NO: 151 - DNA, 25 bp, synthetic construct
  misc_feature 1..25: note = synthetic oligonucleotide for upregulation of SCN1A expression
  modified_base 1..6: mod_base = OTHER; note = 2'OXethylxodification; phosphorothioated base
  modified_base 7..19: mod_base = OTHER; note = phosphorothioated base
  modified_base 20..24: mod_base = OTHER; note = 2'OXethylxodification; phosphorothioated base
  modified_base 25: mod_base = OTHER; note = 2'OXethylxodification
  source 1..25: mol_type = other DNA
acaagtggcatagggacgggcagca

SEQ ID NO: 152 - DNA, 25 bp, synthetic construct
  misc_feature 1..25: note = synthetic oligonucleotide for upregulation of SCN1A expression
  modified_base 1..2: mod_base = OTHER; note = 2'OXethylxodification; phosphorothioated base
  modified_base 3..5: mod_base = OTHER; note = phosphorothioated base
  modified_base 6: mod_base = OTHER; note = 2'OXethylxodification; phosphorothioated base
  modified_base 7..8: mod_base = OTHER; note = phosphorothioated base
  modified_base 9: mod_base = OTHER; note = 2'OXethylxodification; phosphorothioated base
  modified_base 10..12: mod_base = OTHER; note = phosphorothioated base
  modified_base 13: mod_base = OTHER; note = 2'OXethylxodification; phosphorothioated base
  modified_base 14..16: mod_base = OTHER; note = phosphorothioated base
  modified_base 17: mod_base = OTHER; note = 2'OXethylxodification; phosphorothioated base
  modified_base 18..20: mod_base = OTHER; note = phosphorothioated base
  modified_base 21: mod_base = OTHER; note = 2'OXethylxodification; phosphorothioated base
  modified_base 22..23: mod_base = OTHER; note = phosphorothioated base
  modified_base 24: mod_base = OTHER; note = 2'OXethylxodification; phosphorothioated base
  modified_base 25: mod_base = OTHER; note = 2'OXethylxodification
  source 1..25: mol_type = other DNA
acaagtggcatagggacgggcagca

SEQ ID NO: 153 - DNA, 25 bp, synthetic construct
  misc_feature 1..25: note = synthetic oligonucleotide for upregulation of SCN1A expression
  modified_base 1: mod_base = OTHER; note = 2'OXethylxodification; phosphorothioated base
  modified_base 2..3: mod_base = OTHER; note = phosphorothioated base
  modified_base 4: mod_base = OTHER; note = 2'OXethylxodification; phosphorothioated base
  modified_base 5..6: mod_base = OTHER; note = phosphorothioated base
  modified_base 7: mod_base = OTHER; note = 2'OXethylxodification; phosphorothioated base
  modified_base 8: mod_base = OTHER; note = phosphorothioated base
  modified_base 9: mod_base = OTHER; note = 2'OXethylxodification; phosphorothioated base
  modified_base 10..11: mod_base = OTHER; note = phosphorothioated base
  modified_base 12: mod_base = OTHER; note = 2'OXethylxodification; phosphorothioated base
  modified_base 13..14: mod_base = OTHER; note = phosphorothioated base
  modified_base 15: mod_base = OTHER; note = 2'OXethylxodification; phosphorothioated base
  modified_base 16..18: mod_base = OTHER; note = phosphorothioated base
  modified_base 19: mod_base = OTHER; note = 2'OXethylxodification; phosphorothioated base
  modified_base 20..21: mod_base = OTHER; note = phosphorothioated base
  modified_base 22: mod_base = OTHER; note = 2'OXethylxodification; phosphorothioated base
  modified_base 23..24: mod_base = OTHER; note = phosphorothioated base
  modified_base 25: mod_base = OTHER; note = 2'OXethylxodification
  source 1..25: mol_type = other DNA
aagtggcatagggacgggcagcagt

SEQ ID NO: 154 - DNA, 20 bp, synthetic construct
  misc_feature 1..20: note = synthetic oligonucleotide for upregulation of SCN1A expression
  modified_base 1..2: mod_base = OTHER; note = 2'OXethylxodification; phosphorothioated base
  modified_base 3: mod_base = OTHER; note = phosphorothioated base
  modified_base 4: mod_base = OTHER; note = 2'OXethylxodification; phosphorothioated base
  modified_base 5: mod_base = OTHER; note = phosphorothioated base
  modified_base 6: mod_base = OTHER; note = 2'OXethylxodification; phosphorothioated base
  modified_base 7..11: mod_base = OTHER; note = phosphorothioated base
  modified_base 12: mod_base = OTHER; note = 2'OXethylxodification; phosphorothioated base
  modified_base 13..15: mod_base = OTHER; note = phosphorothioated base
  modified_base 16: mod_base = OTHER; note = 2'OXethylxodification; phosphorothioated base
  modified_base 17: mod_base = OTHER; note = phosphorothioated base
  modified_base 18..19: mod_base = OTHER; note = 2'OXethylxodification; phosphorothioated base
  modified_base 20: mod_base = OTHER; note = 2'OXethylxodification
  source 1..20: mol_type = other DNA
tggtataggaactggcagca

SEQ ID NO: 155 - DNA, 24 bp, synthetic construct
  misc_feature 1..24: note = synthetic oligonucleotide for upregulation of SCN1A expression
  modified_base 1..6: mod_base = OTHER; note = 2'OXethylxodification; phosphorothioated base
  modified_base 7..18: mod_base = OTHER; note = phosphorothioated base
  modified_base 19..23: mod_base = OTHER; note = 2'OXethylxodification; phosphorothioated base
  modified_base 24: mod_base = OTHER; note = 2'OXethylxodification
  source 1..24: mol_type = other DNA
cctatctttcccccccctaccttt

SEQ ID NO: 156 - DNA, 25 bp, synthetic construct
  misc_feature 1..25: note = synthetic oligonucleotide for upregulation of SCN1A expression
  modified_base 1..5: mod_base = OTHER; note = 2'OXethylxodification; phosphorothioated base
  modified_base 6..20: mod_base = OTHER; note = phosphorothioated base
  modified_base 21..24: mod_base = OTHER; note = 2'OXethylxodification; phosphorothioated base
  modified_base 25: mod_base = OTHER; note = 2'OXethylxodification
  source 1..25: mol_type = other DNA
aagtggcatagggacgggcagcagt

SEQ ID NO: 157 - DNA, 19 bp, synthetic construct
  misc_feature 1..19: note = synthetic oligonucleotide for upregulation of SCN1A expression
  modified_base 1: mod_base = OTHER; note = 2'OXethylxodification; phosphorothioated base
  modified_base 6: mod_base = OTHER; note = 2'OXethylxodification; phosphorothioated base
  modified_base 12: mod_base = OTHER; note = 2'OXethylxodification; phosphorothioated base
  modified_base 19: mod_base = OTHER; note = 2'OXethylxodification
  source 1..19: mol_type = other DNA
gtgactgtgcccattgctg

SEQ ID NO: 158 - DNA, 19 bp, synthetic construct
  misc_feature 1..19: note = synthetic oligonucleotide for upregulation of SCN1A expression
  modified_base 1: mod_base = OTHER; note = 2'OXethylxodification; phosphorothioated base
  modified_base 19: mod_base = OTHER; note = 2'OXethylxodification
  source 1..19: mol_type = other DNA
gtgactgtgcccattgctg

SEQ ID NO: 159 - DNA, 20 bp, synthetic construct
  misc_feature 1..20: note = synthetic oligonucleotide for upregulation of SCN1A expression
  modified_base 1: mod_base = OTHER; note = 2'OXethylxodification; phosphorothioated base
  modified_base 5: mod_base = OTHER; note = 2'OXethylxodification; phosphorothioated base
  modified_base 9: mod_base = OTHER; note = 2'OXethylxodification; phosphorothioated base
  modified_base 13: mod_base = OTHER; note = 2'OXethylxodification; phosphorothioated base
  modified_base 17: mod_base = OTHER; note = 2'OXethylxodification; phosphorothioated base
  modified_base 20: mod_base = OTHER; note = 2'OXethylxodification
  source 1..20: mol_type = other DNA
cctctttctggccttgcttc

SEQ ID NO: 160 - DNA, 26 bp, synthetic construct
  misc_feature 1..26: note = synthetic oligonucleotide for upregulation of SCN1A expression
  modified_base 1: mod_base = OTHER; note = 2'OXethylxodification; phosphorothioated base
  modified_base 6: mod_base = OTHER; note = 2'OXethylxodification; phosphorothioated base
  modified_base 11: mod_base = OTHER; note = 2'OXethylxodification; phosphorothioated base
  modified_base 17: mod_base = OTHER; note = 2'OXethylxodification; phosphorothioated base
  modified_base 21: mod_base = OTHER; note = 2'OXethylxodification; phosphorothioated base
  modified_base 26: mod_base = OTHER; note = 2'OXethylxodification
  source 1..26: mol_type = other DNA
gacaaccttgcagccactgatgatga

SEQ ID NO: 161 - DNA, 52 bp, synthetic construct
  misc_feature 1..52: note = synthetic oligonucleotide for upregulation of SCN1A expression
  misc_feature 51: note = deoxyribonucleotide
  source 1..52: mol_type = other DNA
atttaaacacggaagactttagtagtgctactaaagtcttccgtgtttaaat

SEQ ID NO: 162 - DNA, 52 bp, synthetic construct
  misc_feature 1..52: note = synthetic oligonucleotide for upregulation of SCN1A expression
  misc_feature 51: note = deoxyribonucleotide
  misc_feature 52: note = deoxyribonucleotide
  source 1..52: mol_type = other DNA
tcacaaattcagatcacccatcttctagaagatgggtgatctgaatttgtga

SEQ ID NO: 163 - DNA, 52 bp, synthetic construct; identical to SEQ ID NO: 161
  misc_feature 1..52: note = synthetic oligonucleotide for upregulation of SCN1A expression
  misc_feature 51: note = deoxyribonucleotide
  source 1..52: mol_type = other DNA